(19) United States
(12) Patent Application Publication
(10) Pub. No.: US 2009/0297534 A1
(43) Pub. Date: Dec. 3, 2009

(54) Catalytic Immunoglobulins BBK32 and Uses Therefor

(76) Inventors: Sudhir Paul, Missouri City, TX (US); Yasuhiro Nishiyama, Houston, TX (US); Carl V. Hanson, Berkley, CA (US); Marc Weksler, Tenafly, NJ (US)

Correspondence Address: Adler & Associates, 8011 Candle Lane, Houston, TX, 77071, US

(21) Appl. No.: 11/988,761

(22) PCT Filed: Jul. 13, 2006
(86) PCT No.: PCT/US06/27185

§ 371 Date: Jan. 27, 2009

Related U.S. Application Data
(60) Provisional application No. 60/698,742, filed on Jul. 13, 2005.

(51) Int. Cl. A61K 39/395

(52) U.S. Cl. 424/157.1; 424/130.1

(57) ABSTRACT

The present invention describes the composition of class and subclass selected pooled human immunoglobulins with catalytic activity, methods of preparation thereof, and therapeutic utility thereof.

### CROSS-REFERENCE TO RELATED APPLICATIONS

This U.S. national stage application is filed under 35 U.S.C. 363 and claims benefit of priority under 35 U.S.C. 365 of international application PCT/US2006/027185, filed Jul. 13, 2006, now abandoned, which claims benefit of priority under 35 U.S.C. 119(e) of provisional U.S. Ser. No. 60/698,742, filed Jul. 13, 2005, now abandoned.

### BACKGROUND OF THE INVENTION

1. Field of the Invention

The present invention relates generally to pooled human antibodies, and in particular, to class and subclass selected antibody preparations having catalytic activity.

2. Description of the Related Art

Without limiting the scope of the invention, its background is described in connection with catalytic antibodies. Generally, antibodies are composed of a light (L) chain and a heavy (H) chain. The variable regions of these chains are important in defining the paratope or antigen binding site conformation to one that binds antigen with high affinity. Some antibodies have the ability to catalyze chemical reactions through the binding of a substrate, its chemical conversion and release of one or more products. Catalytic antibodies have been described in several autoimmune diseases (1). Initially, it was assumed that the natural formation of catalytic antibodies by the immune system is a rare event representing the accidental generation of catalytic sites during the diversification of antibody V domains accompanying B lymphocyte maturation. However, advances in immunochemical technology have accelerated the identification of additional naturally occurring catalytic antibodies and elucidation of their mechanism of action. Polyclonal and monoclonal catalytic antibodies have been found with enzymatic mechanism similar to serine proteases, e.g., antibodies that hydrolyze the neuropeptide VIP (5). Other naturally occurring antibodies are known to hydrolyze DNA, phosphorylate proteins and hydrolyze esters (1). Immunoglobulins of the IgG and IgM class found in healthy individuals were described to possess a promiscuous proteolytic activity, evident from the cleavage of small tripeptide and tetrapeptide substrates (11). Antibodies with proteolytic and other catalytic activities have been characterized in the blood and mucosal secretions (12). The catalytic activity has been traced to nucleophilic sites of innate origin located in antibody germine variable regions (11).

Reduced cleavage of model peptide substrates by endogenous, naturally occurring catalytic antibodies has been correlated with the incidence of autoimmune disease (10) and with diminished survival of patients with septic shock (13). Thus, the catalytic function of the immunoglobulins can fulfill important protective roles in certain disease states. The kinetic characteristics of the endogenous, promiscuous catalytic antibodies suggest that they can effectively clear antigens that accumulate to large concentrations (11), e.g., certain autoantigens and bacterial antigens accumulating at the site of infection.

Antibodies that posses the catalytic activity of enzymes have the potential of generating potent therapeutic agents. Consequently, there has also been considerable interest in inducing the synthesis of catalytic antibodies on demand. For example, attempts have been made to induce catalytic antibodies by immunizing an animal with a stable analog of the ground state or transition state of the reaction to be catalyzed, and screening for antibodies that bind more strongly to the analog than to the corresponding substrate (14). Immunization with electrophilic antigens results in the synthesis of specific catalytic antibodies because of improvement in the natural nucleophilic reactivity of the antibodies combined with noncovalent recognition of epitope regions remote from the reaction center (15).

Antibodies, like enzymes, have a site that is chemically reactive with the substrate and expresses complementarity to the 3-dimensional and charge distribution structure of the transition state. Only a minority of the antibodies express the ability to catalyze the reaction of interest. Such antibodies can be identified by specific assays of catalytic transformation of individual polypeptides from among candidate antibody preparations. Certain other reactions, e.g., the ability to cleave small peptides in a manner that is comparatively independent of the precise structure of the peptides, are more frequently catalyzed by antibodies (10, 11).

Also critical is the maintenance of the antibodies in a catalytic conformation. Just as enzymes are denatured and lose their catalytic activity by treatment with buffers and compounds that perturb the 3-dimensional arrangement of their active sites, the catalytic activity of antibodies can readily be lost due to protein denaturation. This consideration is important in the utility of various catalytic antibody preparations. Very large doses of a poorly active catalytic antibody preparation must be used to achieve the desired biological effect compared to a highly catalytic antibody preparation. Another important issue is that individual antibodies display unique specificities for defined regions of the target antigen (epitopes), whereas different biological functions of the antigen are often mediated by distinct antigen regions. Thus, an individual catalytic antibody might neutralize a particular biological function of the antigen, but other functions can remain unaffected.

Combinations of monoclonal antibodies that bind a given antigen have been found to display antigen neutralizing activities superior to the individual monoclonal antibodies, suggesting that synergistic effects of combinations of antibodies are possible (16). Polyclonal antibody preparations from human sera are essentially mixtures of individual monoclonal antibodies. An increase in diversity of candidate therapeutic antibody preparations can be accomplished by pooling the polyclonal antibodies from many humans. Such pooled antibodies are commonly designated IVIG preparations (intravenously infused immunoglobulin preparations) and are marketed by several companies. Most IVIG preparations consist of pooled human antibodies of the IgG class. These IVIG preparations are obtained from the blood of humans by chemical procedures that assure purity (17) but do not take into account the requirement for maintenance of catalytic activity. Intravenous administration of IVIG preparations is well known to be therapeutic benefit in patients with immunodeficiency, infections and autoimmune disease, including bacterial sepsis, multiple sclerosis and idiopathic thrombocytopenic purpura. IVIG preparations have also been considered for the treatment of HIV infection, but their therapeutic benefit has not been established with certainty (18). In infectious disease, high affinity antibodies to antigens expressed by the infectious microorganism are a common finding. Intravenous infusion of pooled IgG from HIV infected subjects (HIVIG) has also been suggested as a treatment for HIV infection (19). Generally, the treatment entails administration of large quantities of IVIG preparations, for example, 1 gram/kilogram body weight.

The mechanism of IVIG therapeutic effects in different diseases has not been defined precisely, but several mechanisms have been proposed: (a) reversible neutralization of the bioactivity of antigens via steric hindrance due to antigen binding at antibody variable domains; (b) increased clearance of the antigen mediated by binding of antigen-antibody complexes to cells expressing Fc receptors; (c) binding of complement components at the Fc region of the antibody following complexation to antigens on the cell surface, resulting in antibody-dependent complement-mediated cellular lysis; and (d) activation of natural killer cells following antibody complexation to antigens on the cell surface, resulting in antibody-dependent cell-mediated lysis. The catalytic activity of IVIG preparations has not been described in the literature. Different classes of immunoglobulins, i.e., IgM, IgG, IgA, and IgE, mediate the effector functions of immunoglobulins with variable levels of efficiency. As noted previously, IVIG preparations are generally composed of IgG preparations. IgM class antibodies are described to catalyze the cleavage of certain substrates with superior efficiency than IgG class antibodies (11, 20).

The presence of antibodies that bind proteins specifically in IVIG preparations is of particular interest in regard to therapeutic utility. IVIG preparations can be expected to contain antibodies that bind microbial superantigens, defined as antigens bound by antibodies found in the preimmune repertoire without the requirement of adaptive maturation of antibody variable domains (21-23). Examples of superantigens are the HIV coat protein gp120, HIV Tat and Staphylococcal Protein A. In addition, the endogenous microbial flora found in healthy humans can stimulate the adaptive synthesis of antibodies that bind the microbial antigens and such antibodies may be present in IVIG preparations. The blood of humans also contains antibodies that bind a variety of autoantigens, including CD4 (24), amyloid β peptide (25) and VIP (26, 27), and the presence of IgG antibodies that bind amyloid β peptide in IVIG preparations has been reported (28).

The example of HIV gp120 as the target of pooled immunoglobulins such as conventional IVIG preparations is presented here to provide additional background for the present invention, and additional examples of other antigenic targets are noted throughout this filing. One of the key components in the host cell binding by HIV-1 is the gp120 envelope glycoprotein. Specifically, the binding of a conformational epitope of glycoproteins gp120 to CD4 receptors on host cells is the first step in HIV-1 infection. Additionally, gp120 exerts a toxic effect on cells that are not infected with HIV, including T cells and neurons (29-37). Therefore, the gp120 glycoprotein and its precursor gp160 glycoprotein are logical targets in the treatment of AIDS. It has been shown that monoclonal antibodies that bind the CD4 binding site of gp120 reduce viral infectivity (38, 39). The gp120 envelope glycoprotein expresses many other antigenic epitopes. Following infection with HIV, humans mount vigorous antibody responses to gp120, but most antibodies are directed to the hypervariable region of the protein, and are ineffectual in controlling infection. It is necessary for the antibody to recognize the conserved regions of gp120 to permit broad protection against diverse HIV strains, and broadly protective antibodies are to HIV are generally not produced following HIV infection. The superantigenic site of gp120 contains regions that are important in host cell CD4 binding, in particular the conserved region composed of residues 421-433 (40, 41). Catalytic antibodies to the superantigenic site of gp120 thus hold the potential of controlling infection, both by virtue of permanent degradation of gp120 and repeated use of a single antibody molecule for cleavage of many gp120 molecules.

The foregoing problems related to antigenic specificity and their catalytic activity have been recognized for many years. Numerous solutions have been proposed, but none of them adequately address all of the issues.

### SUMMARY OF THE INVENTION

The present invention is directed to an isolated and purified pooled immunoglobulin preparation comprising pooled immunoglobulins of defined class having catalytic activity. In a related invention the immunoglobulins are also defined by subclass.

The present invention also is directed to method for isolating and purifying pooled catalytic immunoglobulins described herein for therapeutic use. The method comprises the step of pooling the source fluids obtained from humans and fractionation of the immunoglobulins into a defined class and subclass fraction, where the fraction expresses catalytic activity. The present invention is directed to a related method providing a further step of adding a compound that binds and protects the catalytic site during the fractionation procedure, including but not limited to the substrate. The present invention is directed to another related method providing a further step of comparing the catalytic activity of antibody classes and subclasses against an antigen.

The present invention is directed further to a method for treating a patient. The method comprises providing an effective amount of pooled catalytic immunoglobulins of a defined class to a patient in need thereof.

Other and further aspects, features and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 demonstrates hydrolysis of EAR-AMC by CIVIGg, CIVIGm, CIVIGa and CIVIGas. The substrate EAR-AMC (0.2 mM) was incubated with CIVIG preparations (CIVIGg, 75 μg/mL; CIVIGm, 36 μg/mL; CIVIGa, 11 μg/mL; CIVIGas, 11 μg/mL; CIVIGg, CIVIGm and CIVIGa were prepared from a pool of blood from 35 humans (Gulfcoast Blood Bank) in 50 mM Tris.HCl, 0.1 M glycine, pH 8.0, containing 0.1 mM CHAPS at 37° C. The release of AMC was monitored periodically by fluorometry (λem 470 nm, λex 360 nm; Cary Eclipse spectrometer, Varian, Palo Alto, Calif.), normalized to 11 μg Ig/mL equivalent, and fitted to the equation: [AMC]=V·t, where V represents the specific activity (μM AMC/h/11 μg Ig/mL). CHAPS, 3-[(3-cholamidopropyl)dimethylammonio]-1-propanesulfonic acid.

FIG. 2 demonstrates hydrolysis of EAR-AMC by CIVIGg and the IgG fraction from IVIGs. Catalytic activity was measured as in FIG. 1 (IgG, 75 μg/mL).

FIG. 3 demonstrates hydrolysis of EAR-AMC by CIVIGm and the IgM fraction from IVIGs. Catalytic activity was measured as in FIG. 1. (IgM, 36 μg/mL).

FIG. 4 demonstrates hydrolysis of EAR-AMC by CIVIGa, CIVIGsa and IVIGa. Catalytic activity was measured as in FIG. 1 (CIVIGa and CIVIGas, 11 μg/mL; Pentaglobin IgA, 80 μg/mL). The activity was normalized to 80 μg Ig/mL equivalent.

FIG. 5 demonstrates cleavage of gp120 by IgG, IgM and IgA from human blood and saliva. Bt-gp120 (1.6 Bt/protein) was incubated with human serum IgG, IgM, and IgA and saliva IgA prepared from 4 individual sets of specimens. Shown are example streptavidin-peroxidase stained blots of reducing SDS-gels showing cleavage of Bt-gp120 by immunoglobulins purified from serum and saliva from one donor. Bt-gp120, 0.1 μM; IgG, 135 μg/mL; IgM, 180 μg/mL; IgA, 144 μg/mL; 37° C., 17 h.

FIG. 6 demonstrates preferential cleavage of gp120 by CIVIGa and CIVIGsa. Biotinylated proteins studied are gp120, extracellular domain of epidermal growth factor receptor (exEGFR), bovine serum albumin (BSA), C2 domain of human coagulation factor VII (C2), and HIV-Tat. Shown are streptavidin-peroxidase stained blots of reducing SDS-gels showing biotinylated proteins (0.1 μM) incubated with CIVIGa, CIVIGas (160 μg/mL) or diluent for 17 h in 50 mM Tris.HCl, 0.1 M glycine, pH 8.0, containing 1 mM CHAPS and 67 μg/mL gelatin.

FIG. 7 demonstrates cleavage of protein A and sCD4 by CIVGa and CIVIGas. Shown are streptavidin-peroxidase stained blots of reducing SDS-gels showing sCD4 and protein A (0.1 μM; bioninylated) incubated with CIVIGa, CIVIGas (160 μg/mL), or diluent for 17 h in 50 mM Tris HCl, 0.1 M glycine, pH 8.0, containing 1 mM CHAPS and 67 μg/mL gelatin. Protein A was iodinated prior to biotinylation to inactivate the Fc binding site while leaving intact the recognition of this protein as a superantigen by the V domains.

FIG. 8 demonstrates that HIV-Tat cleavage by CIVIGm is evident by depletion 14-kD band and lack of cleavage by CIVIGa, CIVIGas, and CIVIGg. Shown are streptavidin-peroxidase stained blots of reducing SDS-gels showing HIV-Tat (0.1 μM; biotinylated) incubated with diluent (lane 1), CIVIGa (160 μg/mL, lane 2), CIVIGas (160 μg/mL, lane 3), CIVIGg (160 μg/mL, lane 4) and CIVIGm (180 μg/mL, lane 5; 810 μg/mL, lane 6) for 17 h in 50 mM Tris HCl, 0.1 M glycine, pH 8.0, containing 1 mM CHAPS and 67 μg/mL gelatin.

FIGS. 9A-9B demonstrate superior HIV-1 neutralization activity of CIVIG preparations to commercial IVIG. FIG. 9A shows HIV neutralization by CIVIG preparations. HIV-1 (ZA009; R5, clade C) was incubated with CIVIG preparations and commercial IVIGs at varying concentrations (2.5-250 μg/mL), then allowed to infect PBMC. HIV-1 neutralization activity is expressed as % decrease of p24 concentrations as compared to those treated with diluent (phosphate-buffered saline; PBS). FIG. 9B shows low to negligible HIV neutralization by commercial IVIGs. Neutralization activity was measured as in FIG. 9A.

FIGS. 10A-10B demonstrate inhibition of CIVIG-mediated HIV neutralization by gp120 peptide-CRA. CIVIGm (FIG. 10A) and CIVGa (FIG. 10B) were incubated for 30 min with gp120 peptide-CRA (100 μM) or diluent, and the residual neutralization activity was determined as in FIG. 9 (CIVIGm, 10 μg/mL; CIVGa, 2 μg/mL).

FIGS. 11A-11B demonstrate hydrolysis of Glu-Ala-Arg-AMC by IgA purified from human sera and saliva. FIG. 11A depicts scatter plots showing Glu-Ala-Arg-AMC hydrolyzing activity of purified IgA obtained from the serum and saliva of 4 humans. Connected symbols signify serum and salivary IgA from the same individuals. The substrate (0.2 mM) was incubated in the presence of IgA (32 μg/mL) in triplicates and the fluorescence increase was monitored over 20 h. Each data point is the mean velocity (Δ FU)/h) obtained from least-square-fits to FU=V·t (r², ≧0.99). Background hydrolysis of the substrate incubated in the absence of IgA was negligible (<0.1 FU/h). FIG. 11B depicts progress curves of Glu-Ala-Arg-AMC hydrolysis by IgA, IgG and IgM from a pool of sera of 34 healthy individuals. The substrate (0.4 mM) was incubated in the presence of IgA (11 μg/mL), IgG (75 μg/mL), or IgM (36 μg/mL). EAR-AMC hydrolyzed was determined by measuring AMC fluorimetrically. Shown are values expressed per μg Ab in the 50 μL reaction (mean±SD; n=3) and least-square-fit curves to [AMC]=V·t (r², ≧0.98).

FIGS. 12A-12B demonstrate IgA purity. FIG. 12A depicts reducing SDS-gel lanes showing electrophoretic homogeneity of IgA samples from human sera (pool of 34) and saliva (pool of 4). Lanes 1-3 are serum IgA subunits stained with Coomassie Blue, anti-α chain, and anti-κ/λ chain, respectively. Lanes 4-7 are salivary IgA subunits stained with Coomassie Blue, anti-α chain, and anti-κ/λ chain, and anti-secretory component, respectively. FIG. 12B depicts progress curves showing comparable Glu-Ala-Arg-AMC hydrolyzing activity of a serum IgA sample before and after denaturing gel filtration conducted in 6 M guanidine hydrochloride. The 170-kDa IgA fraction was assessed for catalytic activity as in FIG. 11. IgA, 8 μg/mL; Glu-Ala-Arg-AMC (0.4 mM).

FIG. 13 demonstrate comparative amidolytic activity of pooled IgA and commercially available IVIG preparations. Reaction conditions as in FIG. 11B.

FIG. 14 demonstrate apparent kinetic parameters for IgA catalyzed Glu-Ala-Arg-AMC hydrolysis. Shown are data for two serum IgA preparations (subject ID 2288 and 2291). Initial velocities (V) are fitted to the Michaelis-Menten equation V=k_{cat}·[Ab]·[S]/(Km+[S]) by non-linear regression (r² 0.998). [Ab], antibody concentration; [S], initial substrate concentration.

FIGS. 15A-15C demonstrate the reaction of IgA with serine protease inhibitors. FIG. 15A depicts structures of active site serine protease probes. Phosphonates 1a and 1b phosphonylate the active site nucleophiles of trypsin-like serine proteases and Abs and inhibit their proteolytic activity. Compound 2 is a 1 a-derivative devoid of the positively charged amidino mimetic of Arg/Lys. The amidino group is required for phosphonate reactivity with proteolytic IgG and IgM Abs. FIG. 15B depicts inhibition of IgA-catalyzed Glu-Ala-Arg-AMC hydrolysis by serine protease inhibitors. The substrate (0.4 mM) was incubated with serum IgA (8 μg/mL) in the presence and absence of 1a or DFP (10, 30, 100, 300 μM) and the AMC fluorescence monitored over 23 h. Values of AMC release at various inhibitor concentrations were fitted to the equation [AMC]/[AMC]max=1−e^{-k·t}, where [AMC]ₘₐₓ and k represent, respectively, the extrapolated maximum value of AMC release and the first-order rate constant (r², >0.98). The progress curves in the presence of inhibitor were hyperbolic as predicted from the irreversible character of the inhibition with IgA. The residual activities in the presence of inhibitor (Vᵢ) were computed as the tangents of the progress curves at 23 h. Percent inhibition was computed as 100(V−Vᵢ)/V, where V represents the velocity in the absence of inhibitor. Data are means±SD of three replicates. IC₅₀ values were extracted from least-square-fits to the equation, % inhibition=100/(1−10^{log EC50-log [1a]}) (r² >0.92). FIG. 15C depicts reducing SDS-gel lanes showing 1a-adducts of IgA subunits. Shown are streptavidin-peroxidase stained blots of the following reaction mixtures (6 h). Lane 1, serum IgA and 1a; lane 2, serum IgA and 2; lane 3, saliva IgA and 1a; saliva IgA and 2. H and L denote, respectively, 1a adducts of heavy chain and light chain. IgA, 160 μg/mL; 1a and 2 (0.1 mM).

FIG. 16 demonstrates stoichiometry of monoclonal IgA reaction with phosphonate 1b. Monoclonal IgA (ID 2582; 1.6 mg/mL) was incubated with 1b (2.5-20 μM). After 18 h, the residual activity was measured by incubating 1b-treated IgA (24 μg/mL) with Glu-Ala-Arg-AMC (0.4 mM). Shown is the plot of residual catalytic activity vs [1b]/[IgA]. The x-intercept shown in the plot was determined from the least-square fit for data points at [1b]/[IgA] ratio≦1 (r² 0.93).

FIGS. 17A-17C demonstrate cleavage of Bt-gp120 by serum and salivary IgA from HIV-seronegative humans. FIG. 17A depicts streptavidin-peroxidase stained blots of reducing SDS-gels showing time-dependent cleavage of Bt-gp120 (0.1 μM) by pooled polyclonal serum IgA (160 μg/ml) and salivary IgA (32 μg/ml) from 4 humans. Diluent lane, gp120 incubated with diluent instead of IgA. OE, overexposed lane showing Bt-gp120 incubated for 46 h with salivary IgA. Product bands at 55, 39, 32, 25 and 17 kD are visible. FIG. 17B depicts scatter plot of gp120 cleaving activity of salivary IgA, serum IgA and serum IgG from 4 humans. Ab concentrations: salivary IgA, 32 μg/ml; serum IgA, serum IgG and commercial IVIG preparations (Intratect, Gammagard, Inveegam), 144 μg/ml. Reaction conditions: 17 h, 37° C., 0.1 μM Bt-gp120. Shown are activities expressed per unit mass Ab. Solid lines are means (salivary IgA and serum IgA, respectively, 6053±1099 and 391±183 nM/h/mg Ab; cleavage by IgG and IVIG preparations was below the detection limit). FIG. 17C depict typical reducing SDS-electrophoresis (4-20% gels) results showing human serum IgA and salivary IgA purified by affinity chromatography on immobilized anti-IgA Ab and stained with Coomassie blue (lanes 1 and 4, respectively), anti-α chain Ab (lanes 2 and 5, respectively), and κ/λ Ab (lane 3 and 6, respectively). Lane 7 shows salivary IgA stained with anti-secretory component Ab.

FIGS. 18A-18C demonstrate gp120 cleavage by refolded polyclonal IgA following denaturing gel filtration and by monoclonal IgAs from patients with multiple myeloma. FIG. 18A depicts gel filtration chromatograms of pooled human salivary IgA (solid line) and serum IgA (dashed line) conducted in 6 M guanidine hydrochloride. Salivary IgA (0.8 mg) and serum IgA (1.6 mg) purified by anti-IgA affinity chromatography were applied to the column. Fractions from salivary IgA corresponding to 433-915 kD (solid bar a) or serum IgA corresponding to 153 kD (solid bar b) were pooled and analyzed further. Silver-stained SDS-electrophoresis gels of salivary IgA fraction a and serum IgA fraction b are shown. sc, H and L denote, respectively, the secretory component, heavy chain and light chain bands. FIG. 18B depicts streptavidin peroxidase-stained electrophoresis blots showing Bt-gp120 cleavage by salivary and serum IgA following denaturing gel filtration. Fractions a and b were dialyzed against Tris-Gly buffer, pH 7.7, prior to the assay. Shown are Bt-gp120 (0.1 μM) incubated with diluent (lane 1), salivary IgA (32 μg/ml, lane 2) and serum IgA (32 μg/ml, lane 3) for 45 h. FIG. 18C depicts a scatter plot of Bt-gp120 cleaving activities of monoclonal IgAs. Bt-gp120, 0.1 μM; IgA, 75 μg/ml; Reaction time, 21 h. Dashed line corresponds to background value (incubations with diluent instead of IgA)+3 standard deviations.

FIGS. 19A-19C depict the structure of EP-hapten 1 and its inhibition of catalysis and irreversible binding. FIG. 19A shows that the non-electrophilic phosphonic acid hapten 2 is structurally identical to hapten 1 except for the absent phenyl groups. In FIG. 19B gp120 (0.1 μM) was incubated with salivary IgA (2 μg/ml) or serum IgA (160 μg/ml) in the absence or presence of EP-hapten 1 and control hapten 2 (1 mM) for 8 h before incubation with non-biotinylated gp120 for 16 h. The residual intact gp120 was measured by densitometry following SDS-electrophoresis and by staining of the blots with peroxidase-conjugated polyclonal anti-gp120. % Inhibition=100−[(gp120 cleaved in the presence of inhibitor)/(gp120 cleaved in the absence of inhibitor)×100]. Values are means of duplicates. FIG. 19C shows streptavidin-peroxidase stained blots of reducing SDS-gels showing EP-hapten 1-treated salivary IgA (lane 1) and serum IgA (lane 3). Also shown are hapten 2-treated salivary IgA (lane 2) and serum IgA (lane 4). H and L denote heavy and light chain subunit bands, respectively.

FIGS. 20A-20C demonstrates inhibition of IgA catalyzed gp120 cleavage by Glu-Ala-Arg-AMC and active site titration of the IgA. FIG. 20A shows the plot of residual catalytic activity vs [EP-hapten 3]/[IgA] (least-square fit, r² 0.84). The x-intercept of the residual activity (%) versus [EP-hapten 3]/[IgA] plot was 2.4. FIG. 20B depicts streptavidin-peroxidase stained SDS-gel blots (reducing conditions) showing Bt-gp120 (0.1 μM) incubated in diluent (lane 1) and monoclonal IgA (80 μg/ml, from multiple myeloma subject 2582) in the absence (lane 2) or presence of Glu-Ala-Arg-AMC (lane 3; 0.2 mM). For active site titration, the monoclonal IgA (10 μM, assumed mass 170 kD) was preincubated for 18 h in the absence or presence of EP-hapten 3 (2.5-20 μM), Glu-Ala-Arg-AMC was added to a concentration of 0.4 mM and catalytic activity was measured by fluorimetry. FIG. 20C is EP-hapten 3 which is the non-biotinylated version of EP-hapten 1.

FIG. 21 demonstrates preferential cleavage of gp120 by IgA and sIgA. Biotinylated (Bt) proteins studied are gp120, soluble epidermal growth factor receptor (sEGFR), bovine serum albumin (BSA), C2 domain of human coagulation factor VIII (C2), and HIV Tat. Shown are streptavidin-peroxidase stained blots of reducing SDS-gels of the proteins (0.1 μM) incubated (17 h) with serum IgA, salivary IgA (both 160 μg/ml) or diluent.

FIGS. 22A-22D demonstrate IgA interactions with EP-421-433. FIG. 22A depicts structures of EP-421-433 (SEQ ID NO: 18) and the control electrophilic peptide (EP-VIP; SEQ ID NO: 19). R1, amidinophosphonate mimetic of gp120 residues 432-433 linked to Gly431 carboxyl group; R2, amidinophosphonate group linked to Lys side chain amine. FIG. 22B shows inhibition of IgA catalyzed gp120 cleavage by EP-421-433. Salivary IgA (16 μg/ml) or serum IgA (160 μg/ml) were preincubated (6 h) with EP-421-433 or EP-VIP (100 μM), the reaction mixtures were incubated further for 16 h following addition of gp120 (0.1 μM). Inhibition of gp120 cleavage determined as in FIG. 18B. FIG. 22C shows irreversible binding of EP-421-433 by salivary IgA and serum IgA. Shown are streptavidin-peroxidase stained blots of reducing electrophoresis gels of salivary IgA (80 μg/ml) incubated with EP-421-433 (lane 1), EP-VIP (lane 2) or EP-hapten 1 (lane 3); and serum IgA (80 μg/ml) incubated with EP-421-433 (lane 4), EP-VIP (lane 5) or EP-hapten 1 (lane 6). EP-probe concentration, 10 μM; reaction time, 21 h. H and L denote heavy chain and light chain bands. FIG. 22D shows inhibition of irreversible IgA:EP-421-433 binding by gp120 peptide 421-435. Salivary IgA (80 μg/ml) was treated with gp120 peptide 421-435 (100 μM) or diluent followed by addition of EP421-433 (10 μM) and further incubation for 21 h. EP-421-433 adducts were detected as in panel C and the band intensities determined by densitometry. Plotted values represent the sum of the heavy and light chain subunits.

FIG. 23 identifies peptide bonds cleaved by salivary IgA. Shown is a typical Coomassie blue-stained SDS-gel electrophoresis lane of gp120 (270 μg/ml) digested with IgA (80 μg/ml; 46 h). N-terminal sequences of the resultant polypeptide fragments are reported using single letter amino acid code. Values in parentheses represent quantities (pmol) of amino acids recovered in the individual sequencing cycles. Prior to electrophoresis of the gp120 digest, IgA was removed by chromatography on immobilized anti-α column.

FIGS. 24A-24C demonstrate HIV neutralization by Abs from HIV-seronegative humans. FIG. 24A depicts the neutralizing potency of IgA and IgG Abs purified from pooled serum or saliva of 4 human subjects. HIV-1 strain, 97ZA009; host cells, phytohemagglutinin-stimulated PBMCs. Abs were incubated with the virus for 24 h. Values are expressed as percent reduction of p24 concentrations in test cultures compared to cultures that received diluent instead of the Abs (means±s.d. of 4 replicates). FIG. 24B shows inhibition of IgA neutralizing activity by EP-421-433. IgA purified from human serum (2 μg/ml) was preincubated (0.5 h) with EP-421-433 (100 μM), control EP-VIP or diluent, and the residual HIV neutralizing activity measured as in panel A. Data are expressed relative p24 levels observed in the absence of antibody. FIG. 24C shows time-dependent HIV neutralizing activity. HIV was preincubated with the salivary or serum IgA for 1 h and the neutralizing activity measured as in FIG. 24A.

FIGS. 25A-25B demonstrate increased gp120-cleaving IgAs in HIV infected men with slow progression to AIDS. FIG. 25A shows gp120 cleaving activities of IgA fractions. FIG. 25B shows blood CD4+ T cell counts. Bt-gp120 cleavage was determined by SDS-electrophoresis and the activity expressed as the intensity of the 55-kD fragment (AVU). Bt-gp120, 0.1 μM; IgA, 80 μg/ml; reaction time, 48 h. Each point represents one study subject. Values are means of duplicates. SP, slow progressors; RP, rapid progressors. Bleed 1, RP bleed 2 and SP bleed 2 samples were collected, respectively, 6 months, 1-5 years and 5.5 years after seroconversion. * P=0.035 vs HIV seronegative group; ** P<0.0001 vs RP group or HIV seronegative group.

FIGS. 26A-26B demonstrate heterologous HIV strain neutralization by serum IgA from presumptive clade B infected LTNPs. FIG. 26A shows neutralization of clade C strain ZA009 (R5 coreceptor) by IgA from 3 long-term nonprogressors (>18 years). IgA incubated 1 h with HIV before assaying infectivity using human PBMCs. Means of 4 replicates. FIG. 26B shows the consensus V3 region 306-325 and 421-433 region sequences compared to corresponding strain ZA009 sequences. Dot, identity; dash, gap.

FIG. 27 demonstrates cleavage of Aβ1-40 by human IgM and IgG. Aβ1-40 (100 μM) incubated for 3 days at 37° C. with IgG (1.6 μM) or IgM (34 nM) pooled from 6 non-AD subjects each of age <35 years (young) or >72 years (old). Reactions analyzed by reversed phase HPLC (gradient of 10% to 80% acetronitrile in TFA, 45 min; detection: A220). The product peptide profile for all antibodies studied was similar to that shown in FIGS. 28A-28B and indicated a major cleavage at Lys28-Gly29 and a minor cleavage at Lys16-Leu17. Rates computed from the area of the Aβ1-28 peak interpolated from a standard curve constructed using increasing amounts of synthetic Aβ1-28. *P<0.0044; **P<0.035. Two-tailed unpaired t-test.

FIGS. 28A-28C illustrate the polymorphic character of Aβ1-40 cleaving IgG and IgM antibodies from different human subjects. All human subjects in FIGS. 28A-28B were >72 years old. FIG. 28C shows Aβ1-40 cleavage by monoclonal IgMs purified from patients with Waldenström's macroglobulinemia. Two monoclonal IgMs with catalytic activity were identified. One of these, IgM Yvo displayed near-equivalent catalytic activity following purification by 4 cycles of cryoprecipitation (▪) and further affinity chromatography on immobilized anti-IgM antibody (x), suggesting purification to constant specific activity. Aβ1-40 (100 μM) incubated for 3 days at 37° C. with IgG (1.5 μM) or IgM (27 nM). Cleavage rates determined as in FIG. 26.

FIGS. 29A-29C identify peptide bonds in Aβ1-40 (SEQ ID NO: 1) cleaved by monoclonal IgM Yvo. FIG. 29A shows reversed phase HPLC profiles of Aβ1-40 (100 μM) incubated with monoclonal IgM (Yvo, 600 nM; 24 h; gradient of 10% to 80% acetronitrile in TFA, 45 min). Detection at 220 nm. Top and bottom HPLC traces are the control IgM Yvo alone and the control Aβ1-40 alone. FIG. 29B identifies the peak at retention time 21.2 min as the Aβ1-40 fragment by electrospray ionization-mass spectroscopy (ESI-mass spectroscopy). FIG. 29C shows a zoom scan of spectrum region around m/z peak 1085.5, corresponding to the exact theoretical m/z for singly charged (M+H)⁺ ion of Gly-Ala-Ile-Ile-Gly-Leu-Met-Val-Gly-Gly-Val-Val (SEQ ID NO: 2) (Aβ1-40). The 1 mass unit peak-splitting evident in the zoom scan reflects the natural isotopic distribution of singly charged Aβ29-40 ions. Further MS/MS analysis of the singly charged peptide confirmed its designation as Aβ29-40 based on detection of the expected b- and y-fragment ion series (not shown).

FIGS. 30A-30D identify peptide bonds in Aβ1-40 cleaved by polyclonal IgM (pooled from 6 aged subjects). FIG. 30A shows reversed phase HPLC profiles of Aβ1-40 (100 μM) incubated with IgM (400 nM; 74 h; gradient of 10% to 80% acetronitrile in TFA, 45 min). Detection at 220 nm. Top and bottom HPLC traces are the control IgM alone and the control Aβ1-40 peptide alone. FIG. 30B identifies the peak at retention time 10.2 min as the Aβ1-16 fragment by ESI-mass spectroscopy. FIGS. 30C-30D show zoom scans of spectrum region around m/z peaks 652.6 and 978.0, respectively, that correspond to the exact theoretical m/z for triply and doubly charged (M+3H)³⁺ and (M+2H)²⁺ ion of Asp-Ala-Glu-Phe-Arg-His-Asp-Ser-Gly-Tyr-Glu-Val-His-His-Gln-Lys (Aβ1-16) (SEQ ID NO: 3). The 0.3 or 0.5 mass unit peak-splitting evident in the zoom scan reflects the natural isotopic distribution of triply and doubly charged Aβ29-40 ions. Further MS/MS analysis of the triply charged peptide confirmed its designation as Aβ1-16 based on detection of the expected b- and y-fragment ion series (not shown).

FIGS. 31A-31B depict the morphology of Aβ1-40 assemblies in the presence of catalytic IgM Yvo or noncatalytic IgM 1816. FIG. 31A shows atomic force micrographs of Aβ1-40 (100 μM) maintained at 37° C. in PBS containing the monoclonal IgM (0.5 μM) for 6 days. x, y, z range: 10 μm, 10 μm, 10 nm. Arrows labeled as PF, SF, and O denote, respectively, peptide protofibrils, peptide short fibrils, and oligomers. Controls included freshly prepared reaction mixtures of the peptide and catalytic IgM (day 0) as well as the peptide incubated with noncatalytic IgM. Note greatly reduced peptide aggregates in the presence of IgM Yvo at day 6. FIG. 31B shows decreased Aβ1-40 assemblies in the presence of catalytic IgM Yvo on day 12 compared to day 6. Reaction conditions and AFM as in FIG. 30A. Arrow meanings as in FIG. 31A. MF, peptide mature fibrils.

FIGS. 32A-32C characterize the IgM Yvo mechanism of catalysis. FIG. 32A shows streptavidin-peroxidase stained reducing SDS-electrophoresis gel lanes showing irreversible binding of the biotinylated serine protease inhibitor, Bt-Z-2Ph, diphenyl-N-[6-(biotnamido)hexanoyl]-amino(4-amidinophenyl)-methane phosphonate (500 μM) by IgM Yvo (0.1 μM; Lane 1) and lack of reactivity of the IgM with the control probe devoid of covalent reactivity, Bt-Z-20H, dihydroxy-N-[6-(biotnamido)hexanoyl]-amino(4-amidinophenyl)-methane phosphonate under identical conditions (Lane 2). The electrophilicity of the phosphorus atom in the control probe is poor, resulting in its failure to react with enzymatic nucleophiles. FIG. 32B shows stoichiometric inhibition of IgM Yvo-catalyzed Boc-Glu(OBzl)-Ala-Arg-AMC, N-tert-butoxycarbonyl-γ-benzyl-Glu-Ala-Arg-4-methylcoumaryl-7-amide hydrocloride, hydrolysis by the serine protease inhibitor Cbz-Z, diphenyl N-(benzyloxycarbonyl)-amino(4-amidinophenyl)methanephosphonate. Shown is the plot of residual catalytic activity of the IgM measured as the fluorescence of the aminomethylcoumarin (AMC) leaving group in the presence of varying Cbz-Z concentrations (0.05, 0.15, 0.5, 1, and 2 μM). Residual activity determined as 100 Vi/V, where V is the velocity in the absence of inhibitor and Vi is a computed value of the velocity under conditions of complete inhibitor consumption. Vi values were obtained from least-square fits to the equation [AMC]=Vi·t+A(1−e^{kobs·t}), where A and k_{obs} represent, respectively, the computed AMC release in the stage when inhibitor consumption is ongoing and the observed first-order rate constant, respectively (r² for individual progress curves, >0.96). The equation is valid for reactions with an initial first-order phase and a subsequent zero-order phase. The value of the x-intercept (0.94) was determined from the least-square fit for data points at [Cbz-Z]/[IgM active sites] ratios <2 (1 mole IgM=10 moles IgM active sites). The data suggest that the catalytic activity is attributable in its entirety to the IgM active sites. FIG. 32C shows progress curves for cleavage of Boc-Glu(OBzl)-Ala-Arg-AMC (200 μM) by IgM Yvo (10 nM) in the absence and presence of Aβ1-40 (30 and 100 μM). The observed inhibition suggests that Boc-Glu(OBzl)-Ala-Arg-AMC and Aβ1-40 are cleaved by the same active sites of IgM.

FIGS. 33A-33B demonstrate increased Aβ hydrolysis by IgMs from patients with Alzheimer's disease. FIG. 33A shows values of ¹²⁵I−Aβ40 (0.1 nM) hydrolysis (means of duplicates) incubated for 68 h with the IgM preparations (0.023 mg/ml) purified from AD patients (n=23) and elderly, non-demented control subjects (n=25). Each point represents a different human subject. FIG. 33B shows streptavidin-peroxidase stained blots of reducing SDS-gels showing reaction mixtures of biotinylated soluble epidermal growth factor receptor, BSA and ovalbumin (0.1 μM) incubated for 65 h with diluent or pooled IgM (0.22 mg/ml) from AD patients 2037, 2039, 2041, 2043, and 2044.

FIG. 34 demonstrates adaptive catalyst selection. Most Ab responses tend to disfavor improved catalytic turnover, because antigen digestion and release from the B cell receptor (BCR) will induce cessation of cell proliferation. However, there is no hurdle to increased BCR catalytic rates up to the rate of transmembrane BCR signaling. Under certain conditions, further improvements in the rate are feasible, e.g., increased transmembrane signaling rate that may be associated with differing classes of BCRs (μ, α class) or CD19 overexpression, or upon stimulation of the B cells by an endogenous or exogenous electrophilic antigen.

FIG. 35 illustrates that trimeric gp120 found on the surface of the HIV virus is essential for the entry into host cells via binding to CD4 and chemokine receptors. Polyclonal and monoclonal Abs that hydrolyze gp120 by recognizing the superantigenic site of the protein have been identified in uninfected individuals. These Abs appear to constitute an innate defense system capable of imparting resistance or slowing the progression of HIV infection.

### DETAILED DESCRIPTION OF THE INVENTION

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “or combinations thereof” refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the terms “abzyme” or “catalytic immunoglobulin” are used interchangeably to describe at least one or more antibodies possessing enzymatic activity.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims. While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

This invention addresses the need for improved compositions and methods for the preparation of antibodies that display high level catalytic activity and the desired bioactivity profiles. The improved compositions consist of pooled IgA, IgM and IgG antibodies that are promiscuous with respect to their antigenic specificity or are targeted to individual antigens. The antibodies may or may not include accessory molecules, e.g., the J-chain or the secretory component. A preferred embodiment of the invention is the use of pooled mucosal antibodies as catalytic immunoglobulin preparations. Disclosed are unexpected findings indicating that the mucosal milieu favors the synthesis of IgA class antibodies displaying high level catalytic activity. Such antibodies are found, for example in human saliva. Pooling of the catalytic immunoglobulins from different humans is employed in the present invention as a strategy to increase the diversity of antigenic specificities targeted by the immunoglobulin mixtures. Also disclosed is the unexpected finding that polyclonal catalytic immunoglobulins display catalytic activity greater than observed in a panel of monoclonal antibodies, indicating that mixtures of antibodies can display catalytic activity superior to homogeneous antibody preparations.

More particularly, the present invention includes an isolated and purified pooled immunoglobulin preparation of a defined class having catalytic activity. The immunoglobulins may also defined by subclass. In one embodiment, the pooled immunoglobulins are isolated from four, ten, twenty, thirty, thirty-five, fifty, one-hundred or more humans. The immunoglobulins may be isolated from mucosal secretions, saliva, milk, blood, plasma or serum. The defined class may be immunoglobulins IgA, IgM, IgG or mixtures and a combination thereof. Examples of catalytic reactions that may be catalyzed by the immunoglobulins may include, e.g., amide bond cleavage, peptide bond cleavage. The immunoglobulin class and/or subclass is selected based on a comparison of catalytic activity of various immunoglobulin classes and subclasses against a specific target antigen. The target of the catalytic reaction entails cleavage of a peptide bond HIV gp120, HIV Tat, Staphylococcal Protein A, CD4 or in amyloid beta peptide. In one specific example, the immunoglobulin class is selected based on a comparison of catalytic cleavage of amide bonds in peptide-aminomethyl coumarin antigens.

When prepared as a formulation of catalytic immunoglobulins of a defined class, these may be used in the prevention or therapy of HIV-1 infection by intravenous, intravaginal or intrarectal administration. Alternatively, the catalytic immunoglobulin formulation may be used to treat a bacterial infection, septic shock, autoimmune disease, Alzheimer's disease or a combination thereof by intravenous administration. The isolated and purified pooled catalytic immunoglobulins may be adapted for therapeutic use and isolated by pooling the source fluids obtained from humans and fractionation of the immunoglobulins into a defined class and subclass fraction, wherein the fraction expresses catalytic activity. The catalytic immunoglobulins may be isolated and purified from one or more classes and subclasses against an antigen by, e.g., fractionation and/or chromatography using antibodies to human IgA, IgM or IgG; or immunoglobulin binding reagents, Protein G, Protein A, Protein L; or electrophilic compounds capable of binding the nucleophilic site of the immunoglobulins; or mixtures and combinations thereof. Other examples of fractionation procedures for use in the methods of the present invention include, e.g., ion exchange chromatography, gel filtration, chromatography on lectins, chomatofocusing, electrophoresis or isoelectric focusing.

Several methods useful in the preparation and characterization of pooled immunoglobulins with high level catalytic activities are disclosed, for example, an immunoglobulin preparation that is selected for a defined class (IgM, IgG, IgA) of immunoglobulins. Pooled abzymes belonging to a defined immunoglobulin subclass (e.g., IgA1, IgA2) can also be obtained by suitable biochemical fractionation methods available in the art. The source of the pooled abzymes can be mucosal secretions such as saliva or blood pooled from human subjects, and any combination thereof.

In one embodiment of the invention, the pooled abzymes are prepared by affinity chromatography using immobilized antibodies to human IgA and/or IgM and/or IgG, instead of harsh chemical treatments that result in loss of catalytic activity. Immunoglobulin binding reagents like Protein G, Protein A or Protein L may also be used for this purpose. Also disclosed is the use of affinity chromatography procedures involving immobilized electrophilic compounds that are capable of selectively binding the nucleophilic site of the abzymes. In addition, common separations known to persons of ordinary skill in the art may be used, including but not limited to ion exchange chromatography, gel filtration, chromatography on lectins, chromatography on immunoglobulin binding proteins, chromatofocusing, electrophoresis or isoelectric focusing.

An important aspect of the invention is the analysis of the candidate pooled abzyme preparations at various stages of fractionation for the expression of catalytic activity. Depending on the intended use of the pooled abzyme preparation, the substrate or target may be, for example, small peptides, gp120 or amyloid β peptide. In another example, the present invention also provides a method of preparation of pooled abzymes by fractionation into a substrate specific fraction, wherein the latter fraction has catalytic activity. The method can further include comparing the catalytic activity of the abzymes against a specific target or model substrates that serve as indicators of promiscuous catalytic activity.

In another example, the present invention discloses abzymes that have proteolytic activity resulting in the cleavage of HIV gp120, amyloid β peptide, HIV Tat, Protein A or CD4.

In accordance with the present invention, a method of treatment of a patient, the pooled abzyme preparation may be used to treat a variety of diseases, e.g., autoimmune diseases, Alzheimer's disease, bacterial infection, septic shock, viral infections, multiple sclerosis and idiopathic thrombocytopenia purpura. The method provides for administering a pooled, class selected, substrate specific or promiscuous abzyme preparation to the patient. Depending on its intended use, the pooled abzyme preparation may be administered via numerous routes, including, but not limited to intravenous, intraperitoneal, intravaginal or intrarectal administration.

### Pooled Immunoglobulins for Therapy

Intravenous infusion of immunoglobulins of the IgG class prepared from the pooled serum of humans (commonly designated IVIG preparations) is currently employed for treatment of several diseases. The majority of marketed IVIG preparations are composed of purified IgG antibodies; however, a more complete IVIG preparation composed of IgG, IgM and IgA formulated in approximately the same proportion as found in human serum is also available, Pentaglobin. IVIG is generally prepared without regard to retention of the catalytic activity of antibodies, and comparatively harsh chemical methods are employed in the preparation procedures (42). Certain newer IVIG preparations incorporate chromatographic methods to improve purity. To minimize transmission of viral infections, filtration and/or viral inactivation procedures are also incorporated in IVIG preparation.

Circulating antibodies in the blood of healthy adult humans have been described to bind a variety of autoantigens and foreign antigens, e.g., amyloid β peptides, CD4, VIP, gp120 and Tat. Some of these antibodies have also been described in conventional IVIG preparations, e.g., antibodies that bind amyloid β peptides. Recently, conventional IVIG administered to patients with Alzheimer's disease has been suggested to improve cognitive performance. In autoimmune disease, high affinity antibodies to autoantigens are produced by the immune system, including antigens that are targets of certain therapeutic interventions, for example, antibodies to CD4 antigen for therapy of certain lymphomas.

The present invention provides for pooled human catalytic immunoglobulins with therapeutic utility. The prefix “CIVIG” is used to refer to pooled IgG, IgM and IgA from serum, e.g., CIVIGg, CIVIGm and CIVIGa. The prefix CIVIGas is used to designated IgA from saliva. As used herein, the terms “abzyme” or “catalytic immunoglobulin” are used interchangeably to describe at least a portion of one or more antibodies possessing enzymatic activity. Enzymatic activity includes, e.g., protease, nuclease, kinase or other like activities. As used herein, “classes” and “subclasses” refer to classes and subclasses of heavy chains and light chains. According to differences in their heavy chain constant domains, immunoglobulins are grouped into five classes: IgG, IgA, IgM, IgD and IgE. Each class of immunoglobulins can contain either κ or λ type of light chains. As used herein, the term “class selected” is used to describe the selection of one or more immunoglobulin class. Human IgG and IgA class immunoglobulins can be further subclassified into subclasses, depending on the subclass of the heavy chains. For example, IgA immunoglobulins are subclassified into two subclasses, IgA1 and IgA2. As used herein, the term “subclass selected” is used to describe the selection of one or more immunoglobulin subclass and may include all, some or one of the subclasses. For example, the IgA1 and IgA2 subclasses of IgA can be readily separated by methods known in the art using immobilized lectins such as Jacalin or immobilized antibodies directed to IgA1 and IgA2 antibodies (43, 44).

Important aspects of the invention described in this section and following examples are:

(a) Different classes of immunoglobulins express differing levels of catalytic activity. Thus, selection of immunoglobulins with the greatest activity is a useful to maximize biological benefits derived from immunoglobulin catalytic activity;

(b) Mucosal secretions often contain immunoglobulins with catalytic activity superior to immunoglobulins from blood. Thus, secretions such as saliva and milk containing immunoglobulins produced in the mucosal environment are a superior source of CIVIG preparations;

(c) Pooling of the catalytic immunoglobulins from many individual humans diversifies the range of catalysts with differing specificities and catalytic activities. The nature of immunoglobulins produced by individual humans depends on adaptive processes occurring in response to their unique immunological history (e.g., exposure to differing microbes), with the result that pooling of the immunoglobulins increases the repertoire of immunoglobulin with distinct specificity and catalytic activity directed to a larger number of antigens. Furthermore, polyclonal antibody mixtures such as the CIVIG preparations contain antibodies directed to antibodies, including antibodies directed to immunoglobulin constant domains and immunoglobulin variable domains. As the catalytic activity is subject to regulation by binding of the immunoglobulins to other immunoglobulins, pooling of immunoglobulins from different humans is expected to result in changes in the catalytic activity. This is consistent with the superior proteolytic activity observed in CIVIG preparations compared to a panel of monoclonal antibodies disclosed in Examples 1 and 2 hereunder.

(d) The CIVIG preparation method entails measurement of catalytic activity at various steps of the fractionation methods, and unlike conventional IVIG fractionation methods, CIVIG fractionation methods are designed to minimize loss of catalytic activity. Depending on the intended use of the CIVIG preparations, the catalysis assays utilize model substrates to identify promiscuous catalytic activity (e.g., Glu-Ala-Arg-aminomethylcoumarin, abbreviated EAR-MCA), or polypeptide substrates to identify specific catalytic activity. Examples of the latter class of substrates provided herein include HIV gp120 and HIV Tat. Also disclosed are examples of catalytic activity directed to Staphylococcal virulence factors such as Protein A. Further, catalytic activities directed to autoantigens are disclosed, for example, amyloid β peptides and CD4. Further, methods are disclosed for selectively fractionating the catalytic species within the CIVIG preparations, based on the reaction of electrophilic compounds with the nucleophilic sites located in the catalytic species.

### Promiscuous Catalytic Activity of Immunoglobulins

Presented here are descriptions and results observed using IgG, IgM and IgA purified from the pooled serum of 35 humans and pooled saliva of 4 humans by affinity chromatography methods (45). Additional details concerning methods and biological significance of the promiscuous catalytic activity of immunoglobulins are presented in Example 1. The IgG, IgM and IgA from serum are designated heretofore with the prefix CIVIG, corresponding, respectively, to CIVIGg, CIVIGm and CIVIGa, while IgA from saliva was designated CIVIGas. Immobilized antibody to IgA was used to purify the serum and salivary IgA. The immunoglobulins were electrophoretically homogenous and immunoblots of the gels were stainable with the appropriate antibody to IgG, IgM and IgA.

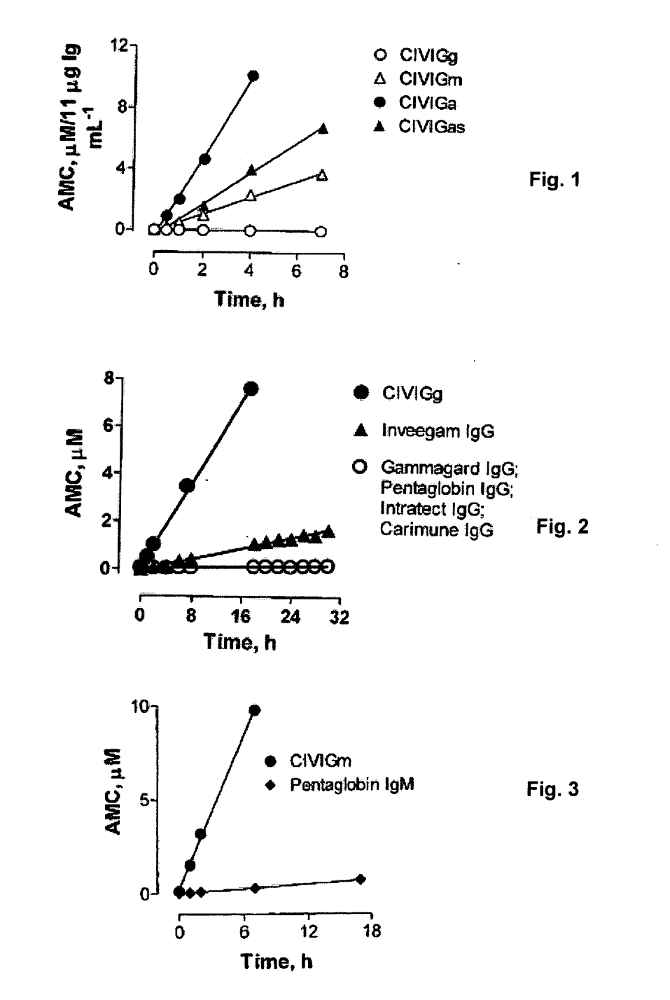

The model peptide substrate Glu-Ala-Arg-aminomethylcoumarin (EAR-AMC) was used to determine proteolytic activity of the various immunoglobulin preparations by a fluorimetric assay that measures release of aminomethylcoumarin due to cleavage of the amide bond. Of the serum immunoglobulin classes studied, the greatest catalytic activity per unit mass of the proteins was found in the CIVIGa fraction, and the CIVIGg fraction was the least active (FIG. 1). CIVIGas (salivary IgA) displayed lower activity than CIVIGa (serum IgA), but its activity was substantially greater than the serum IgG fraction. The finding of high level activity in the IgA fraction is important, as this immunoglobulin subclass is a product of mature B cells. High level catalytic activity of IgMs compared to IgGs has been reported (45). IgMs are the first products of B cells as they undergo adaptive maturation. Based on the low levels of activity of IgGs, it is suggested that improvement of the catalytic activity is disfavored event under conditions of physiological maturation of the B cells. The IgA data indicate that there is no restriction to production of improved catalytic antibodies of IgA class by mature B cells.

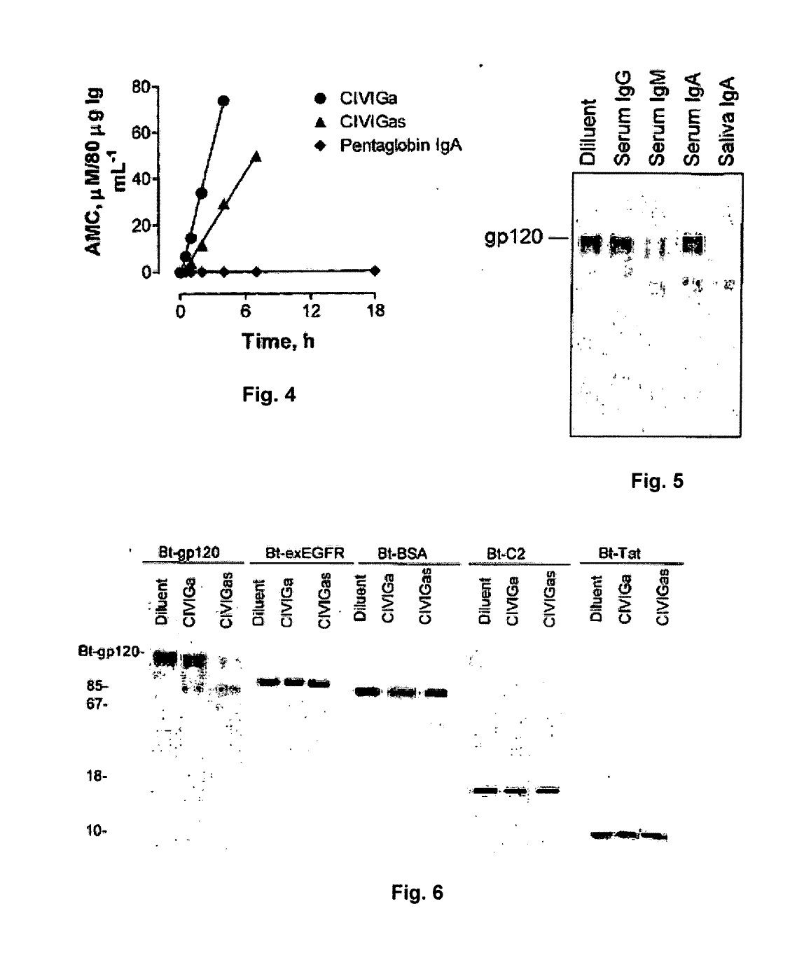

Next, the EAR-AMC cleaving activities of the CIVIGg, CIVIGm, CIVIGa and CIVIGas were compared with the corresponding IgG, IgM and IgA purified from commercial IVIG preparations, designated IVIGg, IVIGm and IVIGa. The commercial source materials were Pentaglobin (Biotest Pharma GmbH; a mixture of IgG, IgM and IgA) and Intratect (Biotest Pharma GmbH), Gammagard S/D (Baxter Healthcare Corporation), Inveegam EN (Baxter Healthcare Corporation) and Carimune NF (ZLB Bioplasma AG), all of which are IgG preparations containing only trace amounts of other immunoglobulins classes. Identical immunoaffinity procedures were employed to purify the immunoglobulins from serum or saliva (CIVIG preparations) and the commercial IVIG preparations. In the case of each immunoglobulin class, the CIVIG preparations displayed substantially greater catalytic activity than the corresponding IVIG preparations. The comparisons are shown in FIG. 2 (CIVIGg versus IgG fraction from various IVIG preparations, designated IVIGg), FIG. 3 (CIVIGm vs IVIGm) and FIG. 4 (CIVIGa and CIVIGas vs IVIGa).

The commercial IVIG preparations were also studies without further immunoaffinity purification. As shown in Table 1, CIVIGg consistently displayed greater EAR-MCA cleaving activity compared to the IgG-containing IVIG preparations. Similarly, CIVIGa, CIVIGas and CIVIGm displayed greater activity than the commercial IVIG mixture of IgG, IgM and IgA (Pentaglobin). Table 1 shows the specific EAR-AMC hydrolyzing activity of CIVIG and IVIG preparations. Catalytic activity was measured as in FIG. 1.

TABLE 1

| | Specific activity, |
|---|---|
| Ig class | nM/h/μg mL⁻¹ |
| IgG, CIVIGg | 5.94 ± 0.09 |
| IgM, CIVIGm | 38.00 ± 0.51 |
| IgA, CIVIGa | 142.34 ± 3.56 |
| IgA, CIVIGas | 91.88 ± 3.13 |
| Pentaglobin | 0.59 ± 0.03 |
| Intratect | <0.02 |
| Gammagard | 0.40 ± 0.11 |
| Inveegam | 1.28 ± 0.07 |
| Carimune | 0.77 ± 0.13 |

Table 2 shows the cleavage preference of CIVGa and CIVIGas. Reaction conditions: CIVGa and CIVIGas, 3 μg/mL; peptide-AMC substrates, 0.2 mM; 37° C. Blocking groups at the N-termini of the substrates were: succinyl, AE-AMC, AAA-AMC, AAPF-AMC, IIW-AMC; t-butoxycarbonyl, EKK-AMC, VLK-AMC, IEGR-AMC, EAR-AMC. Values (means of 3 replicates±S.D.) are the slopes of progress curves monitored for 30 h.

TABLE 2

V, μM/h/μM Ig

| Substrate | CIVIGa | CIVIGas |
|---|---|---|
| AE-AMC | <0.18 | <0.18 |
| AAA-AMC | <0.18 | <0.18 |
| IIW-AMC | <0.18 | <0.18 |
| AAPF-AMC | <0.18 | <0.18 |
| EKK-AMC | <0.18 | 3.9 ± 1.7 |
| VLK-AMC | 0.7 ± 0.0 | 6.1 ± 0.1 |
| EAR-AMC | 28.2 ± 2.1 | 41.1 ± 1.7 |
| IEGR-AMC | 0.5 ± 0.3 | 24.2 ± 0.5 |
| PFR-AMC | 0.5 ± 0.0 | 53.2 ± 0.8 |
| GP-AMC | <0.18 | 2.7 ± 0.2 |
| GGR-AMC | 1.1 ± 0.2 | 45.5 ± 0.7 |
| GGL-AMC | <0.18 | <0.18 |

CIVIGa and CIVIGas displayed greatest cleavage of peptide substrates containing a basic residue on the N terminal side of the scissile bond (Table 2). However, differences in the fine specificity of CIVIGa and CIVIGas were evident, with the latter showing less strict flanking residue requirements. The superior activity of the CIVIG preparations can be attributed to the comparatively gentle method of isolating the immunoglobulins from serum and saliva. i.e., immunoaffinity chromatography. To the extent that the catalytic function of immunoglobulins can result in a superior therapeutic effect, the CIVIG preparations are more suitable than commercial IVIG preparations for clinical use.

### Catalytic Immunoglobulins Capable of Cleaving Polypeptides

The ability of IgM antibodies from uninfected humans to selectively catalyze the cleavage of the HIV-1 coat protein gp120 has been described (46). The CIVIGa and CIVIGas preparations displayed dose dependent cleavage of biotinylated gp120, evident as depletion of the intact gp120 band in electrophoresis gels and appearance of lower mass fragments of the protein. Each of serum IgA and salivary IgA from four humans displayed the gp120 cleaving activity, confirming the widespread distribution of the catalytic IgAs. FIG. 5 illustrates the cleavage activity of gp120 by diluent, CIVIGas, CIVIGg, CIVIGm and CIVIGa from pooled human blood and pooled human saliva. Biotinylated gp120 was incubated with the serum IgG, IgM, and IgA and saliva IgA and the reaction mixtures were visualized by reducing SDS-electrophoresis. From dose response curves, the average activity of salivary IgA was ˜20-fold greater than of serum IgA.

Table 3 illustrates superior gp120 hydrolyzing activity of saliva IgA compared to serum IgA, normalized to mg Ig/mL. IgG was poorly catalytic. IgG purified from commercial IVIG also displayed no detectable gp120 cleaving activity (Intratect IVIGg, Pentaglobin IVIGg; 150 μg/mL, assayed as in FIG. 5). Similarly the Intratect IVIG and Pentaglobin IVIG without fractionation by immunoaffinity chromatography failed to cleave gp120 (150 μg/mL). Gp120 cleavage activity was measured with serum IgA, 144 μg/mL and saliva IgA, 32 μg/mL as in FIG. 5, and the activity was normalized to mg Ig/mL.

TABLE 3

| | Specific activity, nM/h/mg mL⁻¹ | | Relative activity |
|---|---|---|---|
| IgA preparation | Serum | Saliva | (Saliva/Serum) |
| 2288 | 5.1 ± 1.7 | 131.0 ± 17.4 | 26 |
| 2289 | <4.5 | 96.2 ± 0.8 | >21 |
| 2290 | <4.5 | 134.0 ± 14.5 | >30 |
| 2291 | 8.9 ± 1.3 | 152.8 ± 15.3 | 17 |

The catalytic activity of CIVIGa and CIVIGas was gp120-selective, evident from undetectable cleavage of several unrelated proteins. This is illustrated in FIG. 6. Biotinylated proteins studied in this FIG. 6 were gp120, the extracellular domain of epidermal growth factor receptor (exEGFR), bovine serum albumin (BSA), the C2 domain of human coagulation factor VIII (C2), and HIV-Tat.

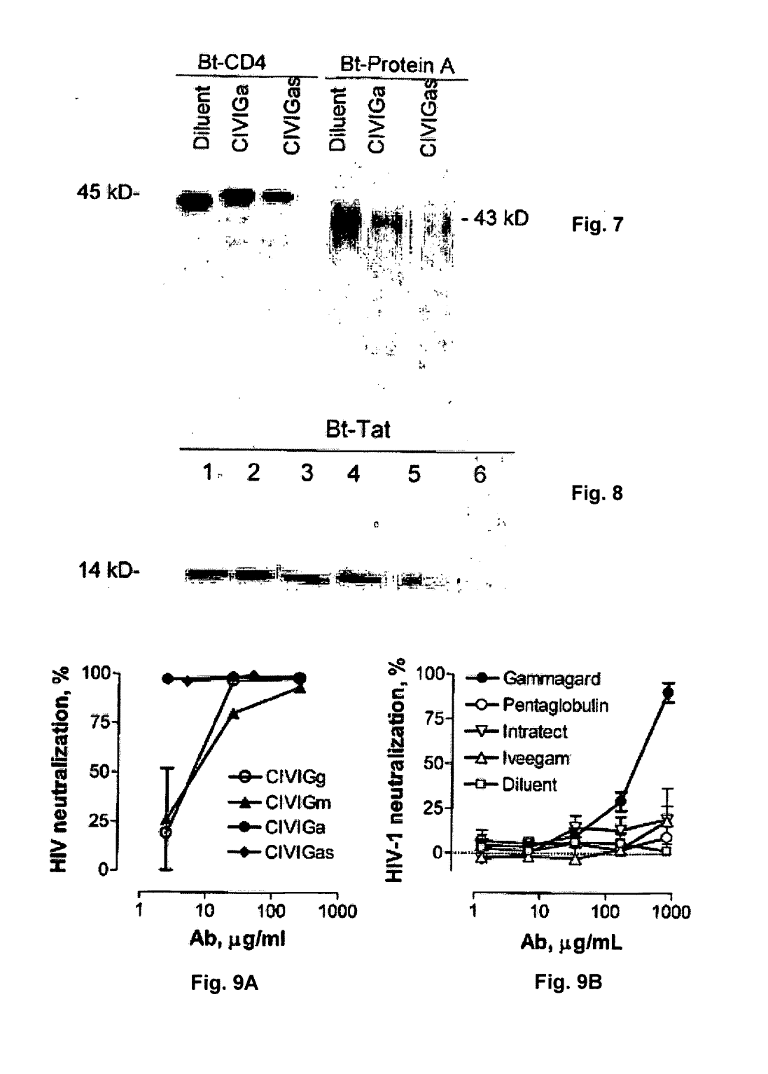

Further studies revealed that the CIVIGa and CIVIGas preparations can also cleave certain other proteins. In particular, FIG. 7 displays the cleavage of Protein A, and to a lesser extent, CD4, by these preparations. Protein A is a staphylococcal protein previously described to bind immunoglobulins as a superantigen (47). Certain monoclonal IgMs analyzed previously were devoid of protein A cleaving activity (46). The Protein A employed in these studies was iodinated prior to biotinylation to inactivate the Fc binding site, while leaving intact the recognition as a superantigen by the V domains. The IgA catalyzed hydrolysis of protein A may be attributed an adaptive improvement of the catalytic site over the course of B cell differentiation. With respect to CD4 cleavage, the presence of CD4 binding antibodies in patients with autoimmune disease and HIV infection has been reported, and a commercial IVIG preparation also contains CD4 binding antibodies. The CD4 cleavage by CIVIGa and CIVIGas indicates that a subpopulation of antibodies that bind CD4 can proceed to catalyze the cleavage of this protein.

Further study of the HIV protein Tat indicated the catalytic hydrolysis of this protein by CIVIGm but not CIVIGa, CIVIGas or CIVIGg. This is illustrated in FIG. 8, evident by depletion 14-kD band in electrophoresis gels. It can be concluded that different classes of immunoglobulins hydrolyze various polypeptide to differing extent. Previously, IgM antibodies from uninfected humans were described to bind Tat. Thus, the failure of CIVIGa and CIVIGas, which were derived from uninfected humans, to hydrolyze Tat may be interpreted as reflecting the absence of an endogenous antigen that drives B cell maturation. In comparison, the efficient cleavage of gp120 by CIVIGa and CIVIGas can be explained by the presence of an endogenous antigen with sequence identity to the superantigenic region of gp120 that might induce IgA class antibody responses in uninfected humans.

FIG. 9A illustrates findings that CIVIGa and CIVIGas neutralized the infection of cultured peripheral blood mononuclear cells (PBMCs) by a primary CCR5-coreceptor dependent HIV strain (ZA009) potently. The HIV-1 preparation was incubated with CIVIG preparations and commercial IVIGs at varying concentrations, then allowed to infect PBMC and the extent of infection determined by measuring capsid protein p24 levels. HIV-1 neutralization activity is expressed as percent decrease of p24 concentrations as compared to treatment with diluent (phosphate-buffered saline; PBS). CIVIGm and CIVIGg displayed lower potency neutralizing activity. Several commercial IVIG preparations were devoid of detectable neutralizing activity, but one IVIG preparation (Gammagard) displayed low-level activity (FIG. 9B).

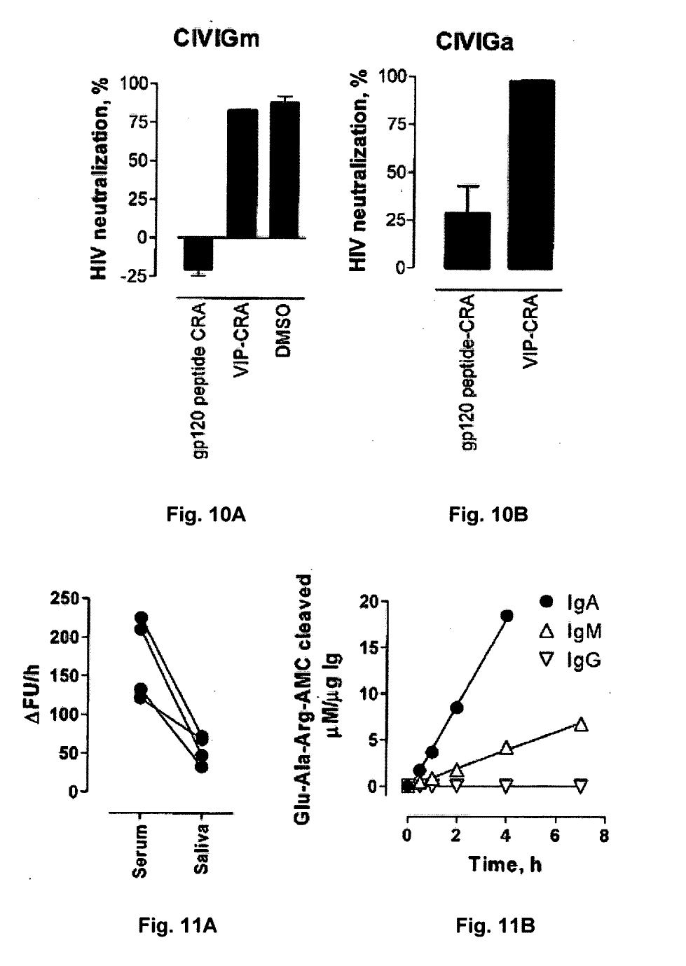

Covalently reactive analogs (CRAs) of polypeptides have been developed as probes for antibodies. CRAs contain an electrophilic phosphonate analog capable of irreversible binding to nucleophiles present in antibody combining sites. The covalent reaction occurs in coordination with noncovalent antigen-antibody binding, ensuring specificity, and permitting the use of peptidyl CRAs for irreversible and specific binding to the antibodies. One such peptidyl CRA reported is an analog of gp120 residues 421-433 containing the phosphonate at its C terminus (gp120 peptide CRA). This region of gp120 is a component of the superantigenic site of this protein. Neutralization of HIV-1 by CIVIGa and CIVIGm was inhibited by the gp120 peptide CRA, confirming that recognition of the gp120 superantigenic site is required for the neutralizing activity. FIGS. 10A-10B illustrate these findings. CIVIGm and CIVGa were preincubated with the gp120 peptide-CRA or diluent, and the residual neutralization activity was determined as in FIG. 9. An irrelevant peptide CRA, VIP-CRA was employed as the control reagent to rule out nonspecific effects. The neutralizing activity of both CIVIG preparation tested was reduced in the presence of the gp120 peptide CRA. Taken together, these findings indicate that pooled polyclonal immunoglobulins with catalytic activity can neutralize HIV by recognizing the superantigenic site of gp120.

### CIVIG Utility

Assuming equivalent effector functions residing in the constant domains, the chemical transformation of antigens by catalytic antibodies can be anticipated to exert biological effects superior to ordinary antibodies. First, the catalytic reaction entails chemical transformation of the antigen, which results in permanent changes in the bioactivity of the antigen. Dissociation of antigen from reversibly-binding antibodies, in contrast, regenerates antigen with unmodified bioactivity. Second, catalysts are capable of turnover, i.e., a single catalyst molecule can chemically transform multiple antigen molecules. In comparison, ordinary antibodies act stoichiometrically, e.g., an IgG, IgM and secretory IgA bind at most 2, 10 and 4 antigen molecules, respectively. Comparatively large amounts of conventional IVIG preparations are administered for the therapy of various diseases, e.g., 1 g/kg body weight with the treatment repeated at monthly intervals (48). Depending on the rate of catalysis displayed by the CIVIG formulation, comparatively small CIVIG amounts are predicted to be efficacious therapeutic agents.

For example, the relative therapeutic efficacy of IVIG and CIVIG preparations may be predicted under the following assumptions: (a) antigen binding and antigen catalytic cleavage are the mechanisms of the therapeutic effects of IVIG and CIVIG, respectively, and (b) the pharmacokinetics of IVIG and CIVIG preparation are equivalent. If the CIVIG preparation displays a catalytic rate constant of about 2 moles antigen/mole immunoglobulin/min (this is close to the observed rate constant for certain CIVIG preparations), 20,160 moles antigen are hydrolyzed/mole CIVIG over 7 days. If it is further assumed that 10% of the CIVIG preparation consists of catalytic immunoglobulins and 10% of the IVIG preparation consists of antigen-binding immunoglobulins, it can be deduced that the one mole of bivalent IVIG will at best bind 0.2 moles antigen. Under these assumptions, the therapeutic efficacy of the CIVIG preparation will be about 100,000-fold greater than IVIG, and administration of 10 μg CIVIG/kg body weight will yield equivalent therapeutic benefit to 1 gram IVIG/kg body weight at the end of 7 days. These assumptions are obviously oversimplified for illustrative purposes, and in reality, the relative benefit of the preparations must be determined empirically.

In principle, any disease in which removal of an antigen by catalytic antibodies is open to therapy using CIVIG preparations. The skilled artisan will recognize that there are a variety of diseases that can be treated by the present invention. Useful therapeutic applications are predictable both for promiscuous catalytic antibodies (Example 1) as well as antigen specific catalytic antibodies (Examples 2 and 3). IVIG has been used in the literature for treatment of several diseases, and its use in additional diseases is under considerations and understood by a person of skill in the art. The therapeutic use of CIVIG preparations in all of these medical conditions can be foreseen, e.g., autoimmune thrombocytopenic purpura, systemic lupus erythematosus, anti-phospholipid syndrome, vasculitis, inflammatory myositis, rheumatoid and juvenile chronic arthritis, Alzheimer's disease, bacterial infections, septic shock, HIV infection, and organ and cell transplants.

Conventional IVIG is generally very well-tolerated. The commonest side effects are flu-like symptoms, which can be managed by stopping infusion temporarily or prior hydrocortisone administration. Therefore, there is no reason to expect that the side effects of CIVIG preparations will be intolerable. In IgA-deficient individuals, due to the possibility of anaphylaxis, the use of CIVIGa and CIVIGas formulations is contraindicated.

### Route of Administration

The usual route of administration of IVIG is into the blood via intravenous injections, and CIVIG administration by this route is also predicted to exert therapeutic effects. Formulation of the CIVIG in physiological saline along with suitable excipients known in the art is suitable for administration by the intravenous route. Other routes are anticipated to be useful in certain situations and are known to the skilled artisan. In the case of HIV infection, administration of the CIVIG as a gel or another suitable formulation by the vaginal or rectal route is predicted to protect against vaginal and rectal transmission of the virus. For semantic clarity, the CIVIG formulations will more properly be designated in these applications as catalytic intravaginal immunoglobulins and catalytic intrarectal immunoglobulins. For skin diseases, topical applications of the CIVIG formulations is appropriate. In each of these applications, appropriate excipients will be incorporated into the formulation For example, a suitable formulation of CIVIG for vaginal application is as a gel in hydroxyethylcellulose (e.g., 2.5% hydroxyethyl cellulose gel, Natrosol 250HHX Pharm, Hercules/Aqualon). This gel is used as an inert carrier for several vaginal microbicides under development. The concentration of the gel base will be appropriate to obtain sufficient rate of spreading in the genital tract and appropriate applicators will be employed to deposit the gel in the vagina a few minutes prior to sexual intercourse, e.g., 5 minutes.

### Source

The preferred CIVIG formulations are derived from a random collection of serum or plasma donated by humans at blood banks after appropriate exclusion of individuals with transmissible infections. IgA, IgM and IgG concentrations in serum or plasma are, respectively, about 3, 1.5 and 12 g/liter. For certain target diseases, more restrictive criteria can be applied. For example, for Alzheimer's disease, the blood collection can be biased towards inclusion of older subjects, as the amyloid peptide antibodies tend to increase with advancing age. Similarly, blood from HIV infected individuals can be the preferred source of CIVIG preparations, as the infection can be associated with increased proteolytic antibodies to the virus. Milk is another source of CIVIG, as IgA concentrations in milk are comparatively high (colostrum and mature milk, respectively, about 12 and 1 g/liter). Saliva from human donors is a convenient source of CIVIGas, which contains high levels of proteolytic HIV antibodies. IgA concentrations in saliva are about 0.3 g/liter). Large amounts of the saliva (e.g., about 20 ml) can be readily collected within a few minutes in a non-invasive manner, e.g., following stimulation of the salivary glands by chewing a small piece of parafilm for 2-3 minutes. As the antigen neutralizing potency of CIVIG preparations is superior to conventional IVIG preparations, smaller amounts of the starting material (blood, saliva, milk) are needed for to obtain therapeutic amounts of CIVIG compared to conventional IVIG. To ensure sufficient antibody diversity, it is preferable to pool the blood, saliva or milk as the case may be from many humans, e.g., 100 or more humans.

### Method of Preparation

As noted above, conventional IVIG preparations involve harsh treatments with organic solvents. Moreover, most marketers of IVIG have focused on immunoglobulins of the IgG class, which possesses substantially lower catalytic activity compared to the IgA and IgM classes. Consequently, the CIVIG formulations are the CIVIGm and CIVIGa (and CIVIGas) in many cases. Immunoaffinity methods are suitable for one-step purification of the CIVIG preparations from blood and mucosal fluids like saliva. For CIVIGg, immobilized antibody to IgG or bacterial IgG-binding proteins (e.g., protein G) can be employed for purification. For CIVIGm and CIVIGa (and CIVIGas), immobilized antibodies to human IgM and anti-IgA are suitable and yield electrophoretically homogeneous immunoglobulins. If needed, further purification can be done using appropriate fractionation procedures (e.g., chromatography, precipitation) taking care to maintain the integrity of the catalytic sites. Scale-up of the purification using immunoaffinity methods is unproblematic providing the stoichiometry of the immunoglobulins and the immunoglobulin binding matrix is maintained at optimal levels. The recovered immunoglobulins are concentrated to the desired concentration by ultrafiltration or freeze-drying methods.

An alternative method to obtain CIVIG preparations is to employ chromatography matrices that enrich for the catalysts of interest. For example, matrices containing certain proteins in an immobilized form can be deduced to be useful for this purpose, e.g., Protein A and Protein L. These proteins bind the superantigen binding sites of the antibodies, and recovery of highly catalytic immunoglobulins is anticipated because of the favorable molecular interrelationship between catalysis and superantigen binding.

Ligands with the ability to bind the catalytic site preferentially are another alternative for CIVIG purification. For example, the extent of the reaction with covalently reactive analogs (CRAs) containing electrophiles predicts which antibodies have the greatest catalytic activity (49). Hapten CRAs or polypeptide CRAs can be employed to isolate promiscuous CIVIG and antigen-selective CIVIG, respectively, by allowing the covalent reaction to proceed on a solid phase, followed by elution of enriched catalysts using reagents that cleave the phosphonate ester linkage to the antibody nucleophile, e.g., pyridinium aldoxime reagents (50).

Ways to protect the catalytic site during purification can also be foreseen, which are useful to obtain CIVIG preparations using conventional IVIG purification methods that are comparatively harsh and may otherwise denature the catalytic site. For example, conventional IVIG is prepared using the cold ethanol precipitation procedure entailing variations in solvent temperature. The inclusion of a polypeptide VIP during purification of a catalytic immunoglobulin light chain entailing a denaturation-renaturation cycle using guanidine hydrochloride permits recovery of superior catalytic activity (51). Thus, inclusion of excess peptide substrate during employed conventional IVIG preparation can yield high activity CIVIG preparations. Similarly, inclusion of CRAs during conventional IVIG preparation may allow recovery of high activity CIVIG preparations, as the catalytic site will be frozen into its active state once the electrophile binds covalently to the immunoglobulin nucleophile. The immunoglobulin-CRA complexes are then treated with hydroxylamine or a pyridinium aldoxime reagent (26) that is known to disrupt the covalent bond between the antibody nucleophile and the electrophile in the CRA. Following removal of the dissociated CRA product (e.g., by dialysis), the CIVIG can be recovered in active form.

### Example 1

### Amidolytic IgAs

Abbreviations used: Ab, antibody; AMC, 7-amino-4-methylcoumarine; CHAPS, 3-[(3-cholamidopropyl)dimethylammonio]-1-propanesulfonic acid; DFP, diisopropyl fluorophosphates; FU, fluorescence unit; SDS, sodium dodecylsulfate.

The secreted antibody (Ab) repertoire is generated from programmed expression of the constant (μ, δ, γ, α, ε) and variable domain genes (V, D, J genes). The IgG and IgA Ab classes are the dominant products of mature B lymphocytes responsible for adaptive immunological defense against microbial infections. Abs from healthy individuals catalyze diverse chemical reactions. Polyclonal and monoclonal IgMs, the first Ab class produced in the course of B cell differentiation, can ubiquitously hydrolyze model tripeptide and tetrapeptide substrates. The activity is promiscuous in regard to the peptide sequence requirements, limited only by the requirement for a positive charge neighboring the scissile amide bond in the model substrates, and is characterized by low affinity recognition of the substrate ground state. The Ab-catalyzed reaction occurs via a serine peptidase-like nucleophilic mechanism, indicated by inhibition of catalysis by electrophilic phosphonate diesters that were originally developed as irreversible inhibitors of serine proteases such as trypsin.

Very little is known about the developmental aspects of Ab catalysis. Noncovalent occupancy of the B cell receptor (BCR, membrane bound Ig complexed to signal transducing proteins) by antigens is well-known to drive the clonal selection of B cells, resulting eventually in production of mature IgG, IgA and IgE Abs capable of specific binding to individual polypeptide antigens. Examples of mature IgGs with catalytic activity have been reported, particularly in autoimmune diseases. However, production of antigen-specific catalytic IgGs under normal circumstances is a rare event, and the IgGs generally hydrolyze the model peptide substrates at levels considerably lower than IgMs. This suggests that catalytic hydrolysis of peptide antigens by IgG-type BCRs is an immunologically disfavored event. It has been difficult until now, therefore, to conceive of Ab catalysis as a mechanism of adaptive immunological defense against microbial infection. Similarly, despite extensive previous attempts (e.g., by immunization with analogs of the antigen ground state and transition state), catalytically efficient monoclonal IgGs to clinically important antigens have not been developed (52). The difficulties may be explained by an intrinsic deficiency in the catalytic power of IgG class Abs.

IgAs are commonly thought to function as defense mediators against microbial infection at mucosal surfaces. Like IgGs, IgAs are produced by terminally differentiated B cells. Recent studies have shown that IgAs in human milk and sera of patients with multiple sclerosis display kinase and protease activities (53) Objective comparisons of the catalytic efficiencies of IgA, IgG and IgM Abs, however, are not available. It is demonstrated herein that IgAs isolated from the blood and saliva of healthy humans catalyze the cleavage of model peptide substrates with efficiency considerably superior to that of IgGs. This finding highlights the IgA compartment of the humoral immune response as a source of natural catalysts and raises interesting questions concerning the immunological mechanisms favoring catalytic antibody synthesis.

### Antibody Preparations

Polyclonal Abs were purified from the serum derived from peripheral venous blood or saliva of 4 humans subjects without evidence of infection or immunological disease (1 female and 3 males; age 28-36; our laboratory identification codes, 2288-2291). Saliva was obtained following chewing of parafilm for 2 min (16). The Abs were also analyzed as pools prepared from the individual IgA, IgG and IgM fractions purified from 34 humans subjects without evidence of disease (17 females and 17 males; age 17-65; white 30, black 2, Asian 2; identification codes 679, 681-689 and 2058-2081; Gulf Coast Blood Bank). Protocols related to blood and saliva collection were approved by the Univ of Texas Committee for Protection of Human Subjects and informed consent was obtained from the human donors.

For IgA purification, the serum (0.5 mL) was incubated with goat anti-human IgA agarose (1 h, 1 mL settled gel in a Poly-Prep chromatography column (Bio-Rad) with rotation; Sigma-Aldrich; St. Louis, Mo.) in 50 mM Tris.HCl, pH 7.5, containing 0.1 mM CHAPS. The unbound fraction was recovered and the gel washed with 50 mM Tris.HCl, pH 7.5, containing 0.1 mM CHAPS (4 mL×5). Bound IgA was eluted with 0.1 M glycine, pH 2.7, containing 0.1 mM CHAPS (2×2 mL), into collection tubes containing 1 M Tris.HCl, pH 9.0 (0.11 mL/tube). Monoclonal IgAs were purified in the same manner from the sera of patients with multiple myeloma (Dr. Robert Kyle, Mayo Clinic, identification codes, 2573-2587) or from commercially available human IgA preparations (also isolated from multiple myeloma patients; 2 IgA1 preparations, catalog #BP086 and BP087; 2 IgA2 preparations, catalog #BP088 and BP089; Binding Site Inc, San Diego, Calif.). Salivary IgA was purified similarly (7 mL saliva, 0.5 mL anti-IgA settled gel). IgG and IgM were purified on protein G-Sepharose and anti-IgM-agarose columns, respectively, using as starting materials the unbound fractions from the anti-IgA columns as described previously (54).

Protein concentrations of purified Ab samples were determined using a microBCA kit (Pierce). SDS-electrophoresis gels were immunoblotted with peroxidase-conjugated goat anti-human α, anti-human λ, anti-human κ, and anti-secretory component Abs (Sigma-Aldrich). Gel filtration of serum and salivary IgA (1.6 mg and 0.8 mg; purified from identification codes 2288-2291) was in 6 M guanidine hydrochloride (Sigma-Aldrich), pH 6.5, on a Superose-6 FPLC column (Pharmacia) essentially as in previous studies (54). Nominal mass of proteins of the A280 peaks in the eluent was determined by comparison with the retention volumes of monoclonal IgM CL8702 (900 kD; Cedarlane), thyroglobulin (330 kD; Calzyme Laboratories) and human myeloma IgG3, λ (150 kD; Sigma-Aldrich). The monomer fractions from serum IgA (corresponding to retention volume of 10.8-11.4 mL) were pooled and dialyzed against 50 mM Tris.HCl-0.1 M glycine, pH 8.0, containing 0.1 mM CHAPS at 4° C. (2 L×5) for 4 days prior to assay for amidolytic activity.

### Amidolytic Activity

Substrates used are 7-amino-4-methylcoumarin (AMC) conjugates of: Boc-Glu(O-Bzl)-Ala-Arg (Boc, tert-butoxycarbonyl; Bzl, benzyl; Glu-Ala-Arg-AMC); Suc-Ala-Glu (Suc, succinyl; Ala-Glu-AMC); Suc-Ala-Ala-Ala (Ala-Ala-Ala-AMC); Suc-Ile-Ile-Trp (Ile-Ile-Trp-AMC); Suc-Ala-Ala-Pro-Phe (Ala-Ala-Pro-Phe-AMC) (SEQ ID NO: 4); Boc-Glu-Lys-Lys (Glu-Lys-Lys-AMC); Boc-Val-Leu-Lys (Val-Leu-Lys-AMC); Boc-Ile-Glu-Gly-Arg (Ile-Glu-Gly-Arg-AMC) (SEQ ID NO: 5); Pro-Phe-Arg (Pro-Phe-Arg-AMC); Gly-Pro (Gly-Pro-AMC); Z-Gly-Gly-Arg (Z, benzyloxycarbonyl; Gly-Gly-Arg-AMC); Z-Gly-Gly-Leu (Gly-Gly-Leu-AMC) (Peptides International, Louisville, Ky. or Bachem, King of Prussia, Pa.). Hydrolysis of the amide bond linking AMC to the C-terminal amino acid in the substrates was measured in 50 mM Tris.HCl-0.1 M glycine, pH 7.7, containing 0.1 mM CHAPS at 37° C. in 96-well plates by fluorimetry (λex 360 nm, λem 470 nm; Varian Cary Eclipse). Authentic AMC (Peptides International) was used to construct a standard curve.

In inhibition studies, Glu-Ala-Arg-AMC (0.4 mM) was incubated with IgA (8 μg/mL; from identification code 2288) in the presence or absence of diisopropyl fluorophosphate (DFP; Sigma_Aldrich) or diphenyl N-(6-biotinamidohexanoyl)amino(4-amidinophenyl)methanephosphonate (1a; prepared as in ref 55) and the AMC fluorescence monitored as described above. Stoichiometry of inhibition was estimated as follows. Monoclonal IgA (1.6 mg/mL; from identification code 2582) was incubated with diphenyl N-(benzyloxycarbonyl)amino(4-amidinophenyl)methanephosphonate 1b (2.5-20 μM; prepared as in ref 56) at 37° C. in 50 mM Tris.HCl-0.1 M glycine, pH 7.7, containing 0.1 mM CHAPS and 0.5% dimethylsulfoxide. After 18 h, the residual activity was measured by incubating the 1b-treated IgA (24 μg/mL) with Glu-Ala-Arg-AMC (0.4 mM).

### Phosphonate Binding

Purified IgA Abs (160 μg/mL; from identification code 2288) were treated with phosphonate diesters 1a or 2 (0.1 mM; 2 prepared as described in 20) in 10 mM phosphate buffered saline, pH 7.1, containing 0.1 mM CHAPS at 37° C. for 6 h. Formation of phosphonate-Ab adducts was determined by SDS-electrophoresis followed by streptavidin-peroxidase staining of the blots as described previously (57, 58).

### Amidolytic Activity of IgA.

The catalytic activity of IgA samples from 4 healthy human sera and saliva was initially screened for hydrolysis of Glu-Ala-Arg-AMC. Serum IgG and IgM isolated from healthy humans have previously been shown to hydrolyze this substrate. Cleavage of the amide bond linking Arg and the coumarin moiety in the substrate serves as a convenient surrogate for peptide bond hydrolysis. Background hydrolysis of the substrate incubated in buffer was negligible (<0.1 ΔFU/h). Every IgA sample screened was positive for this activity. Hydrolysis by the serum IgA fractions proceeded at rates somewhat greater than the salivary IgA fractions from the same donor (1.8-4.5-fold; FIG. 11A).

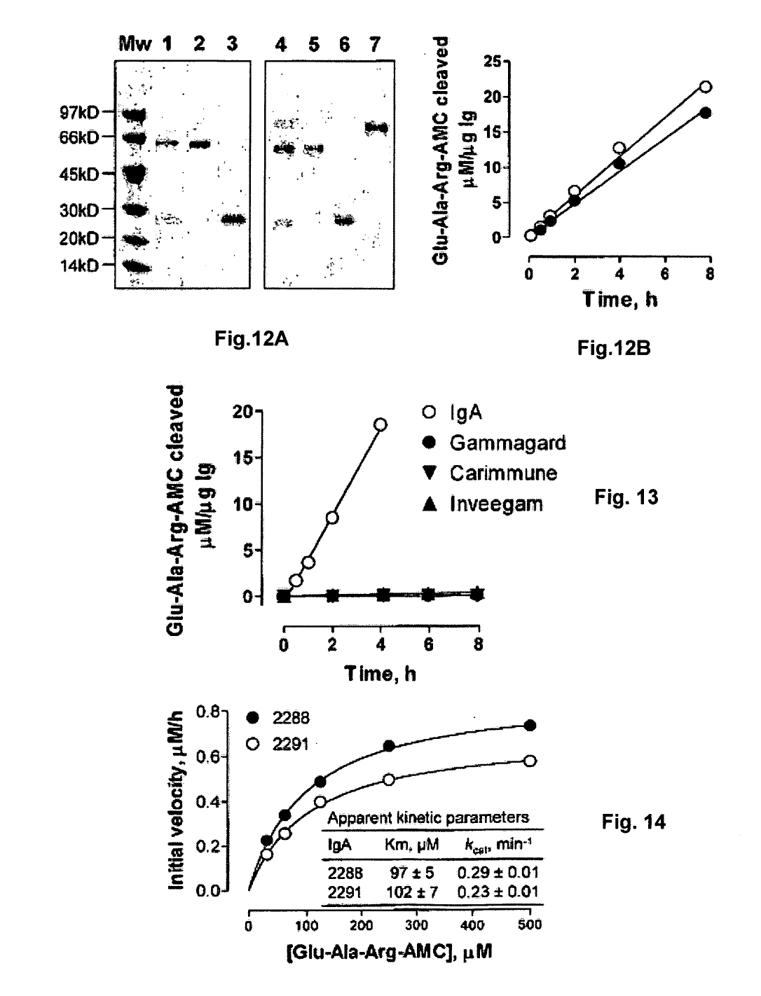

Next, the amidolytic activity of IgA, IgG and IgM fractions purified from a pool of sera from 34 healthy humans was measured. Increasing concentrations of IgA, IgG and IgM were initially employed to determine concentrations yielding measurable fluorescence signals (not shown). The observed velocities were expressed per μg Ab mass (FIG. 11B). As the combining site/mass ratio for the three Ab classes is nearly equal (2 sites/150-170 kD), this permits comparison of their amidolytic activities. IgA displayed activity 886-fold greater than the IgG (IgA and IgG, respectively, 4.70±0.15 and 0.0053±0.0003 μM substrate/h/μg Ig). Consistent with previous reports (54), readily detectable IgM catalytic activity was also evident (0.99±0.32 μM substrate/h/μg Ig). The purity of the IgG and IgM obtained by the affinity chromatography method used here has been reported previously (54). Reducing SDS-electrophoresis of serum IgA obtained by affinity chromatography revealed two protein bands with nominal mass 60 and 25 kD that were stainable with anti-α and anti-κ/λ Abs, respectively (FIG. 12A). In the salivary IgA preparation, an additional band stainable with anti-secretory component Ab was observed (85 kD). All of the bands detected by coomassie blue staining were also stainable by anti-α, anti-κ/λ or anti-secretory component Abs. None of the coomassie blue stainable bands were stainable by anti-μ or anti-α Abs.

IgAs can form noncovalent and S—S bonded multimers. The serum and salivary IgA preparations were analyzed by FPLC-gel filtration in a denaturing solvent (6 M guanidine hydrochloride) by methods employed previously to validate IgG and IgM catalytic activities (54). Consistent with previous reports, 82% and 10% of the serum and salivary IgA, respectively, was recovered as the monomer species (170 kD), and 18% and 68% was recovered as the dimer species (330 kD and 409 kD, respectively; the remaining IgA in salivary IgA sample was recovered in the large mass region, >600 kD). All IgA fractions recovered from the column displayed reducing SDS-gel electrophoresis profiles essentially identical to those in FIG. 12A. Next, the monomer IgA from serum obtained by gel filtration in guanidine hydrochloride was renatured by dialysis. The renatured IgA and the affinity-purified IgA loaded on the gel filtration column displayed near-equivalent Glu-Ala-ArgAMC cleaving activity (FIG. 12B), fulfilling the test of purification to constant specific activity. As the two preparations displayed identical activity levels, the affinity-purified IgA preparations were employed in subsequent catalysis assays without denaturing gel filtration.

Several preparations of pooled human IgG are marketed for intravenous infusion in the therapy of certain diseases (IVIG; 24-26). Like the pooled human IgG prepared in our laboratory, three commercial IVIG preparations displayed very low level cleavage of Glu-Ala-Arg-AMC compared to the pooled IgA (FIG. 13; Gammagard S/D and Inveegam EN from Baxter, respectively, 0.0012±0.0002 and 0.0432±0.0006 μM/h/μg Ig; Carimune NF from ZLB Bioplasma AG, 0.0016±0.0002 μM/h/μg Ig; these IgG preparations contain only trace amounts of IgM and IgA).

Typical enzymatic kinetics were observed in study of reaction rates for 2 IgA preparations at increasing Glu-Ala-Arg-AMC concentrations (FIG. 14). The rates were saturable at excess substrate concentration and consistent with the Michaelis-Menten-Henri kinetics. Observed Kₘ values were in the high micromolar range. These values are in the same range as reported previously for polyclonal human IgGs.

To study the extent to which the amidolytic activity varies in individual Abs, 19 monoclonal IgAs purified from the serum of patients with clinically diagnosed multiple myeloma (n=19), including 4 IgAs with known subclass (2 each belonging to subclass IgA1 and IgA2) were examined. All 19 IgAs displayed detectable Glu-Ala-Arg-AMC cleavage (Table 4). The catalytic activity varied over a 19-fold range in this panel of IgAs. The activity was detected in both IgA subclasses (2 IgA1 preparations, vendor catalog #BP086 and BP087, 38.1±8.8 and 23.8±3.4 FU/23 h, respectively; 2 IgA2 preparations, vendor catalog #BP088 and BP089, 48.9±1.0 and 50.5±3.9 FU/23 h, respectively). Table 4 shows hydrolysis of EAR-AMC by human monoclonal IgA.

TABLE 4

ΔFU

| N | Range | Median | Mean | SD |
|---|---|---|---|---|
| 19 | 20.3-393.6 | 85.7 | 101.6 | 90.5 |

IgA (8 μg/mL) was incubated with the substrate (0.4 mM) for 23 h and AMC fluorescence was measured. N represents the number of monoclonal IgA samples analyzed. Each sample was assayed in triplicates.

### Substrate Selectivity

Substrate selectivity of the polyclonal IgA preparations from serum and saliva was studied using a panel of 12 peptide-AMC conjugates (Table 5). The greatest levels of hydrolysis by the serum and saliva IgA samples occurred at the Arg-AMC bond, suggesting preferential recognition of the Arg side chain. The Lys-AMC bond in certain substrates was hydrolyzed, but at a rate lower than Arg-AMC. No hydrolytic activity on the C terminal side of acidic or neutral residue was evident, with the exception that salivary IgA displayed low-level cleavage of Gly-Pro-AMC. The preference for a basic residue at the cleavage site was also evident by comparing the cleavage of Gly-Gly-Arg-AMC and Gly-Gly-Leu-AMC cleavage, which are identical except for the Arg-AMC/Leu-AMC linkage. Interestingly, serum and salivary IgA did not hydrolyze various substrates at identical rates. The serum IgA displayed a pronounced preference for Glu-Ala-Arg-AMC whereas salivary IgA cleaved Glu-Ala-Arg-AMC, Ile-Glu-Gly-Arg-AMC (SEQ ID NO: 5), Pro-Phe-Arg-AMC and Gly-Gly-Arg-AMC at comparable rates. Table 5 shows the cleavage preference of IgA Abs from serum and saliva. Reaction conditions are: IgA, 3 μg/mL; substrates, 0.2 mM; 37° C. Values are the slopes of progress curves monitored for 30 h (means±SD of three replicates).

TABLE 5

V, μM/h/μg IgA

| Substrate | Serum | Salivary |
|---|---|---|
| Ala-Glu-AMC | <0.02 | <0.02 |
| Ala-Ala-Ala-AMC | <0.02 | <0.02 |
| Ile-Ile-Trp-AMC | <0.02 | <0.02 |
| Ala-Ala-Pro-Phe-AMC | <0.02 | <0.02 |
| Glu-Lys-Lys-AMC | <0.02 | 0.2 ± 0.1 |
| Val-Leu-Lys-AMC | 0.09 ± 0.0 | 0.31 ± 0.0 |
| Glu-Ala-Arg-AMC | 3.53 ± 0.3 | 2.12 ± 0.1 |
| Ile-Glu-Gly-Arg-AMC | 0.06 ± 0.0 | 1.24 ± 0.0 |
| Pro-Phe-Arg-AMC | 0.06 ± 0.0 | 2.73 ± 0.0 |
| Gly-Pro-AMC | <0.02 | 0.14 ± 0.0 |
| Gly-Gly-Arg-AMC | 0.14 ± 0.0 | 2.33 ± 0.0 |
| Gly-Gly-Leu-AMC | <0.02 | <0.02 |

Reactivity with Serine Protease Inhibitors

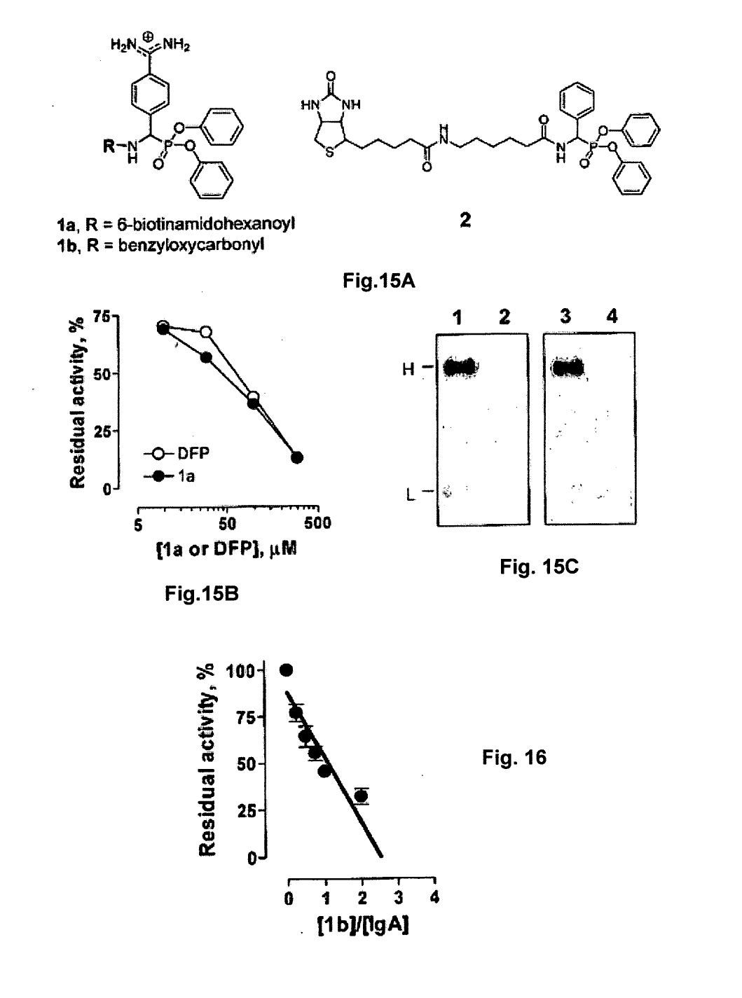

The active site-directed serine protease inhibitors, DFP and diphenyl N-[6-(biotinamido)hexanoyl]amino(4-amidinophenyl)methanephosphonate (1a; FIG. 15A), were used to assess whether IgA-catalyzed Glu-Ala-Arg-AMC proceeds via a serine protease-like mechanism. These compounds were originally developed as covalent inhibitors of conventional serine proteases, and their reactivity with the active sites of IgGs and IgMs has been reported. DFP and 1a inhibited the catalytic activity of serum IgA in a concentration dependent manner (FIG. 15B). Similar results were obtained using IgA isolated from saliva (IC₅₀ values for inhibition by DFP, 50±1 μM; 1a, 37±1 μM). Analysis of 1a-treated IgA samples subjected to heating (100° C., 5 min) and denaturing gel electrophoresis revealed a dominant ˜60 kD 1a-adduct of the heavy chain and a weaker ˜25 kD 1a-adduct of the light chain (FIG. 15C). Treatment with neutral phosphonate 2 under identical conditions failed to yield detectable IgA adducts, as expected from the substrate selectivity studies suggesting the requirement for a positive charge flanking the scissile bond.

The stoichiometry of the reaction was studied by titrating the catalytic activity with limiting amounts of the serine protease inhibitor, diphenyl N-(benzyloxycarbonyl)amino(4-amidinophenyl)methanephosphonate 1b ([1b]/[IgA] ratio, 0.25-2.0) using a monoclonal serum IgA preparation (FIG. 16). The x-intercept of the residual activity (%) versus [1b]/[IgA] plot was 2.5 (r² 0.84), close to the expected stoichiometry of 2 catalytic sites per molecule of IgA.

### Discussion

These studies indicate that IgAs express amidolytic activities superior to the IgG class Abs. Previously, it was reported that an Ab light chain subunit with V region sequence identical to its germline V region counterpart displayed amidolytic and proteolytic activities attributable to a serine protease-like mechanism, suggesting that catalysis is an innate function of the humoral immune system. The catalytic activity is also ubiquitously displayed by IgMs, the first Abs produced in the course of B cell differentiation (54). Previous site-directed mutagenesis and Fab studies have shown that the catalytic site of IgG and IgM Abs is located in the V domains (54). In the present study, the catalytic activities of polyclonal serum IgAs were ˜3-orders of magnitude greater than serum IgGs from the same human donors. Each of the monoclonal IgAs studied displayed the catalytic activity, with the activity levels varying over a 19-fold level, consistent with the expectation of variable activity levels due to differences in the IgA V domains. IgAs of both subclasses (IgA1 and IgA2) displayed the activity, indicating that both molecular forms can support amidolysis. Changes in antigen binding by identical V domains cloned as different IgG isotypes have been described. Study of identical V domains cloned as IgA versus IgG Abs will be necessary in future studies to determine whether the constant domain architecture plays a supportive role in catalysis.

The catalytic activity of the serum IgA was recovered at the precise mass of monomer IgAs (170 kD) from a gel filtration column run in 6M guanidine hydrochloride, a denaturing environment under which noncovalently bound contaminants are removed. The activities of serum and salivary IgA were inhibited virtually completely by the phosphonate diester hapten, a compound originally developed as an irreversible inhibitor of serine proteases, suggesting a serine protease-like mechanism of catalysis. Both types of IgAs formed detectable covalent adducts with the phosphonate diester, consistent with the irreversible mechanism of inhibition. Titration of the activity of a monoclonal IgA using the phosphonate diester inhibitor yielded a value close to the theoretical value of 2 catalytic sites/IgA monomer molecule. If the activity is due to trace protease contamination, the observed stoichiometry will be substantially less than the theoretical value. From these observations, it may be concluded that the innate serine protease-like catalytic activity of Abs is maintained at high levels in the IgA but not the IgG compartment of the expressed Ab repertoire.

The model substrates cleaved by the IgAs are composed of 2-4 amino acids linked via an amide bond to the fluorescent group aminomethylcoumarin. From analysis of the reaction rates for 12 peptide substrates, a pronounced preference was evident for cleavage on the C terminal side of Arg/Lys residues. The basic residue preference of IgAs is similar to that of other classes of Abs described in previous studies (54). IgA from serum and saliva, however, displayed differing levels of preference for various peptide-AMC substrates. For example, Glu-Ala-Arg-AMC was cleaved 59-fold more rapidly than Gly-Gly-Arg-AMC by serum IgA, whereas salivary IgA cleaved these substrates at comparable rates. The catalytic reaction was characterized by high micromolar Kₘ values, suggesting low affinity substrate recognition (Kₘ approximate the inverse equilibrium association constant for noncovalent binding), similar to the properties of previously described IgGs. Importantly, the peptide-AMC substrates are not intended as probes for the adaptive development of noncovalent antigen recognition by IgAs. Rather, these substrates may be viewed as ‘microantigens’ that are accommodated at the catalytic subsite without major engagement of the neighboring Ab subsite responsible for high affinity, noncovalent recognition of the antigen ground state.

This model is supported by the following observation. First, the catalytic rate constants k_{cat} of a proteolytic single chain Fv (tethered VL and VH domains) for the neuropeptide VIP and a peptide-AMC substrate are comparable despite a substantially lower Kₘ for VIP (k_{cat}, turnover number measured at excess substrate concentration). Second, the level of covalent reactivity of a haptenic electrophilic phosphonate (devoid of a peptide epitope) with a panel of human single chain Fv constructs predicted the magnitude of their catalytic activity, suggesting that the nucleophilic site responsible for catalysis does not require the participation of the noncovalent binding subsite (57). Previously, the peptide-AMC substrates have been employed successfully to determine the catalytic potential of monoclonal light chains from multiple myeloma patients, the somatically diversified products of B cells that become cancerous at an advanced differentiation stage (59, 60).

The properties of polyclonal IgAs from healthy humans studied here can be assumed to reflect the immunological selection pressures imposed by a multitude of immunogens, and the adaptive development of individual antigen-specific IgA catalytic activities in response to the selection pressures remains to be examined. Nevinsky and coworkers observed IgA/IgG catalytic potency ratios ranging from ˜0.5-20 for the cleavage of myelin basic protein by IgAs and IgGs purified from the sera from patients with multiple sclerosis (53). A unique method was employed for IgA purification herein, i.e., binding to immobilized Protein A. Protein A is known to bind certain IgAs belonging to the VH3 gene family but not the IgA Fc region, and it is unclear how this property relates to the catalytic activity or whether the observed activity levels are an unbiased representation of the IgA catalytic potential. Also, as the catalysis assays were conducted under limiting concentrations of the substrate, the relative contributions of noncovalent myelin basic protein binding and catalytic turnover are unclear. In comparison, the IgA/IgG activity comparison reported here were obtained at excess concentrations of the peptide-AMC substrate, and the observed rates are a measure of catalytic turnover with minimal contribution of initial noncovalent substrate recognition (under conditions of excess substrate, the reaction proceeds at maximal velocity, independent of Kₘ).

Screening experiments were restricted to a few IgAs, and additional studies are necessary to define the upper limit of the catalytic rate. IgAs are the first line of immune defense against infection in mucosal surfaces and an anti-microbial role for IgA catalytic activities can be hypothesized. Unpublished studies from our group suggest that IgAs present in serum and mucosal secretions catalyze the cleavage of HIV gp120 via recognition of the superantigenic site of this protein (Planque, et al, Innate Superantibodies to HIV gp120. 3rd International AIDS Society Conference on HIV Pathogenesis and Treatment. Jul. 24-27, 2005, Rio de Janeiro, Brazil). Even the promiscuous catalytic activity may help clear unwanted antigens. It was described recently that reduced peptide-AMC cleavage by serum IgG is correlated with death in patients with septic shock. Intravenous infusion of pooled IgG from healthy human donors (IVIG) is employed as a therapy for certain immunodeficiencies, autoimmune disorders and septic shock. Commercially available IVIG preparations showed very low catalytic activity compared to IgAs in the present study, raising the interesting possibility that inclusion of IgAs in IVIG preparations may result in improved efficacy. IgA concentrations in human blood (3.3 mg/ml; ˜20 μM assuming that the IgA is monomeric) are ˜4-5 orders of magnitude greater than conventional enzymes (e.g., thrombin found at ng-μg/ml in serum as a complex with antithrombin III), and IgA k_{cat} values are ˜2-3 orders of magnitude smaller than conventional serine proteases. If catalysis proceeds at the rate observed in the present study, 20 μM IgA will cleave ˜50 mM antigen present at excess concentration (>>Kₘ) over 6 days (corresponding to the approximate half-life of IgA in blood). Maximal velocity conditions can be approached in the case of antigens present at high concentrations, such as bacterial and viral antigens in heavily infected locations.

According to the clonal selection theory, engagement of the B cell receptor (BCR; membrane bound Ig complexed to signal transducing proteins) by the antigen drives cell division and clonal selection. BCR-catalyzed antigen cleavage can be expected to result in release of the antigen fragments, depriving the cells of the proliferative signal. If antigen-BCR binding is the sole selection force, retention and improvement of BCR catalytic activity is possible only to the extent that product release is slower than transmembrane signaling responsible for stimulating cell division. In this case, a possible explanation for the results reported here is that signal transduction by α-class BCRs occurs more rapidly than γ-class BCRs. Another possible explanation is that the BCR catalysis may itself be a selectable activity. Cleavage of covalent bonds by catalysts liberates large amounts of energy compared to far smaller energies released upon noncovalent BCR-antigen engagement. It may be hypothesized that some of the energy can be utilized to induce a productive conformation transition in γ-class BCRs required to induce clonal proliferation.

### Example 2

### Abbreviations

Ab, antibody; AIDS, acquired immune deficiency syndrome; AMC, 7-amino-4-methylcoumarin; BCR, B cell receptor; BSA, bovine serum albumin; CDR, complementary determining region, CHAPS, 3-[(3-cholamidopropyl)dimethylammonio]-1-propanesulfonic acid; sEGFR, soluble epidermal growth factor receptor; FR, framework region; IVIG, intravenous immunoglobulin; HIV, human immunodeficiency virus; LTNP, long term non-progressor; PBMC, peripheral blood mononuclear cells; Rₜ, retention time; RP, rapid progressor; SAg, superantigen; SDS, sodium dodecylsulfate; SFMH study, San Francisco Men's Health Study; SP, slow progressor; V domain, variable domain.

### Anti-HIV Antibody Stimulation in Uninfected Subjects

It is contemplated that anti-HIV antibody synthesis in uninfected subjects is not stimulated by autoantibodies. Rather, the likelihood is that antibody response is to a structurally similar microbial antigen. For example, in the present invention neither anti-Staphylococcal antibodies nor anti-HIV antibodies isolated from infected subjects are not autoantigens. Sequence similarities between HIV gp120, the epitope recognized by humans without HIV infection, and microbial proteins from both Mycobacterium avium (subspecies paratuberculosis K-10, 3-ketosteroid-delta-1-dehydrogenase; GenBank #AAS03800.1) and Streptococcus gordonii (strain Challis substrain, CHI; putative sequence from a probable transcription regulator-like protein lin0474; GenBank #ABV1099.1) are shown.

| | | | | |
|---|---|---|---|---|
| HIV gp120 | 429 | EVGKAMYA | 436 | SEQ ID NO: 6 |
| M. avium | 367 | EVGKAMYA | 374 | SEQ ID NO: 7 |
| HIV gp120 | 421 | KQ-IINMWQEVGK | 432 | SEQ ID NO: 8 |
| S. gordonii | 77 | KQFIINMSQNVGK | 89 | SEQ ID NO: 9 |

This is significant because it influences the choice of the donors and the source fluid used to prepare the therapeutic anti-HIV formulation. For example, saliva is an example of a preferred source of the therapeutic anti-HIV formulation from uninfected humans, as opposed to blood, because commensal microbes, such as S. gordonii, at mucosal surfaces likely stimulate local synthesis of the desired antibodies.

### Proteolytic Antibody Defense Against HIV

The clinical course of HIV-1 infection can be slow, with infected individuals progressing to the symptoms of AIDS at varying rates. Some humans remain free of infection despite repeated exposure to HIV. Certain viral and host factors that influence susceptibility to initial infection and progression of the infection have been identified. These include differences in the infectivity and replication capacity of the infecting virus and mutant viral quasispecies developed subsequently. A well-known host resistance factor is the 32 base pair deletion in the chemokine coreceptor R5 gene, which results in impaired virion entry into host cells. Development of cytotoxic T cells can retard the infection, but escape viral variants eventually emerge. Similarly, adaptive humoral immunity may be protective in the initial stages of infection, but the adaptive response is directed mainly against the highly mutable V3 region of the envelope protein gp120, and Ab-resistant viral quasispecies appear in time.

gp120 contains an antigenic site recognized by Abs present in the preimmune repertoire of humans free of HIV infection. This qualifies gp120 for designation as a B-cell SAg (defined an antigen bound by Abs without the requirement of adaptive sequence diversification of Ab V domains). Synthetic peptide studies suggest that the gp120 SAg site is a conformational epitope composed of peptide determinants 231-260, 331-360 and 421-440 (amino acid numbering according to strain MN sequence. The region composed of residues 421-433 is noteworthy for its high degree of conservation in diverse HIV strains and its role in HIV binding to host cell CD4 receptors. Mutagenesis in this gp120 region and cleavage of the 432-433 peptide bond induces loss of the CD4 binding capability, and contacts between the 421-433 region and CD4 are visible by X-ray crystallography of gp120-soluble CD4 complexes. Encounter with antigens generally stimulates B cell proliferation. SAg binding to the B cell receptor (surface Ig complexed to Igα, Igβ and signal transducing proteins), on the other hand, is thought to induce cellular apoptosis. Currently, there are no reports of adaptively matured Abs that bind the gp120 SAg site in HIV-infected individuals, even as a vigorous adaptive response is mounted to the gp120 immunodominant V3 epitopes.

The possibility of a protective role for Abs that bind the gp120 SAg site is suggested by these observations: (a) Binding of the gp120 SAg site by serum IgG from HIV-seronegative individuals at risk for HIV infection is negatively correlated with the incidence of subsequent HIV infection, and (b) Intravenous infusion of pooled IgG from uninfected monkeys protects recipient monkeys from subsequent challenge with simian immunodeficiency virus, a frequently used model of HIV-1 infection. Mucosal surfaces are the customary route of entry of HIV into the human body. IgAs from the saliva and cervicovaginal lavage fluid of sex workers who remain seronegative despite repeated exposed to HIV are reported to neutralize HIV. Whether the IgAs recognize the gp120 SAg site has not been explored.

The ability of naturally occurring Abs and their subunits to catalyze the cleavage of polypeptide antigens has been documented, e.g., VIP, Arg-vasopression, thyroglobulin, Factor VIII, prothrombin, gp120, gp41, H. pylori urease, casein and myelin basic protein. The proteolytic pathway utilized by the Abs is reminiscent of conventional serine proteases. Site directed mutagenesis and X-ray crystallography of proteolytic Abs have identified activated nucleophilic amino acids similar to those in the catalytic site of enzymes. Moreover, the Abs react irreversibly with electrophilic phosphonates originally developed to react covalently at enzymatic nucleophilic residues.

Promiscuous peptide bond hydrolysis appears to be a heritable and ubiquitous trait of Abs encoded by germline V region genes. IgMs, the first class of Abs produced by B cells, hydrolyze gp120. Adaptively matured proteolytic IgGs synthesized by B cells at their terminal differentiation state, however, are rare, and are encountered primarily in individuals with autoimmune or lymphoproliferative disease. According to the clonal selection theory, BCR-antigen engagement drives cellular proliferation and selection. Rapid BCR-catalyzed antigen hydrolysis and release of antigen fragments may be anticipated to abort the process of clonal selection. Consequently, the development of efficient catalytic Abs over the course of adaptive B cell development is theoretically disfavored unless other immunological factors can play a positive role in this process.

It is demonstrated herein that IgAs from the saliva and serum of humans without HIV infection have the ability to catalyze the cleavage of gp120 efficiently compared to IgGs. The IgAs displayed HIV neutralizing activity in tissue culture, and an electrophilic 421-433 peptide analog blocked the neutralizing activity. The activity of serum IgAs was increased in seropositive subjects with slow progression to AIDS but not rapid progressors. The selective expression of catalytic activity by IgAs appears to be mediated by recognition of the gp120 SAg site and suggests catalytic immunity as a host resistance factor in HIV infection.

### Antibodies

Polyclonal Abs were purified from saliva or serum derived from peripheral venous blood of 4 humans subjects without evidence of HIV infection or immunological disease (1 female and 3 males; age 28-36 years; our laboratory subject codes 2288-2291). Saliva was obtained following chewing of parafilm (61). The Abs were also analyzed as pools of the IgA and IgG fractions purified from 34 humans subjects without HIV infection (17 females, 17 males; age 17-65 years; white 30, black 2, Asian 2; codes 679, 681-689 and 2058-2081). Monoclonal IgAs were purified from sera of patients with multiple myeloma (codes 2573-2587). Abs from 19 HIV-seropositive men enrolled in the SFMH study (62) were purified from two blood samples from each subject (designated bleed 1 and bleed 2; collected between June 1984-January 1990). The patients did not receive anti-retroviral drugs. Bleed 1 was obtained within 6 months of seroconversion. CD4+T cells counts in blood at this time were >325/μl in all subjects. Ten seropositive subjects classified in the SP group, belonged to the top 10 percentile of SFMHS subjects who experienced the least net loss of CD4+ T-cells and had not progressed to AIDS during 78 months of follow-up (age 25-43 years at bleed 1; subject codes 2089-2098). Bleed 2 was obtained from SP subjects 66 months after seroconversion. The second group, designated the rapid progressor (RP) group, consisted of 9 men displaying a decline of CD4+ T cells to <184 μl and development of clinical symptoms of AIDS at the time bleed 2 was obtained (1.5-5 years of seroconversion; age 28-43 years at bleed 1; subject codes, 1930-1938). HIV seroconversion was determined based on the presence of Abs HIV-1 proteins measured by ELISA and confirmed by Western blots. Abs from 10 control men without HIV infection were purified for use as controls (age 27-45; subject codes, 1939-1945, 1953, 1956, 1968). Blood and saliva collection was with informed consent approved by the Univ of Texas Committee for Protection of Human Subjects.

IgA was purified by incubating sera (0.5 ml) with goat anti-human IgA agarose (1 h, 1 ml gel, Sigma-Aldrich) in 50 mM Tris-HCl, pH 7.7, containing 0.1 mM CHAPS, in disposable chromatography columns with rotation, washing the gel washed (4 ml×5) with buffer and elution with 0.1 M glycine, pH 2.7, containing 0.1 mM CHAPS (4 ml) into tubes containing 1 M Tris-HCl, pH 9.0 (0.11 ml). Salivary IgA was purified similarly (7 ml saliva, 0.5 ml anti-IgA settled gel). IgG and IgM fractions were purified on protein G-Sepharose and anti-IgM-agarose columns, respectively, using as starting materials the unbound fractions from the anti-IgA columns (63). Protein concentrations were determined using a microBCA kit (Pierce). Immunoblotting of SDS-electrophoresis gels was with peroxidase-conjugated goat anti-human α, anti-human λ, anti-human κ, and anti-secretory component Ab (Sigma-Aldrich) (63). Gel filtration of serum and salivary IgA previously purified by anti-IgA chromatography (pooled from subject codes 2288-2291) was in 6 M guanidine hydrochloride, pH 6.5, on a Superose-6 FPLC column (0.2 ml/min) as described (64). The nominal mass of eluted protein fractions was determined by comparing Rt values with IgM (900 kD), thyroglobulin monomer (330 kD), IgA (170 kD) and BSA (67 kD). IgA renaturation was by dialysis against 50 mM Tris-HCl, 0.1 M glycine, pH 7.7, containing 0.1 mM CHAPS at 4° C. (Tris-Gly buffer; 2 liters×5, 4 days).

### Proteolysis Assays

Biotin was incorporated at Lys residues in gp120 (MN strain, Protein Science Inc), sEGFR, BSA, HIV Tat (NIH AIDS Res. and Ref. Reagent Prog) and factor VIII C2 fragment (from Dr. K. Pratt) at a stoichiometry of 1-2 mol of biotin/mol protein. Protein hydrolysis was determined by reducing SDS-electrophoresis in duplicate. Following incubation with Abs in 20 μl Tris-Gly buffer containing 67 μg/ml gelatin, the reaction mixtures were boiled in SDS (2%) and 2-mercaptoethanol (3.3%), subjected to electrophoresis and blotting and stained with streptavidin-peroxidase. gp120 cleavage was determined by densitometric measurement of the intact biotinylated gp120 band as [gp120]₀−([gp120]₀×(gp120_{Ab}/gp120_{DIL})), where [gp120]₀, gp120_{Ab}, and gp120_{DIL} represent, respectively, the initial concentration, band intensity in the Ab-containing reaction (in arbitrary volume units, AVU; pixel intensity x band area) and band intensity in reaction mixtures containing diluent. In some studies, the blots were stained with a polyclonal anti-gp120 Ab preparation (65) instead of streptavidin-peroxidase.

In some experiments, the cleavage rate was expressed as the intensity of the 55 kD product band (in AVU, corrected for background intensity observed in reaction mixtures of gp120 incubated in diluent instead of Ab). For cleavage site determination, gp120 was incubated with IgA (pooled from subject codes 2288-2291), the IgA was removed by binding to an anti-human IgA column as described above, and the unbound fraction was lyophilized and redissolved in SDS-electrophoresis buffer containing 2-mercaptoethanol. The gp120 fragments in PVDF blots of SDS-gels were stained with Coomassie blue and subjected to N-terminal sequencing as described previously (33). Inhibitors employed in catalysis studies were: diphenyl N-(6-biotinamidohexanoyl)amino(4-amidinophenyl)methan phosphonate (EP-hapten 1), N-(6-biotinamidohexanoyl)amino(4-amidinophenyl)methanephosphonic acid (non-electrophilic hapten 2), diphenyl N-(benzyloxycarbonyl)amino(4-amidinophenyl)methanephosphonate (EP-hapten 3, corresponding to EP-hapten 1 without biotin), gp120 residues 421-431 (Lys-Gln-Ile-Ile-Asn-Met-Trp-Gln-Glu-Val-Gly) with the amidinophosphonate mimetic of residues 432-433 (Lys-Ala) at the C-terminus (EP-421-433) and VIP containing the amidinophosphonate at Lys20 side chain (EP-VIP). The synthesis and purity of the inhibitors has been described (65). In active site titration studies, monoclonal IgA (from subject code 2582) was incubated with EP-hapten 3 at 37° C. in Tris-Gly buffer containing 0.5% dimethylsulfoxide for 18 h in 96-well plates, the substrate Glu-Ala-Arg-AMC was added and the residual catalytic activity was measured by fluorimetry (λex 360 nm, λem 470 nm) with authentic AMC employed to construct a standard curve (66).

### Phosphonate Binding

Purified IgA (pooled from subjects 2288-2291) was treated with EP hapten 1, control hapten 2, EP-421-433 or EP-VIP and the formation of irreversible adducts was measured by reducing SDS-electrophoresis, electroblotting, staining with a streptavidin-peroxidase conjugate and densitometry (66).

### HIV Neutralization

The studies employed the primary HIV isolate (97ZA009; clade C, R5-dependent), phytohemagglutinin-stimulated peripheral blood mononuclear cells and p24 determinations (67). The IgA or IgG (pooled from subjects 2288-2291; in 10 mM sodium phosphate, 137 mM NaCl, 2.7 mM KCl, pH 7.4) was mixed with an equal volume of HIV [100 TCID50; final volume 0.2 ml RPMI 1640 containing 25% PBS, 0.25% FBS and 3% Natural Human T-Cell Growth Factor(Zeptometrix)]. After incubation for 1 h or 24 h, PBMCs in FBS were (0.05 ml, final concentration 20%) added to and Ab-virus reaction mixtures (68). Some assays were done following IgA treatment with EP-421-433 or EP-VIP (100 μM) followed by determination of the residual HIV neutralizing activity.

### IgA Catalytic Activity.

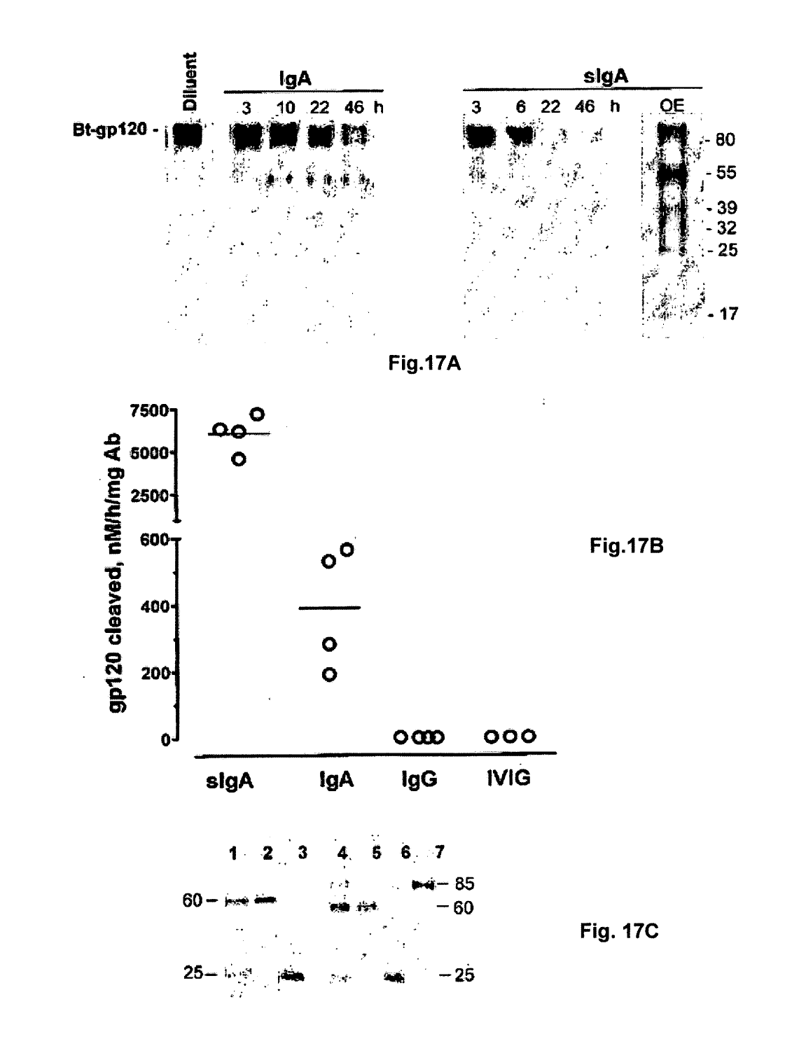

Each IgA preparation purified from the saliva and serum of 4 humans without HIV infection cleaved biotinylated gp120 (Bt-gp120), assessed by depletion of the parent gp120 band and appearance of lower mass fragments in electrophoresis gels (FIG. 17A) (the recombinant protein migrates with nominal mass ˜95 kD, presumably because of incomplete glycosylation in the baculovirus expression system. Biotin detection allows measurement of cleavage rates but does provide accurate information about relative product concentrations, as the Bt-gp120 contains minimal amounts of biotin, ˜1 mol/molgp120, and the products may not necessarily contain the biotin).

The Bt-gp120 product profiles observed using salivary and serum IgA as catalysts were essentially identical (products with nominal mass 80, 55, 39, 32, 25 and 17 kD). The 80 kD band generated in the initial stages of the reaction appeared to be susceptible to further digestion, as the intensity of this band was decreased at the later time points analyzed. The mean proteolytic activity of salivary IgA was 15.4-fold greater than serum IgA. Serum IgG fractions were devoid of detectable activity at the concentrations studied (FIG. 17B). The data in FIG. 17B are expressed per equivalent mass of salivary IgA, serum IgA and serum IgG. As the number of antigen binding sites per unit mass of the Abs are nearly equivalent (1 valency per ˜75-106 kD), the differing activity of various Ab classes can not be due to valency effects. Essentially identical results were obtained using serum IgA and IgG purified from the pooled sera of 34 HIV-seronegative humans (941 nM gp120 cleaved/h/mg IgA; undetectable gp120 cleavage at equivalent IgG concentration; reaction conditions as in FIG. 17A). Several preparations of pooled human IgG (IVIG) are marketed for intravenous infusion for the therapy of immunodeficiency disorders and have also been considered for treatment of HIV infection (69). Like the pooled human IgG prepared in our laboratory, commercial IVIG preparations did not cleave gp120 detectably (Gammagard S/D and Inveegam EN, Baxter; Intratect, Biotech Pharma GmbH).

The electrophoretic homogeneity of the IgG purified as described here has been reported previously. Reducing SDS-electrophoresis of serum IgA obtained by affinity chromatography revealed two protein bands with mass 60 and 25 kD, corresponding to the heavy and light chains subunits (FIGS. 17B-17C). The salivary IgA contained these bands and an additional band stainable with anti-secretory component Ab (85 kD). All of the protein bands detected were also stainable by Abs to the α chain, λ/κ chain or secretory component. The observed IgA subunit bands were not stained by anti-μ or anti-γ Abs, indicating the absence of detectable IgGs or IgMs.

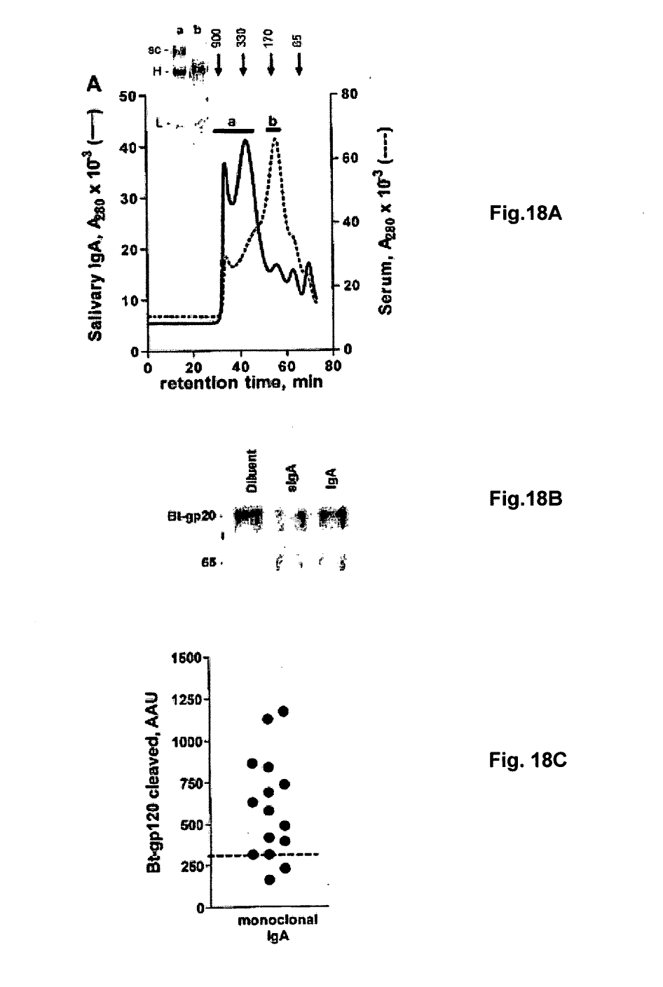

To validate the proteolytic activity, salivary and serum IgA preparations purified by affinity chromatography using the anti-IgA column were subjected to further FPLC-gel filtration in a denaturing solvent (6 M guanidine hydrochloride) (FIG. 18A) as described previously for proteolytic IgGs and IgMs (64). Serum IgA eluted as a major peak at Rₜ 55.2 min with shoulders at 34.0, 44.6, 62.5 min. The nominal mass of the major serum IgA peak at 55.2 min was 153 kD, close to the predicted mass of the secretory component-deficient monomer IgA (170 kD; determined by comparison with the Rₜ of marker proteins). Most of the salivary IgA was recovered in two major peaks at Rₜ 33.7 and 42.7 min, along with minor peaks at Rₜ 55.5, 62.7 and 69.3 min. Nominal mass values for the salivary IgA peaks at Rₜ 33.7 and 42.7 min were, respectively, 915 kD and 433 kD. These observations are consistent with the reported mass heterogeneity of IgAs in blood and mucosal secretions, and the dominance, respectively, of monomer versus polymeric and dimeric IgAs in the former and latter fluids (70). Reducing SDS-electrophoresis profiles of each of the serum and salivary IgA fractions spanning Rₜ 30-57 min indicated subunit profiles that were identical to the affinity purified Ab fractions loaded on the column (FIG. 18B). Ab preparations usually contain small amounts of free Abs subunits and variant oligomeric structures, accounting for the observed minor peaks eluting from the gel filtration column (71).

Following refolding by removal of guanidine hydrochloride, the monomer serum IgA species recovered from the column displayed gp120 cleaving activity (FIG. 18B) that was identical in magnitude to the affinity-purified IgA preparation loaded on the column (respectively, 630 and 823 nM gp120/h/mg IgA). The refolded dimeric and higher order salivary IgA aggregates eluting from the column also displayed gp120 cleaving activity (FIG. 18B), confirming that the predominant form of secreted IgA is catalytically active. Non-IgA proteases in saliva with mass values corresponding to the observed catalytic species (433-915 kD) are not described to our knowledge. The strong denaturant employed for gel filtration is predicted to dissociate and remove any lower mass contaminants that may be bound noncovalently to the affinity-purified IgA loaded on the column. The proteolytic activity of IgA subjected to the denaturing chromatography procedure is inconsistent, therefore, with the presence of non-IgA protease contaminants. The refolded salivary IgA aggregates displayed gp120 cleaving activity that was 4.5 fold lower than the undenatured salivary IgA. A similar denaturant-induced loss of activity due to incomplete refolding into the native protein conformation has been described for other proteolytic antibody preparations.

Further validation studies were conducted using 15 identically-purified monoclonal IgAs from the serum of patients with multiple myeloma (FIG. 18C). Thirteen monoclonal IgAs displayed gp120 cleaving activity and two IgAs were without detectable activity. The electrophoretic profiles of the gp120 reaction products observed using monoclonal IgAs as catalysts were essentially identical to that obtained using polyclonal IgA. Observations that the monoclonal IgAs display differing activity levels are consistent with previously published reports indicating that the Ab variable domains are responsible for cleavage of gp120 and other polypeptide antigens.

Interaction with Electrophilic Phosphonate Hapten

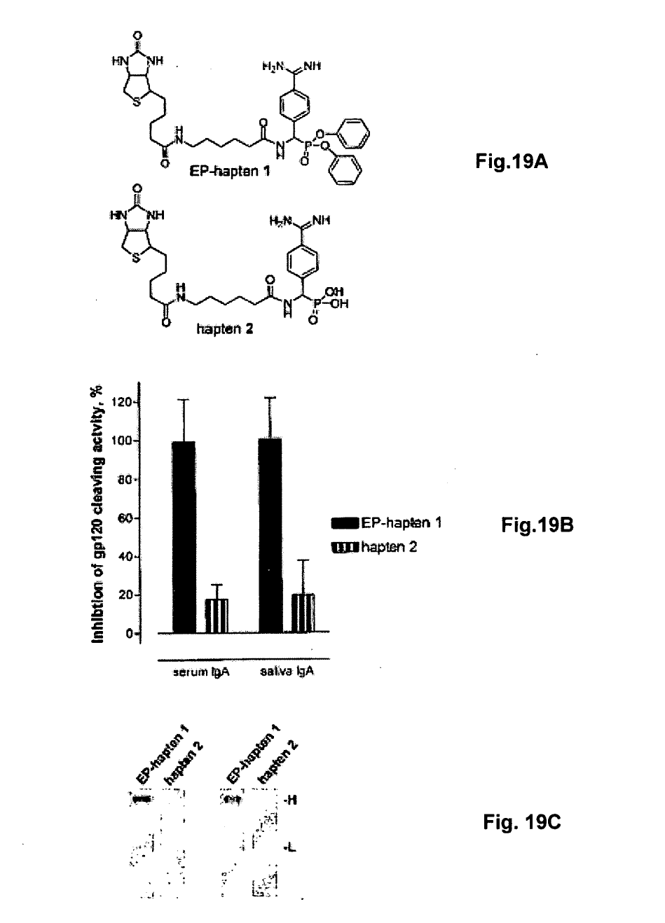

EP-hapten 1 (FIG. 19A) was originally developed as a site directed inhibitor that binds irreversibly to nucleophiles found in the enzymatic active site of serine proteases such as trypsin, and the irreversible reactivity of this compound with catalytic Ab fragments and full-length Abs has also been reported (64, 72). EP-hapten 1 at a concentration of 1 mM markedly inhibited the catalytic activity of salivary and serum IgA (FIG. 19B). Consistent with the predicted covalent mechanism of inhibition, salivary and serum IgA preparations formed adducts with EP-hapten 1 stable to heating (100° C., 5 min) and denaturation with SDS, corresponding to the dominant ˜60 kD heavy chain adduct band and the weaker ˜25 kD light chain adduct band shown in FIG. 19C. With increasing EP hapten 1 concentration, increasing inhibition of gp120 cleavage and formation of adducts with IgA was evident (data not shown). The control hapten 2 is structurally identical to EP-hapten 1 except for the absent phenyl groups at the phosphorus atom, resulting in impaired electrophilic reactivity with enzymatic nucleophiles. Hapten 2 did not inhibit IgA-catalyzed gp120 cleavage or form adducts with the IgAs.

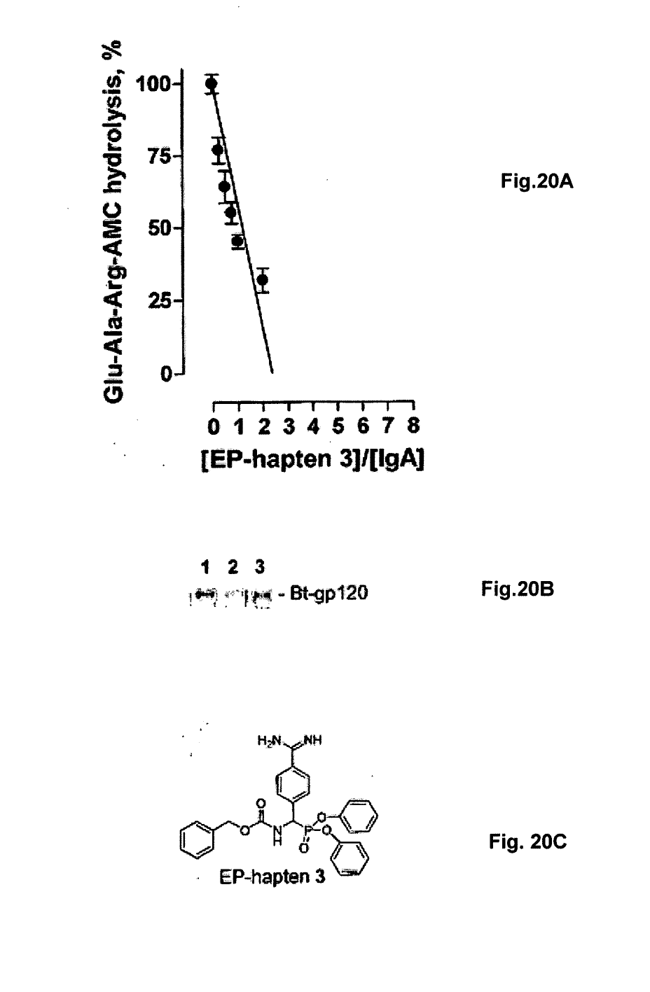

Active site titration studies were conducted using a monoclonal IgA and the serine protease inhibitor EP-hapten 3 (FIG. 20C) and the substrate Glu-Ala-Arg-AMC (FIG. 20A). Fluorimetric measurement of hydrolysis of the Arg-AMC amide bond in this substrate is a convenient method for accurate determination of reaction stoichiometry. The hydrolysis reaction proceeds without the involvement of typical noncovalent interactions accompanying recognition of antigenic epitopes, and similar peptide-AMC substrates have previously been employed as alternate substrates for other catalytic Abs (65, 74). Inclusion of excess Glu-Ala-Arg-AMC in the reaction mixture of gp120 and IgA resulted in complete inhibition of gp120 hydrolysis (FIG. 20B), indicating that the two substrates are cleaved by the same catalytic site. Stoichiometric inhibition of the catalytic activity by EP-hapten 3 was observed, corresponding to complete inhibition of 1 mole IgA by 2.4 moles EP-hapten 3. This value is consistent with the expectation that two EP-hapten 3 molecules should inactivate one IgA molecule (assuming 2 catalytic sites/IgA monomer). If a trace contaminant is responsible for the observed catalytic activity, very small amounts of EP-hapten 3 should suffice to inhibit the activity. Thus, the titration results rule out contaminants as the explanation for catalytic activity.

### Antigen Selectivity and Cleavage Sites

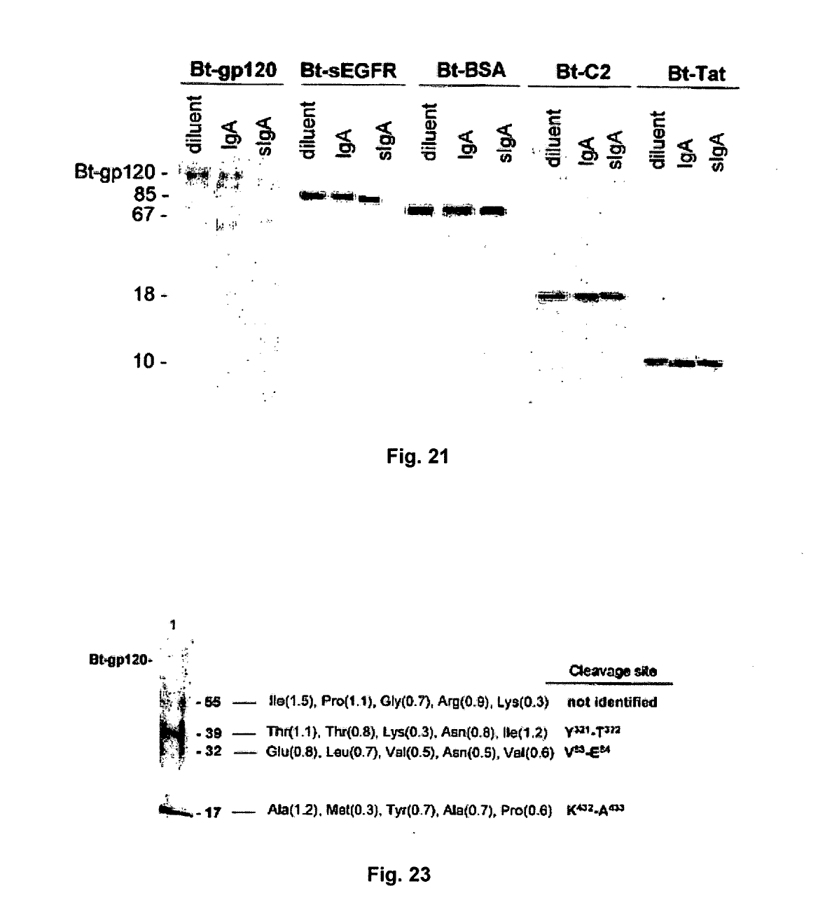

Treatment of Bt-BSA, Bt-FVIII C2 domain, Bt-Tat or Bt-sEGFR with human salivary IgA or serum IgA did not result in noticeable depletion of electrophoresis bands corresponding to the full-length form of these proteins (FIG. 21). Under these conditions, readily detectable Bt-gp120 cleavage was observed.

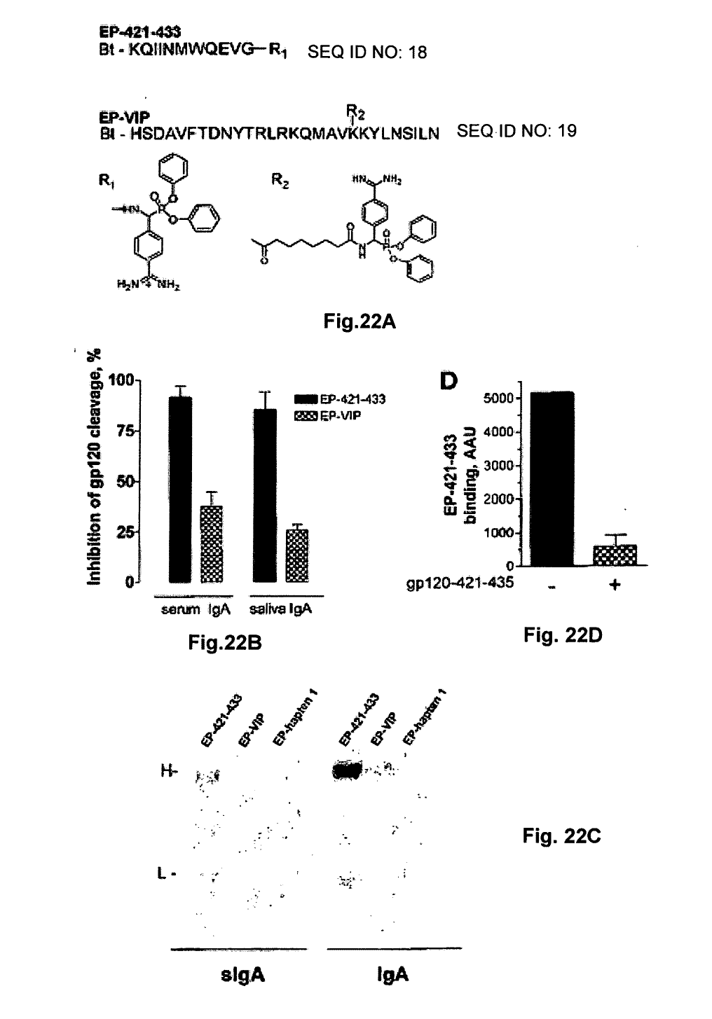

Noncovalent binding of Abs to the gp120 SAg site is inhibited competitively by synthetic peptides containing gp120 residues 421-433 (73). It was reported that the irreversible binding of catalytic IgMs by the electrophilic analog of gp120 residues 421-433 containing the phosphonate diester and biotin groups (EP-421-433; top structure, FIG. 22A). In the present study, inclusion of increasing concentrations of EP-421-433 (10-100 μM) in the reaction mixtures produced a dose-dependent inhibition of the cleavage of Bt-gp120 by salivary IgA (by 21-85%) and serum IgA (by 41-91%). The control probe was EP-VIP (phosphonate containing derivative of VIP, an irrelevant peptide that can inhibit catalysis by reacting covalently with nucleophilic residues but is not anticipated to bind noncovalently to the Abs). Inhibition of IgA catalyzed gp120 cleavage by EP-421-433 was consistently characterized by potency superior to EP-VIP (P=0.01 or smaller, Student's t test; n=4 repeat experiments; FIG. 22B). EP-421-433 also displayed superior irreversible binding to the IgAs compared to control EP-VIP or EP-hapten 1, determined by estimating the biotin content of the protein adduct bands (FIG. 22C). Inclusion of the gp120 peptide 421-436 devoid of the phosphonate group in the reaction mixtures inhibited the formation of the IgA:EP-421-433 adducts (FIG. 22D). These observations suggest a nucleophilic mechanism of IgA catalysis in which noncovalent recognition of SAg peptide region contributes to the observed selectivity for gp120.

To identify the cleavage sites, the digestion of non-biotinylated gp120 by polyclonal salivary IgA was allowed to proceed to near-complete digestion. Following removal of the IgA in the reaction mixture by chromatography on immobilized anti-IgA Abs, the gp120 fragments were subjected to SDS-electrophoresis and N-terminal amino acid sequences (5 cycles). Readily visible product bands at 55, 39 and 17 kD and a faint band at 32 kD were evident (FIG. 23). The 55 kD band yielded a sequence corresponding to the N-terminus of gp120. The remaining bands yielded fragments with N terminal sequences corresponding to gp120 residues 84-88, 322-326 and 433-437, indicating cleavage of the following peptide bonds: Val83-Glu-84 (located in the gp120 C1 domain), Tyr321-Thr322 (V3 domain) and Lys432-433 (C4 domain).

### Neutralizing Activity

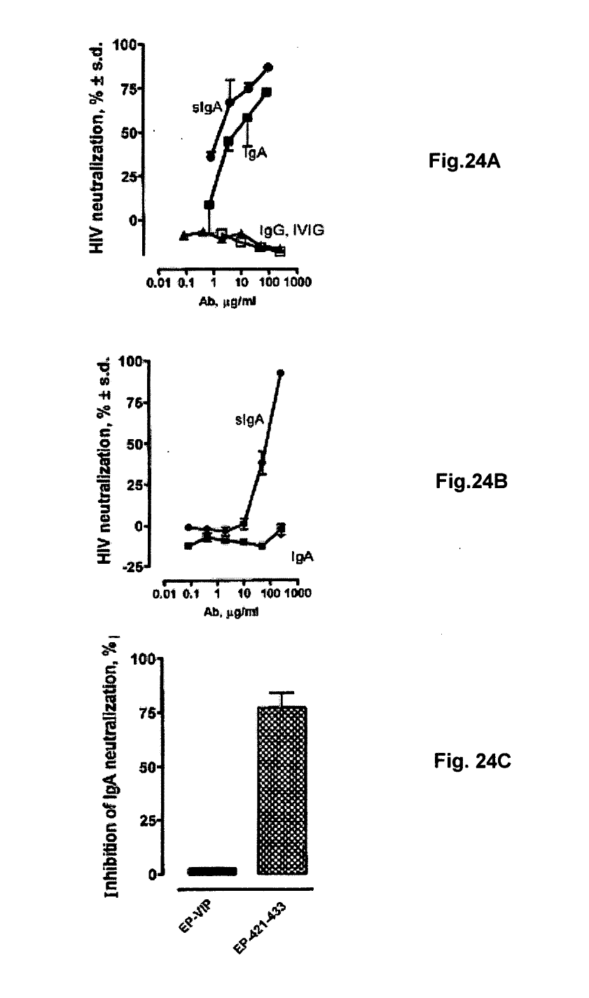

As the initial step in assessment of anti-HIV efficacy, the effect of the Abs on infection of human PBMCs by the HIV-1 strain 97ZA009 (clade C, chemokine coreceptor R5 dependent) was studied. The sequence of gp120 residues 421-433 in this virus strain and the recombinant gp120 employed in catalysis studies is identical except for a conservative Arg/Lys substitution (KQIINMWQEVGR/KA; SEQ ID NO: 10), Los Alamos HIV Sequence Database). Pooled salivary IgA and serum IgA from uninfected donors displayed dose-dependent neutralizing activity. No neutralizing activity was detected in the serum IgG fraction (FIG. 24A). Commercial IVIG preparations containing pooled IgG were also devoid of detectable neutralizing activity (<25% neutralization at 250 μg/ml IVIG). Inclusion of EP-421-433 in the IgA-virus mixture inhibited the neutralizing activity, suggesting that recognition of the 421-433 region is important in the mechanism of neutralization (FIG. 24B). Under the conditions employed in this study, the neutralizing activity of IgA was minimally influenced in the presence of the irrelevant probe EP-VIP. Viral neutralization by salivary IgA was reproducibly observed following comparatively short (1 h) incubation with HIV, whereas neutralization by serum IgA was evident only upon prolonged IgA-virus incubations (24 h; FIG. 24C).

### Catalytic Abs in HIV Infected Subjects

Cleavage of Bt-gp120 by serum IgA from 9 HIV seropositive men with rapid progression (RP) to the clinical symptoms of AIDS, 10 seropositive men with slow progression (SP) to AIDS and 10 uninfected subjects (this is a retrospective study using sera collected in the pre-HAART era) were studied. Saliva from these patients is not available. Secreted IgA may be conceived to impede initial infection across mucosal surfaces. Once HIV gains entry, the activity of systemic antibodies may be the more important variable in progression).

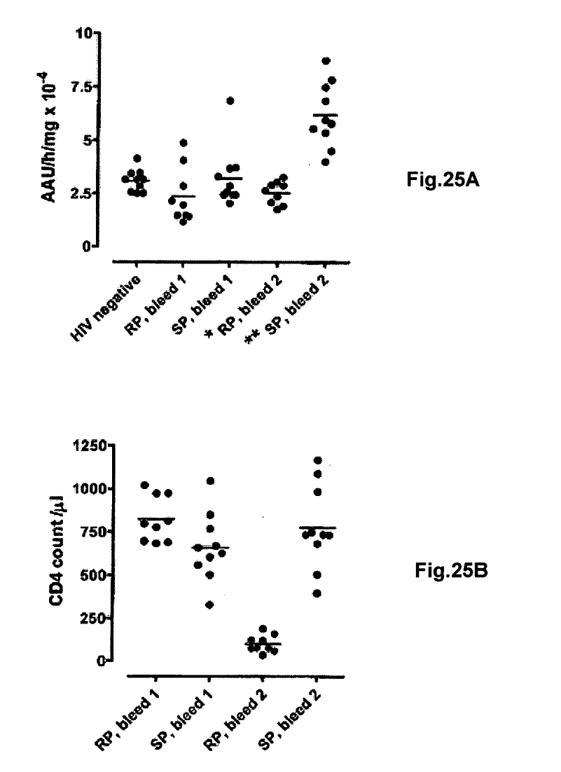

Following seroconversion, the sera were obtained within 6 months (designated bleed 1 in FIG. 25A), 5.5 years (bleed 2 from SP group), or 1-5 years (bleed 2 from RP group). At the time bleed 2 was drawn, the CD4+ T cell counts in the RP group but not the SP group were diminished markedly compared to the normal range (FIG. 25B). The electrophoretic gp120 product profiles observed following digestion by IgAs from the RP and SP groups were essentially identical to the profiles generated by IgAs from uninfected subjects (FIG. 17A). The IgA catalytic activity in the SP group was significantly greater than the RP group or the seronegative group at the bleed 2 stage (P<0.0001, unpaired Mann-Whitney U-test and Student's t-test). A marginal decrease of catalytic activity in the RP group compared to the seronegative group was evident at the bleed 2 stage (P=0.035, Mann-Whitney U-test; P=0.065, Student's t-test). It was reported previously that the cleavage of gp120 by IgMs from HIV-seronegative humans. The gp120 cleaving activities of serum IgMs from the SP group were similar to the RP and uninfected groups (P>0.05; U-test and t-test; data not shown). Cleavage of gp120 by IgAs from two SP group individuals (subject codes 2097 and 2098, bleed 2) was inhibited virtually completely by EP-421-433 (10 μM; % inhibition, 89±6 and 94±12, respectively; reaction conditions as in FIG. 22B). Little or no inhibition of the catalytic reaction was observed at an equivalent concentration of control EP-VIP (<15%).

### Antibody Neutralization of HIV in Long-Term Nonprogressors (LTNP)

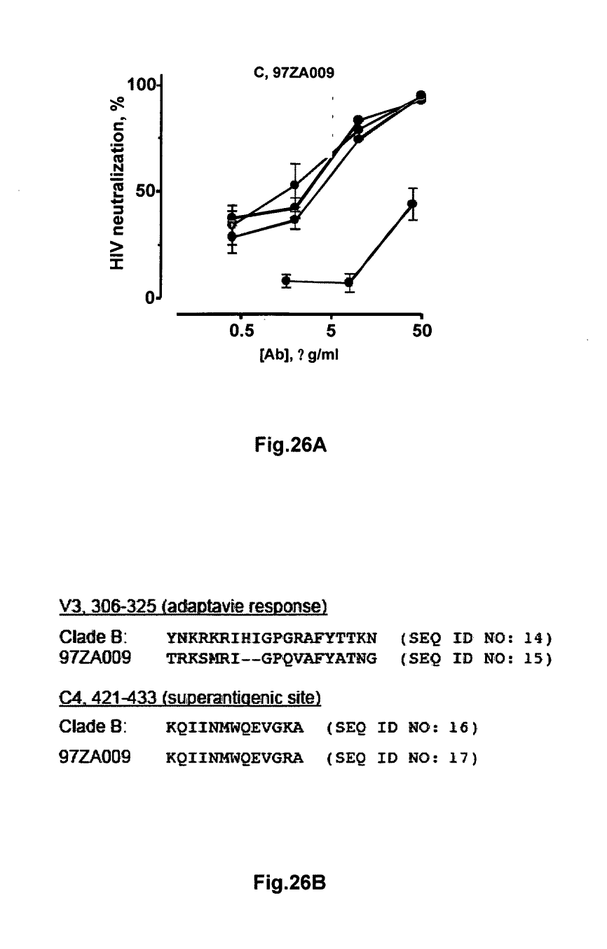

LTNP hemophilia A patients (no progression to AIDS for >18 years, presumptive clade B HIV infection, coinfected with HCV) were studied. Serum IgA preparations from 3 LTNP₁₈ displayed readily detectable gp120 hydrolyzing activity inihibitable by E-421-433. Neutralization assays using purified IgA preparations were performed. Diverse clade B and heterologous clade C strains (1 h incubation with virus; n=11 R5-dependent strains) were neutralized with exceptional potency by the LTNP IgAs (FIG. 26A). The immunodominant V3 306-325 epitope is highly variable in these strains and the 421-433 epitope is mostly conserved (FIG. 26B). In comparison, the reference MAb commonly cited as a broad neutralizing Ig, clone b12, did not neutralize many strains, and when neutralization was evident, the potency was substantially lower. It is contemplated that production of the IgAs is the reason for non-progression.

Table 6 shows heterologous clade C HIV and diverse clade B strain neutralization by IgAs from 3 LTNPs. Clade C strains are 97ZA009, 98TZ013, 98TZ017, Du123, Du156, Du172, and Du422. Clade B strains are ADA, PAVO and QH0692. All strains are R5-dependent. PBMC hosts, p24 assays. Dose-dependent HIV neutralization was evident in all assays.

TABLE 6

| | Number of strains neutralized/ | |
|---|---|---|
| | Number of strains tested | |
| | (IC50 range; mean ± S.D, mg/ml) | |
| | clade B | clade C |
| IgA | 3/3 (0.08-0.20; 0.12 ± 0.07) | 6/6 (0.01-2.70; 0.9 ± 1.3) |
| 2857 | | |
| IgA | 3/3 (0.002-1.000; 0.37 ± 0.55) | 7/7 (0.008-1.960; 0.5 ± 1.0) |
| 2866 | | |
| IgA | 3/3 (0.20-0.80; 0.53 ± 0.33) | 7/7 (0.10-10.70; 2.4 ± 3.8) |
| 2886 | | |
| IgG | 3/4 (0.90-10.00; 3.96 ± 5.22) | 3/8 (4.10-10.50; 6.1 ± 3.5) |
| B12 | | |

### Discussion

Like IgGs, IgAs are produced by differentiated B cells and usually contain V domains with sequences that have been diversified adaptively to varying degree. Unlike IgGs, IgAs from humans without HIV infection catalyzed the cleavage of gp120 potently and selectively. Peptide bond cleavage and noncovalent recognition of the SAg site are thought to be innate, germline V gene encoded functions. Electrophilic phosphonates originally developed as covalent serine protease inhibitors inhibited IgA-catalyzed cleavage of gp120 and were bound irreversibly by the IgAs, suggesting a serine protease-like mechanism of catalysis. The electrophilic analog of residues 421-433, corresponding to a component of the gp120 SAg site, was recognized selectively by the IgAs. One of the scissile peptide bond is located within this gp120 region (residues 432-433). These properties are similar to those of the previously described proteolytic IgMs. There is no requirement, therefore, for de novo generation of the gp120 cleaving activity in the IgAs over the course of B cell maturation, and the activity data suggest that the proteolytic function is retained and improved during V domain sequence diversification and IgA class switching, but not IgG class switching.

Salivary IgA consistently displayed superior catalytic activity compared to serum IgA. IgA in mucosal secretions exists predominantly in dimer and higher order aggregation states and the possibility that the constant domain architecture helps maintain catalytic site integrity is not excluded. Chemical factors that may influence the level of catalysis include the strength of noncovalent gp120 recognition, nucleophilic reactivity of the IgA combining site, and ability to facilitate events in the catalytic cycle after the nucleophilic step is complete, i.e., water attack on the acyl-Ab covalent intermediate and product release. Dissection of the structural basis of gp120 catalysis will require additional studies using monoclonal IgAs with known V domain combining site structures. The crystal structure of a gp120-cleaving IgM has recently been solved and suggests that a Ser-Arg-Glu triad is responsible for the observed nucleophilic and catalytic activities (74).

Polypeptides unrelated to gp120 were not cleaved by the IgAs. EP-421-433 inhibited the cleavage of gp120 and displayed superior irreversible binding to the IgAs compared to the irrelevant EP-VIP probe. Selective gp120 cleavage by the IgAs is attributable, therefore, at least in part to nucleophilic attack on the protein coordinated with noncovalent recognition at the 421-433 region. Three gp120 peptide bonds were cleaved by polyclonal IgA, one of which was located within the gp120 421-433 region (residues 432-433). The regions containing the other two cleavage sites have not been linked previously to the SAg properties of gp120. The gp120 product profiles using monoclonal and polyclonal IgA catalysts were identical, suggesting that a single Ab reactive with the 421-433 region may cleave bonds located outside this region. Studies on cleavage of other polypeptide antigens by monoclonal Abs have also indicated that a single Ab can cleave multiple peptide bonds.

The reaction profile may be understood from the previously-proposed split site model of catalysis (75), in which distinct Ab subsites are responsible for noncovalent binding and catalysis, and the hydrolytic reaction can occur at distant bonds outside the epitope responsible for initial noncovalent antigen-Ab binding. The model proposes formation of alternate ground state complexes containing different peptide bonds positioned in register with the catalytic site. When the Abs recognizes a conformational epitope, the alternate cleavage sites can be distant in the linear sequence but they must be spatially adjacent. Another factor is the likely utilization of the initial cleavage product as a substrate for further digestion. The initial cleavage product may adopt a conformation distinct from the corresponding region of full-length gp120. Such a conformational change, in turn, may enable attack by the Ab at a peptide bond that is inaccessible in the native antigen. Visualization of the initial IgA-catalyzed gp120 cleavage reaction requires the inclusion of the reductant (2-mercaptoehanol) at the SDS-electrophoresis stage, suggesting that the gp120 fragments remain tethered via S—S bridges within a single molecule. As the cleavage reaction releases the molecule from energetic constraints imposed by the intact protein backbone, the cleaved, S—S tethered gp120 can undergo a conformational transition.

Distinct V domain sites are thought to mediate Ab recognition of the SAg site and conventional antigenic epitopes. The two types of interactions are characterized, respectively, by more heavily weighted contributions from the comparatively conserved FRs versus the more diverse CDRs. Recognition of the gp120 SAg site has been attributed to V_{H} domain residues located in FR1 and FR3 along with certain CDR1 residues, whereas Ab recognition of conventional antigenic epitopes is dominated by contacts at the CDRs. One explanation for the existence of the proteolytic IgAs in humans free of HIV infection is that the SAg site recognition capability of the FR-dominated site is coincidentally retained as the CDRs undergo sequence diversification to recognize other, unrelated antigenic epitopes. The FRs are susceptible to limited sequence diversification (albeit at levels lower than the CDRs), and certain CDR residues also provide a limited contribution to SAg binding. The second possibility, therefore, is that SAg site recognition can improve adaptively, potentially driven by an antigenic epitope bearing structural similarity to the gp120 SAg site. Any noteworthy sequence identities between gp120 residues 421-433 and known human proteins were not able to be identified by inspection of the sequence databases. However, 27 of 39 nucleotides encoding these gp120 residues are identical to a human endogenous retroviral sequence (HERV; Paces, J., A. Pavlicek, and V. Paces. 2002. HERVd: database of human endogenous retroviruses. Nucleic Acids Res. 30:205-206); the consensus nucleotide sequence for clade B gp120 residues 421-433 is CCGTATGTAACGAAAAGGATGAAAGACGGTGTACAAATA (SEQ ID NO: 11). The sequence for HERV rv-012650 (family HERVL47, chromosome X, is TTAGATCTGATGAAAAGGATGAAAGAAATTTTTCAAAAA(SEQ ID NO: 12; identities underlined).

No other evidence is available at present linking HERVs and catalytic Abs to gp120, but this point is of substantial interest for future studies. First, to the extent that SAg recognition by Abs has evolved as an innate immune function to defend against microbial infection, a connection between this activity and HERVs could be interpreted to imply the existence of an ancient HIV-related virus. Second, increased HERV expression is a frequent finding in systemic lupus erythematosus and other autoimmune diseases, and this phenomenon may be a factor in unexplained observations of increased Abs to the gp120 peptide 421-436 in patients with lupus. Several clinical case studies have commented on the low frequency of coexistent lupus and HIV infection and a single chain Fv (Ab V_{L} and V_{H} domains linked by a short peptide) isolated from a lupus Fv library displayed the ability to bind the gp120 421-436 region and neutralize the infectivity of primary HIV isolates in tissue culture.

HIV infection is not known or expected to induce Abs directed to the gp120 SAg site. Several reports indicate that Abs containing V_{H} domains of the V_{H}3 family can bind B cells SAgs preferentially. Diminished V_{H}3+ B cell levels and V_{H}3+ immunoglobulin levels have been reported in HIV infected subjects. Other B cell SAgs, i.e., Staphylococcal protein A and Streptococcal protein L, are reported to induce B cell apoptosis. A statistically significant increase of the IgA catalytic activity was evident in the subgroup of HIV seropositive subjects with slow progression to AIDS, whereas the activity was unchanged or marginally reduced in subjects progressing to AIDS. Individuals with slow clinical progression are comparatively rare, and left untreated, most seropositive subjects display reduced CD4+ T cell counts and opportunistic infections characteristic of AIDS. No difference was evident between the gp120 cleavage patterns observed using IgAs from seronegative subjects and slow progressors, and both types of IgAs reacted preferentially with the EP-431-433 probe. This suggests that increased catalytic cleavage in the slow progressor group represents an amplified response to the gp120 SAg site (as opposed to the conventional Ab response to the immunodominant V3 region).

Taken together, these studies suggest the hypothesis that individuals with slow progression to AIDS can mount a beneficial catalytic immune response to the gp120 SAg site. This contrasts with the anticipation that SAg sites are generally unable to induce a specific Ab response. Understanding how the restrictions on anti-SAg site catalytic IgAs can be overcome is of interest for development of novel HIV vaccine candidates. No information is presently available concerning the role of T cells in the production of anti-SAg Abs. Peptides spanning the 421-433 SAg region have been recognized as effective T cell epitopes. The possibility cannot be excluded that enhanced development of T helper cells promotes the production of anti-SAg catalytic IgAs in slow progressor subjects. At the level of the B cells, release of gp120 fragments following BCR catalyzed cleavage at the SAg site of the protein may be predicted to abort the apoptotic signaling pathway induced by gp120-BCR binding, imparting a survival advantage to cells expressing catalytic BCRs. Moreover, there is no assurance that the functional outcomes of proteolysis and noncovalent BCR occupancy are identical. Peptide bond hydrolysis liberates considerably greater amounts of energy (˜Δ70 kcal/mole) compared to noncovalent BCR engagement. If the energy is used productively to induce a BCR conformation transition that triggers cellular proliferation (instead of the apoptotic signal transduction pathway), clonal selection of the catalyst-producing cells should ensue. These considerations suggest synthesis of SAg-reactive catalytic IgAs is immunologically feasible, but the precise circumstances permitting their production in the slow progressor group remain to be elucidated.

Insights to the mechanism of gp120-CD4 binding, the first step in HIV entry into cells, have been drawn from mutagenesis X-ray crystallography studies. The CD4-binding site of gp120 appears to be a discontinuous determinant composed of amino acids located in the 2nd, 3rd and 4th conserved segments, i.e., residues 256, 257, 368-370, 421-427 and 457. In the present study, provided sufficient amounts of salivary IgA and serum IgA from uninfected subjects were present in the cultures, robust neutralization of PBMC infection by a primary HIV strain was evident. The neutralizing activity is consistent with the ability of the IgAs to recognize the 421-433 region implicated in CD4 binding. The electrophilic analog of gp120 residues 421-433 inhibited the neutralization whereas the irrelevant electrophilic peptide did not, suggesting interactions at the 421-433 region as an essential step in IgA mediated viral neutralization.

Selective loss of neutralizing activity in the presence of the EP-421-433 probe is also inconsistent with the alternative possibility that neutralization is caused by recognition of a host cell protein (such as CD4 or chemokine coreceptors). The gp120 421-433 region sequence is largely conserved in diverse HIV strains compared to the immunodominant V3 region [percent conservation of residues gp120 421-433 in 550 HIV strains belonging to various clades available in the Los Alamos Database is: A (54) 93%; B (155), 95%; C (111) 97%; D (20) 96%; F (10), 93%; G (11) 90%; CRF (189) 94% (alphabetical letters are clade designations and numbers in parentheses are numbers of strains). For each strain, the number of identities with the consensus residues in the 421-433 epitope (K-Q-I-I/V-N-M-W-Q-E/R/G-V-G-K/Q/R-A; SEQ ID NO: 13) were counted. % conservation was calculated as 100× (number of identities)/total number of residues in the peptide epitope].

Studies with non-catalytic Abs have noted that variations in the neutralization kinetics can also be expected to impact the anti-HIV efficacy. A single catalyst molecule can be reused in repeated reaction cycles to cleave multiple gp120 molecules (in comparison, a noncatalytic Ab can at most inactivate gp120 stoichiometrically upon establishment of equilibrium, e.g., 2 molecules gp120/molecule bivalent IgG). HIV neutralization by serum IgA in the present study was evident only after prolonged incubations with the virus, whereas salivary IgA reproducibly neutralized the virus despite comparatively short Ab-virus incubations. The more rapid action of salivary IgA is consistent with its greater catalytic activity compared to serum IgA (by ˜15 fold). Human IgG preparations purified in our laboratory and commercial IVIG did not display appreciable gp120 cleaving activity, and the IgG and IVIG preparations were also devoid of neutralizing activity at the concentrations tested.

Commercial IVIG has previously been considered for the therapy of HIV infection. To the extent that the catalytic function enhances anti-viral efficacy, pooled secretory human IgAs can be expected to exert potent anti-HIV effects. It was reported previously that the superior VIP neutralizing potency of a catalytic Ab fragment compared to its catalytically deficient His93:Arg mutant. As the wildtype and mutant Abs bind VIP with equivalent affinity, the superior potency of the former was attributed to the catalytic function. In summary, it is indicated that IgAs from uninfected subjects catalyze the cleavage of HIV gp120, the catalytic activity is increased in subjects with slow progression to AIDS, and the IgAs neutralize the infectivity of a primary HIV strain in tissue culture. These results suggest catalytic IgAs as natural defense mediators against the virus.

### Example 3

### IgM Defense Enzymes Directed to Amyloid β Peptide

Antibodies (Abs) with enzymatic activity (abzymes) represent a potentially powerful defense mechanism against toxic polypeptides. The proteolytic function of an abzyme molecule can inactivate the target antigen permanently, and like conventional enzymes, a single abzyme molecule can cleave thousands of antigen molecules. It was shown that the proteolytic activity of Abs is an inherited function encoded by germline V genes. To the extent that adaptive development of the catalytic function is not proscribed by B cell differentiation processes, the humoral immune system should be capable of producing diverse abzymes specific for individual peptide antigens.

Aggregates of β-amyloid peptides (Aβ peptides) accumulate in the brain with advancing age and are thought to contribute to the pathogenesis of Alzheimer's disease (AD). In addition to the proposed deleterious effect of large Aβ fibrillar aggregates, diffusible oligomers of the peptides are thought to be mediators of neurodegeneration. Naturally occurring Aβ peptide-binding Abs have been identified in the sera of control humans and AD patients. The predicted beneficial function of these Abs is increased clearance of Aβ peptides via uptake of immune complexes by Fc-receptor expressing cells (macrophages and microglia) within the brain or by depletion of Aβ peptides in the blood stream. The action of these Abs within the brain, however, may cause untoward effects resulting from the release of inflammatory mediators or cerebral hemorrhage. Provided herein is evidence that humans synthesize Aβ peptide-reactive proteolytic antibodies that are capable of blocking the formation of the peptide aggregates and dissolving peptide aggregates. These observations indicate that abzymes directed to Aβ peptides may be a natural defense mechanism against AD and that these abzymes may offer a means of immunotherapy for AD.

### Methods

Electrophoretically homogeneous IgM and IgG Abs were purified by affinity chromatography (anti-IgM and Protein G columns) (75). Reaction mixtures of the covalently reactive phosphonate diester with a biotin tag (Bt-Z, diphenyl N-[6-(biotinamido)hexanoyl]amino(4-amidinophenyl)methanephosphonate) and Abs were subjected to SDS-electrophoresis followed by biotin detection to determine adduct formation (75). Catalytic activity was evident as appearance of new A220 peaks on reversed phase HPLC columns of reaction mixtures composed of the Abs and synthetic Aβ1-40. Product generation was quantified from peak areas. Product identity was established by online electrospray ionization mass spectrometry (MS) and MS/MS analysis of individual peptide ions. Cleavage of synthetic Boc-Glu(OBzl)-Ala-Arg-aminomethylcoumarin (AMC) was measured by fluorimetric determination of the liberated AMC (4). Peptide aggregates were visualized by atomic force microscopy (AFM) using microcantilever probes allowing height resolution of 10 nm (76).

### IgM Abzyme Cleavage

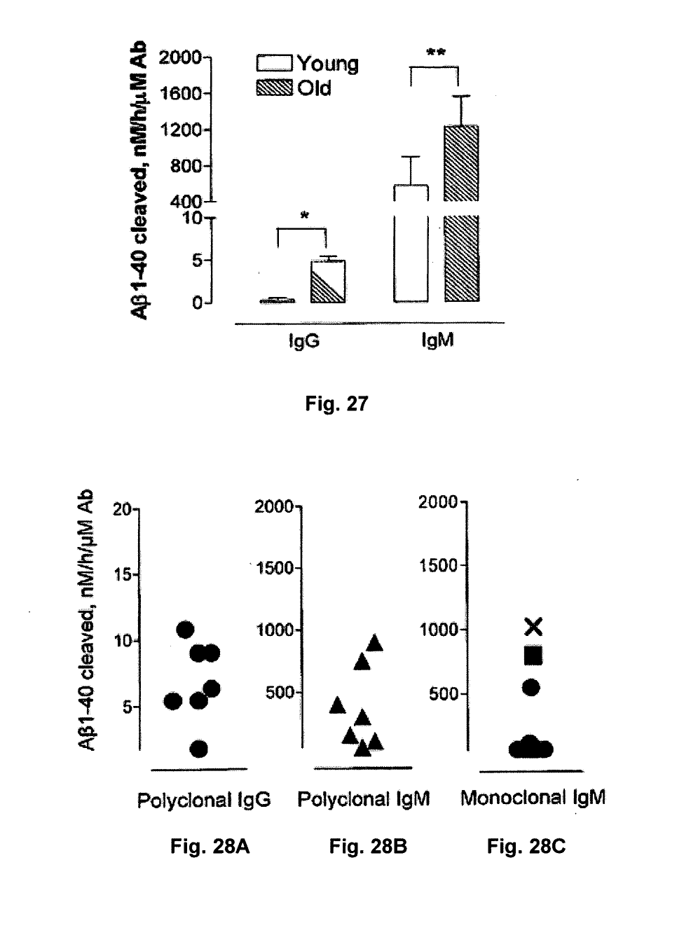

IgM abzymes cleaved Aβ1-40 at rates exceeding IgG Abs (FIG. 27). Like Aβ1-40, Aβ1-42 was also cleaved by the IgM abzymes as determined by HPLC analysis. This is consistent with our belief that proteolysis is an innate immunity function expressed early in the ontogeny of humoral immune responses but subject to deterioration as the responses becomes more specialized for the inciting immunogen. IgM and IgG abzymes from old humans cleaved Aβ1-40 more rapidly than the corresponding Abs from young humans, suggesting that the abzyme response undergoes adaptive maturation as a function of age. This suggests that increasing production of Aβ peptide aggregates with age results in expression of novel conformational epitopes not found in Aβ peptide monomers. Alternatively, persistent exposure of the immune system to the peptide with advancing age may result in a break of immunological tolerance.

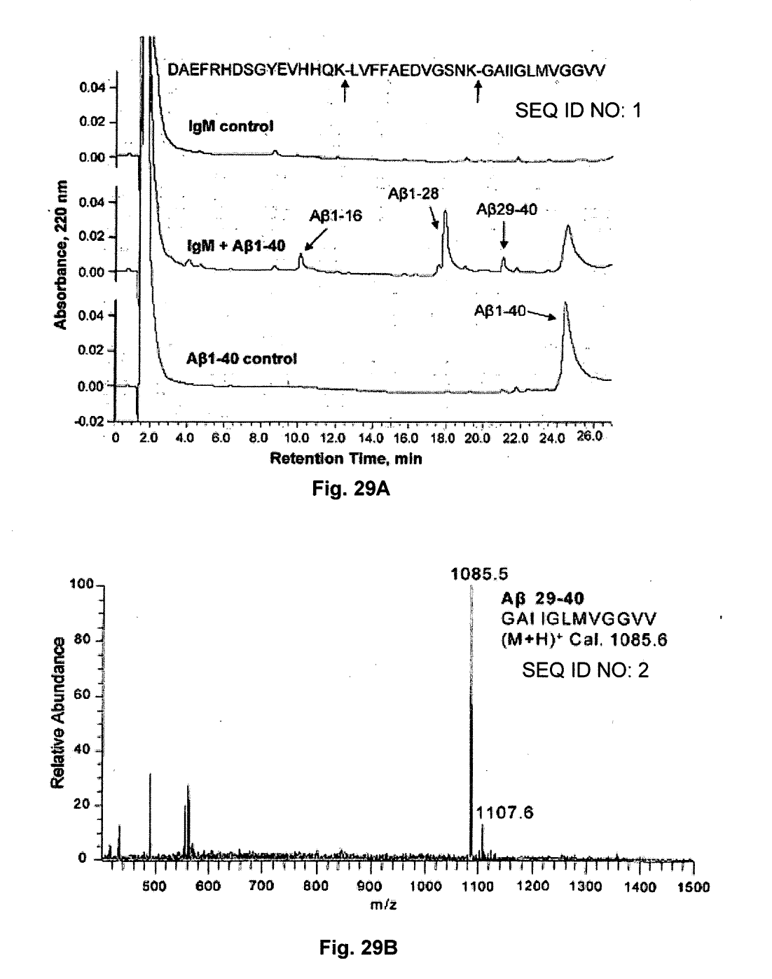

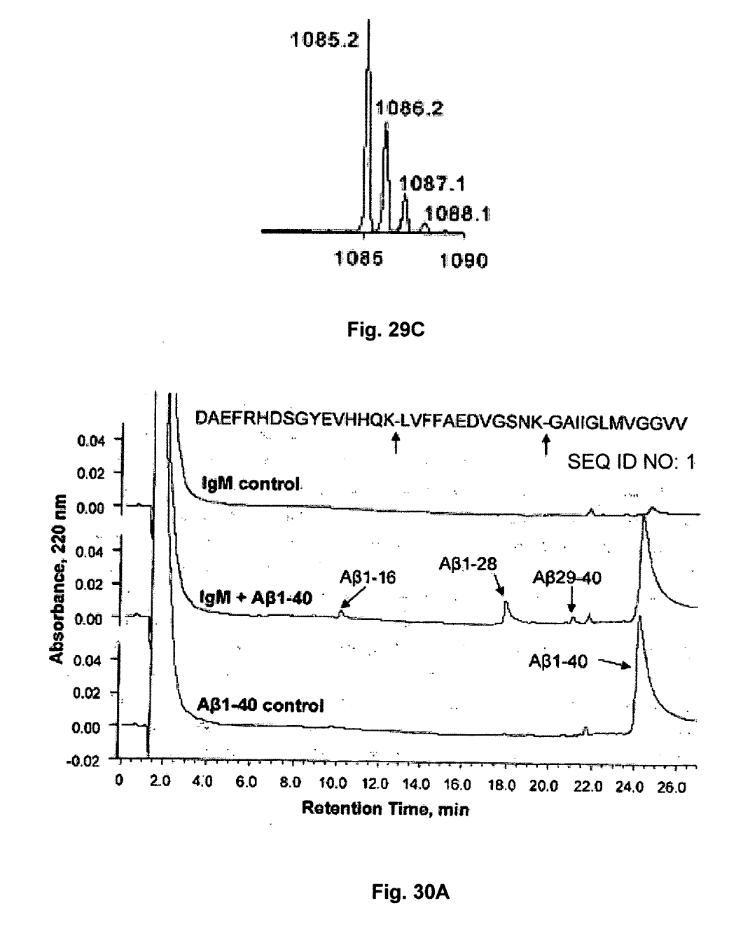

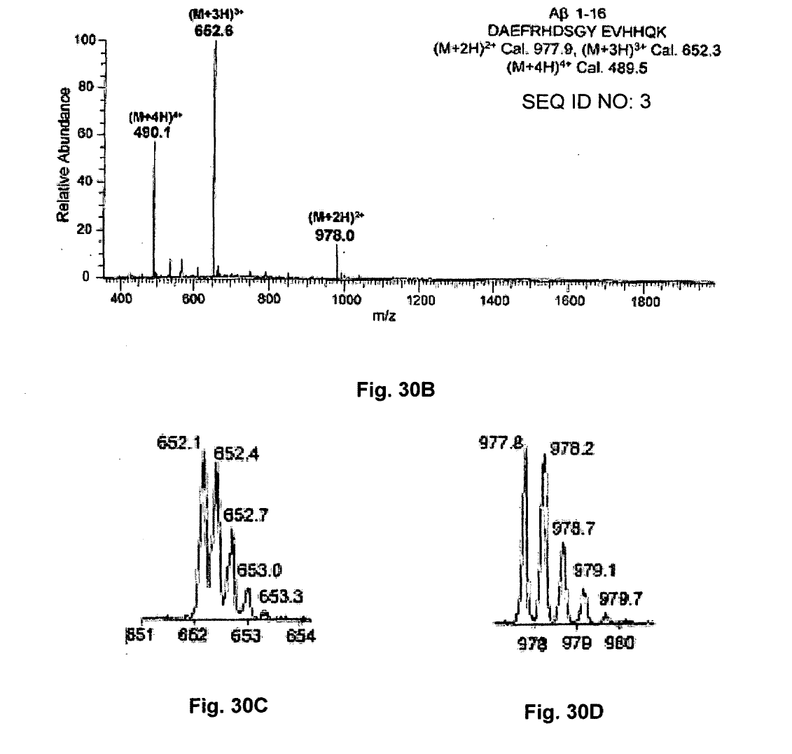

Distinct levels of Aβ1-40 cleavage by identically purified polyclonal and monoclonal Ab preparations were observed, suggesting that the abzyme activity is a polymorphic function associated with the Ab variable domains (FIG. 28). Both polyclonal IgM and a model monoclonal IgM cleaved Aβ1-40 at two bonds, Lys16-Leu17 and Lys28-Gly29. FIG. 29A illustrates the reversed phase HPLC profiles obtained following incubation of Aβ1-40 with monoclonal IgM Yvo. FIG. 29B illustrates the use of electrospray ionization-mass spectroscopy (ESI-mass spectroscopy) to identify the peak at retention time 21.2 min as the Aβ29-40 fragment a zoom scan of spectrum region around m/z peak 1085.5 (FIG. 29C). The observed m/z values in the spectra corresponded exactly to the theoretical m/z for the ions of these fragments, and further, MS/MS analysis of the singly charged species confirmed its identity. FIG. 30 shows the identification of peptide bonds in Aβ1-40 cleaved by polyclonal IgM (pooled from 6 aged subjects). FIG. 30A illustrates the reversed phase HPLC profile of the reaction mixture and FIG. 30B illustrates the identification of the peak at retention time 10.2 min as the Aβ1-16 fragment by ESI-mass spectroscopy including a zoom scan of spectrum region around m/z peaks 652.6 and 978.0 (FIGS. 30C-30D).

The potential of the abzymes to ameliorate the negative effects of Aβ peptides is evident from the kinetic parameters of the monoclonal IgM Yvo. Cleavage of 25-fold greater amounts of Aβ peptide by this abzyme is predicted under the conditions described in Table 6 within one half-life of the anti-Aβ peptide antibodies in blood (3 days) compared to the maximal amount of Aβ peptide bound by comparable non-proteolytic antibodies at equilibrium.

### Apparent Kinetics Parameters for Monoclonal IgM Catalyzed Aβ1-40(3-100 μM) Hydrolysis

IgM Yvo, 200 nM. Data fitted to the Michaelis-Menten-Henri equation (correlation coefficients were 0.95 or better). For illustrative purposes, values for % Aβ cleavage or reversible binding by antibodies with kinetic parameters equivalent to IgM Yvo are included. Under physiological conditions, the Aβ peptide concentrations in blood are ˜0.2 nM, which is <<Km value, and the cleavage rate can be computed as: Pₜ=[Aβ1-40]₀ (1−e^{-k[Ab]·t}), where Pₜ is the product concentration at time t, k is the kinetic efficiency parameter k_{cat}/Km, and [Ab] is the IgM concentration (for this calculation a t value of 56 hours was used, corresponding to the approximate half-life of IgM in blood, and an IgM concentration of 1.1 μM, corresponding approximately to the IgM concentration in blood). At equilibrium, the % of Aβ1-40 existing as immune complexes with a noncatalytic-IgM antibody population that has the same Kd as IgM Yvo (Kd˜Km if k_{cat} is small) can be computed from the equation: [Aβ1-40 complexed to IgM]−([Aβ1-40]₀×[IgM]₀/Kd+[IgM]₀=0. As shown in the Table 7, the binding can never exceed 3.8% of available peptide regardless of the length of incubation, whereas peptide cleavage approaches 90% of the available peptide within one IgM half-life.

TABLE 7
Kinetic parameters

| | |
|---|---|
| Km | 2.8 × 10⁻⁵ M |
| Kcat | 1.8 × 10⁻² min⁻¹ |
| kcat/Km | 6.4 × 10² |
| % A_1β40 cleavage in | 90% |
| 56 h | |
| % Aβ bound at | 3.8% |
| equilibrium | |

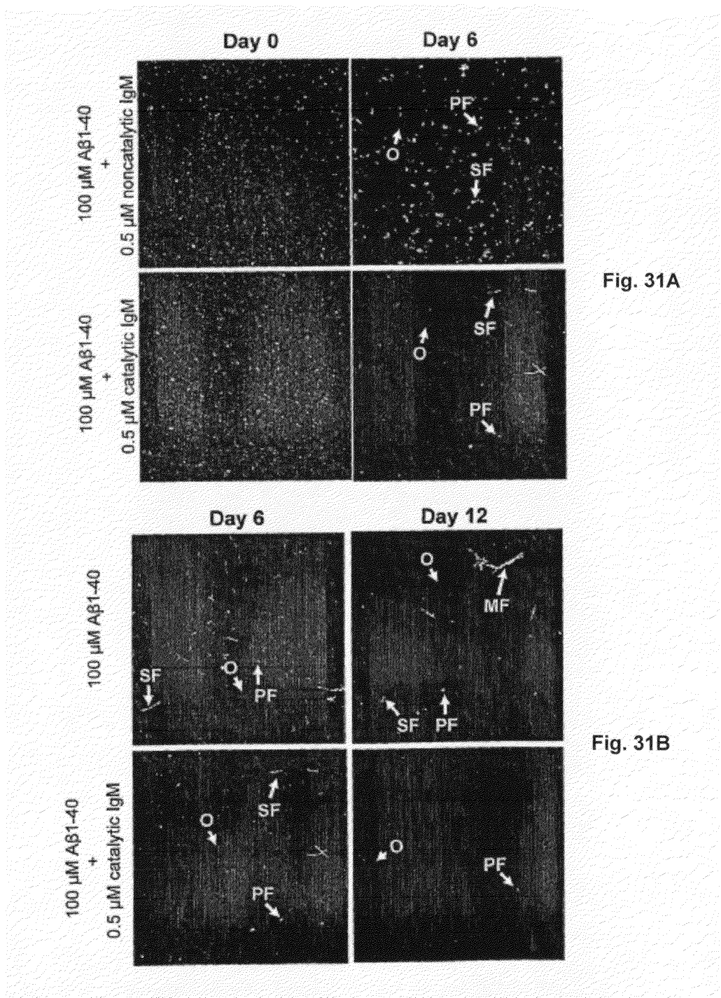

Assembly of Aβ1-40 into fibrillar and oligomer aggregates was blocked by the model monoclonal abzyme. This phenomenon was evident despite a large molar excess of the Aβ peptide over the abzyme (200-fold), consistent with a catalytic mechanism. FIG. 31A illustrates atomic force micrographs of Aβ1-40 treated with the monoclonal IgM for 6 days. By this method, peptide protofibrils, short fibrils and oligomers were visible. Controls included freshly prepared reaction mixtures of the peptide and catalytic IgM as well as the peptide incubated with noncatalytic IgM. FIG. 31B illustrates a decreased Aβ1-40 assemblies in the presence of catalytic IgM Yvo on day 12 compared to day 6. Tables 8 and 9 provide quantitative values for various types of Aβ1-40 assemblies formed in the presence of catalytic IgM Yvo and noncatalytic IgM 1816 on days 6 and 12. The time course studies indicate that the abzyme can also cleave the aggregates, seen evident from disappearance of small amounts of fibrillar and oligomer aggregates observed on day 6 upon further incubation of the mixture. The definitions of oligomers, protofibrils, short fibrils and mature fibrils may be known to the skilled artisan.

Table 8 lists characteristics of Aβ1-40 assemblies formed in the presence of catalytic IgM Yvo and noncatalytic IgM 1816. Data are after incubation for 6 days as in FIG. 30. * P=0.004, ** P=0.002, *** P=0.007. Values are means±SD of 3 analyses. Student's unpaired t test.

TABLE 8

| | IgM Yvo | IgM 1816 |
|---|---|---|
| Number of oligomers (spherical; height, | 123 ± 50.9 | 1301 ± 329* |
| 2-6 nm) | | |
| Number of protofibrils (length, 5-200 nm) | 41.3 ± 26.6 | 810 ± 176** |
| Number of short fibrils (length, 0.2-1 μm) | 4.67 ± 1.15 | 202 ± 66*** |
| Number of mature fibrils (length, >1 μm) | 0.33 ± 0.56 | 0 |
| Length range (fibrils) | 0.3-1.9 μm | 0.2-0.5 μm |
| Height range (fibrils) | 2.3-4.5 nm | 4.3-13.6 nm |

Table 9 lists characteristics of Aβ1-40 assemblies formed after 6 days and 12 days incubation with catalytic IgM Yvo. * P=0.022; ** P=0.013. Values are means±SD of 3 analyses. Student's unpaired t test.

TABLE 9

| | Day 6 | Day 12 |
|---|---|---|
| Number of oligomers (spherical; height, | 123 ± 50.9 | 13 ± 6.1* |
| 2-6 nm) | | |
| Number of protofibrils (length, 5-200 nm) | 41.3 ± 26.6 | 0.7 ± 0.56 |
| Number of short fibrils (length, 0.2-1 μm) | 4.67 ± 1.15 | 0.7 ± 1.15** |
| Number of mature fibrils (length, >1 μm) | 0.33 ± 0.56 | 0 |
| Length range (fibrils) | 0.3-1.9 μm | 0.2-0.3 μm |
| Height range (fibrils) | 2.3-4.5 nm | 4.1-5.9 nm |

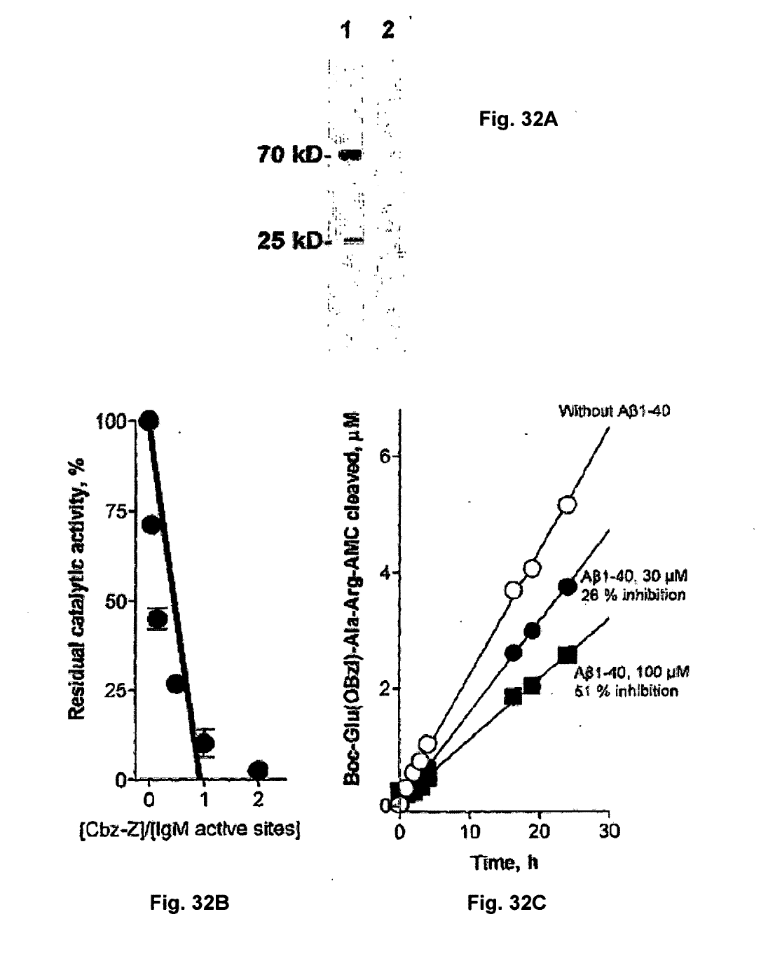

The activity of a model monoclonal IgM was inhibited stoichiometrically by an irreversible phosphonate diester inhibitor of serine proteases and formation of covalent adducts of the Ab with this compound was evident. FIG. 32A shows that IgM Yvo reacts irreversibly with the biotinylated serine protease inhibitor, Bt-Z-2Ph (lane 1) but does not react appreciably with the control probe Bt-Z-2OH under identical conditions (Lane 2). The electrophilicity of the phosphorus atom in the control probe is poor, resulting in its failure to react with enzymatic nucleophiles. The electrophoresis procedure shown in this panel was conducted in the presence of the denaturing reagent SDS and following heating of the reaction mixtures (100° C.), suggesting that observed bands represent covalent adducts, as opposed to noncovalent complexes.

FIG. 32B illustrates the stoichiometric inhibition of IgM Yvo-catalyzed Boc-Glu(OBzl)-Ala-Arg-AMC hydrolysis by the serine protease inhibitor Cbz-Z. The insets illustrate the structures of the substrate and inhibitor. Shown is the plot of residual catalytic activity of the IgM measured as the fluorescence of the aminomethylcoumarin (AMC) leaving group in the presence of varying Cbz-Z concentrations. The value of the x-intercept (about 0.94) was determined from the least-square fit for data points at [Cbz-Z]/[IgM active sites] ratios<2 (1 mole IgM=10 moles IgM active sites). The data suggest that the catalytic activity is attributable to the IgM active sites. FIG. 32C illustrates progress curves for cleavage of Boc-Glu(OBzl)-Ala-Arg-AMC by IgM Yvo in the absence and presence of Aβ1-40 (about 30 and about 100 μM). The observed inhibition suggests that Boc-Glu(OBzl)-Ala-Arg-AMC and Aβ1-40 are cleaved by the same active sites of IgM. These data establish the absence of protease contamination and suggests a nucleophilic mechanism of catalysis akin to previously described proteolytic IgM and IgG abzymes (4).

### Hydrolysis of Aβ40 by IgM Abzyme in Alzheimer's Patients

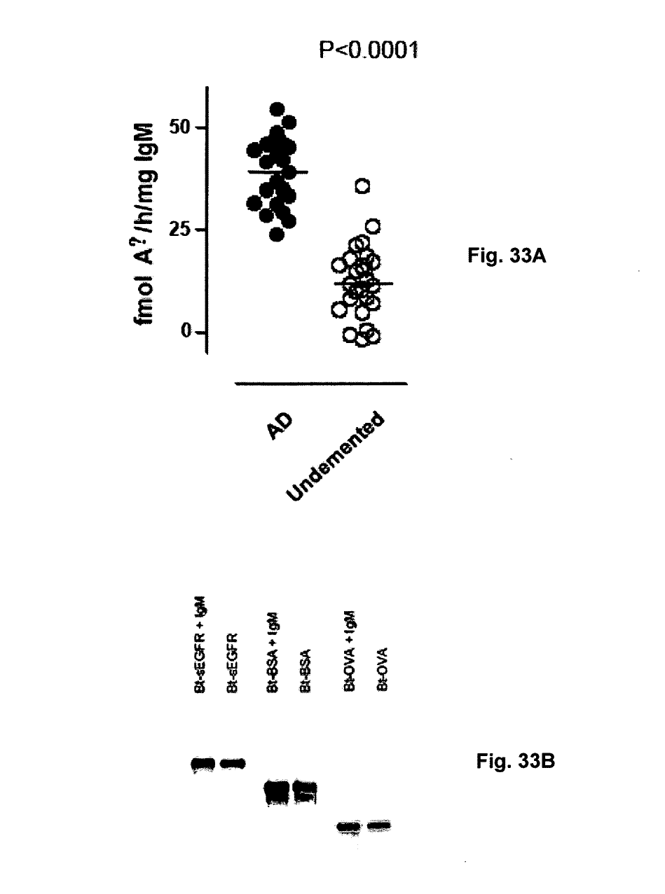

To evaluate disease association, the hydrolysis of ¹²⁵I-Aβ40 by IgM preparations from 25 non-demented elderly individuals (13 females and 12 males, 76±6 years) and 23 elderly AD patients (11 females and 12 males, 76±6 years) (6) was compared. IgMs from the AD group displayed superior hydrolytic activity (P<0.0001; Mann-Whitney U test and Student's t-test; both tests 2 tailed, unpaired; FIG. 33A). There was no gender bias in the distribution of the catalytic activity (P>0.05; non-demented group, 13 females and 12 males; AD group, 11 females and 12 males). The HPLC product profile of Aβ40 treated with polyclonal IgM preparations from 4 AD subjects was essentially identical to the profile using IgM Yvo (FIGS. 29A-29C). A pooled IgM preparation from 5 AD patients with the greatest Aβ40 hydrolyzing activity (codes #2037, 2039, 2041, 2043 and 2044) failed to hydrolyze biotinylated soluble epidermal growth factor receptor, albumin or ovalbumin determined by an electrophoresis assay (FIG. 33B). It may be concluded that increased Aβ40 hydrolysis by IgM preparations from AD patients is not due to an increase of non-specific catalytic activity.

Taken together, these observations indicate that IgM abzymes can exert a protective effect against Aβ peptides in aged humans. The abzymes can potentially clear the peptide without inciting an inflammatory or hemorrhagic response. Thus, in addition to the promise of superior potency due to the catalytic function, abzymes may exert their desired beneficial effect without the toxic complications of stoichiometrically-binding Abs.

### Example 4

### Theory of Catalytic Antibody Occurrence

The following example proposes a theory that helps explains the present invention and clarifies the significance to one skilled in the art.

Catalytic antibodies (Abs) have fascinated several generations of scientists because of their potential to yield insights to protein evolution and routes to novel catalysts on demand, i.e., by inducing adaptive development of specific catalysts to any antigenic substrate. A wealth of empirical information has been gathered, and Abs capable of catalyzing seemingly diverse chemical reactions are documented, including acyl transfers, phosphodiester hydrolyses, phosphorylations, polysaccharide hydrolysis, and water oxidation. Known substrates for catalytic Abs include large antigens (e.g., polypeptides, DNA) and small haptens (e.g., tripeptides, lipids, aldols). Contrary to initial assumptions that Ab catalysis occurs only upon specific recognition of individual substrate structures, Abs can display catalytic activities ranging from the promiscuous (e.g., sequence independent recognition of peptides and aldols with varying substituents neighboring the reaction center) to the highly selective (e.g., cleavage of individual polypeptides enabled by noncovalent recognition of antigenic epitopes).

Catalysts formed by natural immune mechanisms have been identified. The presence of catalytic activities in Abs remains intellectually discomforting because consensus has yet to develop about the biological purpose of the activities. Another source of consternation concerns the relationship between natural and engineered Ab catalysts. Proponents of engineered Abs have argued that as natural Abs usually develop in response to immunological stimulation by antigen ground states, they can not stabilize the transition state, a widely accepted requirement for catalysis. The confusion is due at least in part because no unifying theory of the natural occurrence of catalytic Abs or a rational framework relating the natural and engineered catalytic Abs is available.

Diverse experimental approaches to catalyst identification are described in the literature as follows: (a) Screening for the catalytic activity of spontaneously produced Abs in healthy organisms and individuals with immunological diseases (77); (b) Routine immunizations with ordinary antigens (78); (c) Immunizations with anti-idiotypic Abs raised to the active sites of enzymes (79); and (d) Immunization with stable analogs of unstable reaction intermediates (80).

Each approach has yielded Abs with catalytic activity, but the absent rational foundation has inhibited creative ways of surmounting challenges in the field. A familiar criticism of catalytic Abs is that their turnover (k_{cat}, the first order catalytic rate constant) is lower than of conventional enzymes. For valid comparison of catalytic efficiency, the same molecule must be employed as substrate for Abs and enzymes, as unstable substrates are more rapidly transformed by both classes of catalysts. For energetically undemanding reactions, rate acceleration (k_{cat}/k_{uncat}) is customarily computed to assess the degree to which energy of activation is lowered by the catalyst. Background reaction (k_{uncat}) for demanding reactions such as peptide bond cleavage is very slow [about 7.9×10⁻⁹ min⁻¹], corresponding to a rate acceleration of about >108 for a proteolytic Ab with kcat of about 2.0 min⁻¹.

Only Abs that stabilize the antigen transition state (TS) more than the ground state (GS) can display catalysis, and the turnover is proportional to the difference between the free energy obtained from TS and GS binding (ΔG_{TS}−ΔG_{GS}). Strong antigen_{GS} binding is a historical distinguishing feature of Abs. In comparison, conventional enzymes usually display poor to moderate substrate_{GS} binding. An intrinsic anti-catalytic effect of strong antigen_{GS} binding has been suggested, but this appears to derive more from frustration with empirical findings of slow turnover of catalytic Abs than any theoretical bar to efficient catalysis by Abs. Ab-antigen binding can occur over a large surface area. Achieving a reduction in the reaction activation energy requires the development of TS-specific interactions at groups involved in bond breakage and formation, but there is no reason to believe that the remote interactions established in the ground state complex will be lost as the TS is formed. No anti-catalytic effect is anticipated if the GS binding interactions are preserved in the TS of the Ab-antigen complex.

Antigen_{GS} binding contributes to catalytic efficiency (defined as the k_{cat}/K_{d}) at antigen concentrations below K_{d} (the equilibrium dissociation constant). This situation applies to many protein antigen targets, e.g., trace concentrations of gp120 found in HIV infected subjects. In such examples, even low k_{cat} proteolytic Abs can rapidly degrade the antigen at its biologically relevant concentrations because of strong antigen_{GS} binding. Another functional correlate of strong antigen_{GS} binding is specific catalysis. Indeed, their excellent specificity is a major reason for interest in Abs as catalysts. The importance of this feature can be illustrated using as example the proteolytic activity of Abs. As structurally identical dipeptide units are frequently present in different protein antigens (and within the same antigen), protease specificity for individual protein antigens can not derive from recognition of the scissile peptide bond itself. Contacts formed in the GS remote from the bond breakage/formation steps are vital to effect specific catalysis. Consistent with the seemingly opposing hypotheses about how the catalytic function develops in Abs, understanding the natural selection forces and the best means to engineer catalysts has remained largely conjectural.

Important elements of the theory (but by no means a limitation of this invention) may include: (a) Inherited V domains of Abs contain nucleophilic sites capable of covalent interactions with electrophiles contained in a variety of large and small molecules; (b) The nucleophilic sites are universally expressed in the Abs and are responsible for the promiscuous catalytic activity of Abs produced by the naïve immune system; (c) The nucleophilic reactivity remains coordinated with adaptive development of noncovalent antigen binding activity over the course of B cell maturation. As a result, some adaptively matured Abs can express antigen-specific catalytic activity and improved catalytic efficiency due to decreased K_{d}; (d) Adaptive improvement of catalytic turnover is limited by the rate of B cell receptor signal transduction, as rapid release of antigen fragments from catalytic B cell receptors (BCRs) aborts clonal selection; (e) To the extent that proliferative signals are transmitted at differing rates by BCRs belonging to different Ab classes (μ, δ, α, λ and ε heavy chain classes), the catalytic turnover can develop adaptively in these Ab classes to different extents; (f) Production of catalysts can occur at increased levels under conditions of rapid B cell signaling in autoimmune disease; and (g) Challenge with endogenous electrophilic antigens and electrophilic analogs of peptide bond reaction intermediates induces the adaptive strengthening of Ab nucleophilic reactivity, which can in turn permit more rapid catalysis provided additional structural elements of the catalytic machinery are present.

### Protein Nucleophilic Sites

Nucleophilic catalysis involving formation of covalent reaction intermediates is a major mechanism utilized by enzymes to accelerate chemical reactions, including proteases, esterases, lipases, nucleases, glycosidases and certain synthases. Protein nucleophilicity derives from the precise spatial positioning and intramolecular activation of certain amino acids, e.g., the catalytic triad of serine acylases, in which the Ser oxygen atom is capable of nucleophilic attack on the weakly electrophilic carbon of carbonyl bonds due to the presence of a hydrogen bonding network with His and Asp residues. Until recently, the nucleophiles were thought to be rare end-products of millions of years of protein evolution. Organophosphorus compounds such as difluoroisopropylphosphate and phosphonate diesters contain a strongly electrophilic phosphorus atom, and have been widely employed as covalently reactive probes for enzymatic nucleophiles. It was reported that the V domains of essentially all Abs contain enzyme-like nucleophiles that form covalent adducts with phosphonate diesters containing a positive charge in the immediate vicinity of the phosphorus. Various non-enzymatic, non-Ab proteins also react covalently with the electrophilic phosphorus, and other groups have inferred serine protease-like nucleophiles in peptides and proteins that are not usually classified as enzymes, e.g., glucagon and VIP. Interestingly, certain proteins subjected to irreversible heat denaturation displayed increased nucleophilic reactivity. The nucleophilic sites are undoubtedly formed by spatial proximation and interactions between certain chemical groups in otherwise poorly reactive amino acids, and such interactions are evidently permitted by the non-native folded states of the proteins. It appears, therefore, that nucleophile-electrophile pairing reactions are an intrinsic property of proteins, analogous, for example, to the ability of proteins to engage to varying degrees in hydrogen bonding and electrostatic interactions.

Importantly, the nucleophilic reactivity is a necessary but not sufficient condition for covalent catalysis. For example, catalytic cleavage of peptide bonds by chymotrypsin also requires facilitation of events occurring after formation of the covalent acyl-enzyme intermediate, that is, hydrolysis of the intermediate (deacylation) and release of product peptide fragments from the active site. Abs, while meeting the requirement for nucleophilic reactivity, do not necessarily catalyze proteolytic reactions efficiently.

### Innate, Promiscuous Proteolytic Abs

About 100 VL and VH genes along with smaller numbers of the D and J genes constitute the heritable human repertoire of Abs. The first Abs produced by B lymphocytes over the course of adaptive maturation of the immune response are IgMs. Later, as the V regions diversify by somatic mutation processes, isotype switching occurs, culminating in the production of IgGs, IgAs and IgEs with specific antigen recognition capability. Polyclonal IgMs from immunologically näive mice and healthy humans, and to a lesser extent, the IgGs, display promiscuous nucleophilic and proteolytic activities measured using haptenic phosphonate diesters and small peptide substrates, respectively, limited only by the requirement of a positive charge neighboring the electrophile in these molecules (81). Moreover, μ chain-containing B cell receptors (BCRs) are the dominant nucleophilic proteins expressed on the surface of splenic B cells. Formal proof for the innate origin of the proteolytic activity was obtained from study of the light chain subunit of a proteolytic Ab. The catalytic residues of the light chain identified by site-directed mutagenesis, Ser27a-His93-Asp1, are also present in its germline VL counterpart. Four replacement mutations were identified in the adaptively matured light chain (compared to the germline protein). The matured light chain was reverted to the germline configuration by mutagenesis without loss of catalytic activity, confirming the germline origin of the activity.

Unlike antigen-specific Ab proteases (see below), it appears that promiscuous peptidases are intrinsic components of the immune repertoire. The chemical reactivity of the Abs from healthy individuals probably extends beyond peptide bond hydrolyzing activity, evident from previous observations that human milk contains IgAs with protein kinase activity and that all of randomly picked monoclonal Abs catalyze hydrogen peroxide synthesis. These observations indicate that catalytic activities can arise in Abs by fully natural processes.

### Antigen-Selective Proteolytic Abs

Selective, high affinity recognition of individual antigens is a distinguishing feature of mature Abs. Some antigens, however, are recognized selectively by Abs expressed by Abs encoded by germline Ab V genes, e.g., the identified bacterial proteins Protein A and Protein L and the identified HIV coat protein gp120. These antigens are designated B cell superantigens. Selective recognition of superantigens by preimmune Abs may be rationalized by positing selection of this interaction during the evolution of the V genes, because it resulted in an important survival advantage, i.e., defense against pathogenic microorganisms. The superantigen binding activity is usually mediated by contacts at conserved V domain regions located in the framework regions along with a few contacts at the complementarity determining regions (CDRs). Among several polypeptide substrates analyzed, HIV gp120 was observed to be cleaved selectively by IgMs from uninfected humans. The superantigenic character of gp120 is thought to derive from the recognition of discontinuous peptide segments in the protein, including the segment composed of residues 421-433. Two lines of evidence suggested that the proteolytic IgMs recognize this region of gp120. First, one of the peptide bonds cleaved by the IgMs was located within the superantigenic determinant (Lys432-Ala433). Second, the CRA derivative of the synthetic gp120 peptide corresponding to residues 421-433 formed covalent adducts with the proteolytic Abs at levels exceeding irrelevant peptidyl CRAs and hapten CRAs, suggesting selective noncovalent recognition of the gp120 peptidyl region.

The selectivity of the catalytic IgMs for gp120 can not arise from the local chemical interactions at dipeptide units, as the same dipeptide units are present in other poorly-cleaved proteins. In rare instances, adaptively matured IgGs obtained by experimental immunization can express antigen-selective proteolytic activity attributable to noncovalent recognition of individual epitopes. A role for noncovalent gp120 recognition in the IgM-catalyzed gp120 reaction is supported by the comparatively small Kₘ for the reaction, about 2 orders of magnitude lower than the Kₘ for the promiscuous IgM proteolysis. The noncovalent recognition of the gp120 superantigenic determinant, therefore, appears to facilitate nucleophilic attack on susceptible electrophilic groups by the Abs.

Immunization with the Ground State of Polypeptides

Rapid and specific proteolysis by IgGs elicited by routine polypeptide immunization is an uncommon phenomenon. Immunization with the neuropeptide VIP yielded an IgG with Kₘ in the very low nanomolar range and unconventional kinetics indicating suppression of VIP hydrolysis at elevated IgG concentrations. The isolated light chain subunit of this Ab cleaved VIP according to customary Michaelis-Menten kinetics, albeit with Kₘ substantially greater than the IgG, and the heavy chain subunit was devoid of the activity. The light chain subunits of monoclonal Abs raised by immunization with peptides corresponding to partial sequences of HIV gp41 and CCR5 hydrolyze the corresponding immunogens, but intact IgG did not display the activity.

The case of Bence Jones proteins from multiple myeloma patients, corresponding to the light chain subunits of intact Abs, is relevant, as these proteins could belong to Abs directed to specific foreign or autoantigenic polypeptides (albeit antigens that have not been identified). Frequent proteolysis by panels of light chains isolated from multiple myeloma patients, determined from the ability to cleave model protease substrates has been described (77). The B cells in these patients are thought to become cancerous at an advanced differentiation stage, and the V domains of their Ab products are usually highly mutated. The observed proteolytic activities, however, are promiscuous, and functionally akin to those of germline encoded Abs. Low level promiscuous activities are also detected for the antigen-specific IgGs cited in the preceding paragraph, reflecting the ability of the catalytic sites to accommodate small peptide substrates without making noncovalent contacts typical of high affinity recognition of peptide antigen epitopes.

Another interesting example of antigen-specific proteolysis by IgGs has been described (82). A subpopulation of Hemophilia A patients receiving Factor VII therapy as replacement for the deficient endogenous Factor VII develops IgG class anti-Factor VII Abs, and some IgGs hydrolyze this coagulation promoting protein. Importantly, however, the proteolytic activity may not constitute a routine response to the infused FVIII, as abnormalities in the FVIII gene usually underlie the deficiency of the endogenous protein, and dysfunctional immunological tolerance to FVIII can be conceived to play a role in mounting the unusual catalytic IgG response.

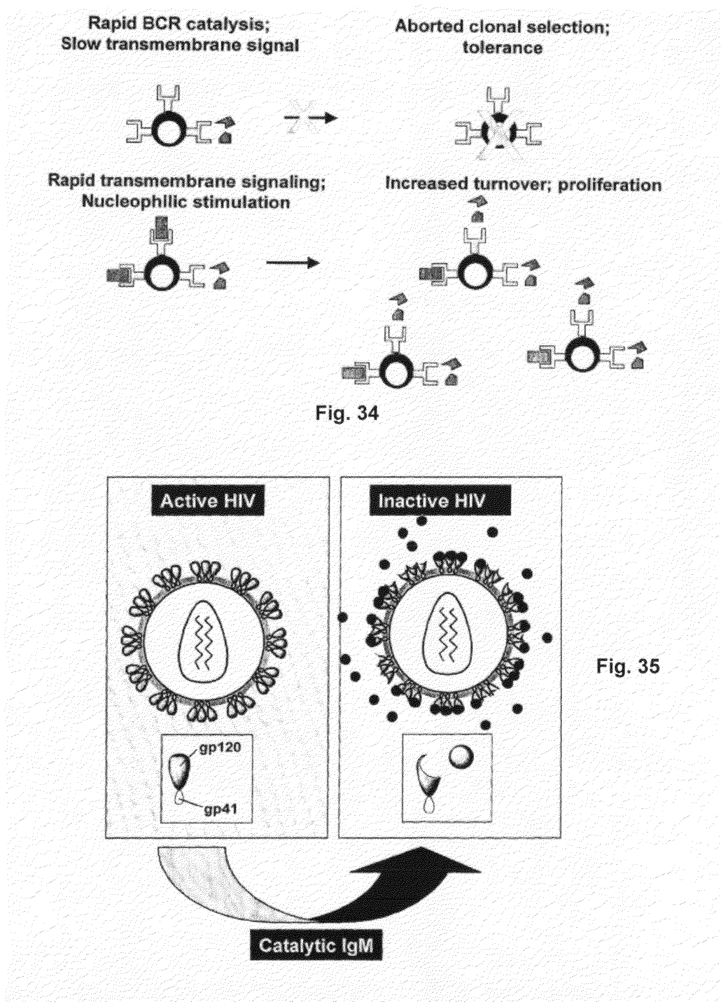

The generation of antigen-specific proteolytic Abs is limited by processes that govern B cell maturation. When the BCR is occupied by the antigen, B cells are driven into the clonal selection pathway. FIG. 34 illustrates the principle that many Ab responses will tend to disfavor improved catalytic turnover, because antigen digestion and release from the B cell receptor (BCR) will induce cessation of cell proliferation. However, there is no hurdle to increased BCR catalytic rates up to the rate of transmembrane BCR signaling. Under certain conditions, further improvements in the rate are feasible, e.g., increased transmembrane signaling rate that is associated with differing classes of BCRs (e.g., μ, α class) or CD19 overexpression, or upon stimulation of the B cells by an endogenous or exogenous electrophilic antigen. Variations can be anticipated in the relative magnitudes of antigen-specific proteolytic activities afforded by adaptively matured IgMs, IgGs and IgAs. This is feasible because BCRs belonging to the μ, γ and α class may induce transmembrane signaling at variable rates depending on the strength of interactions with transducing proteins within the BCRs complex, e.g., CD19, CD22 and Lyn.

### Specific Proteolytic Autoantibodies

The antigen-specific proteolytic activity of Abs was discovered in autoantibody preparations. Patients with several autoimmune diseases are described to be positive for catalytic autoantibodies, suggesting that the restrictions on synthesis of antigen-specific catalysis may be more readily surmounted in autoimmune disease than in the healthy immune system. For instance, VIP-specific catalytic autoantibodies have been observed only in subjects with disease, even though healthy humans also produce VIP-binding Abs (83). The V domains of the proteolytic autoantibodies are adaptively matured, judged from their high affinity for VIP and their extensively mutated complementarity determining regions (which is typical of antigen-specific Abs).

Conditions of accelerated BCR transmembrane signaling could allow B cells to proceed in clonal selection pathways despite increased BCR catalysis. Several reports have linked autoimmunity with dysfunctional B cell signaling due to altered levels of CD19, CD22 and Lyn, proteins contained within the BCR complex. CD19 diminishes the threshold for antigenic stimulation of B cells and CD22 increases the threshold. Lyn, a Src protein tyrosine kinase, is implicated in transduction of antigen-stimulated BCR signaling. Dysfunction of these proteins is associated with increased autoantibody production.

Alternatively, covalent BCR binding by endogenous compounds may induce proliferation of B cells expressing proteolytic BCRs. This is supported by observations that immunization with a model polypeptide CRA stimulates the synthesis of proteolytic Abs. Naturally occurring serine protease inhibitors and reactive carbonyl compounds capable of binding covalently to nucleophiles represent potential endogenous CRAs. For example, a positively charged derivative of pyruvate reacts covalently with the Ser nucleophile of trypsin and thrombin; the positive charge is located at the P1 subsite and does not participate in the covalent reaction]. Additional candidate CRAs are electrophiles produced by lipid peroxidation and protein glycation reactions (Maillard's reaction), processes that occur at enhanced levels in autoimmune disease (84). Examples are 4-hydroxy-2-nonenal and malondialdehyde generated by lipid peroxidation and glyoxal, methylglyoxal and pentosidine generated in sugar metabolism reactions.

### Proteolytic Antibody Engineering

Nucleophilic attack on the carbonyl groups occurs by analogous mechanisms in the course of enzymatic peptide and ester bond cleavage reactions. The hydrolysis small molecule esters by Abs from mice immunized with ester ground state analog and phosphonate monoester transition state analogs (TSAs) has been reported. The esterase activity can be understood from the same principles underlying the proteolytic activity of Abs. The activity was attributed to the ability of the Abs to stabilize the transition state more than the ground state, thereby achieving accelerating the reaction. It was suggested that the TSA immunization induced the de novo adaptive formation of an oxyanion hole in the Abs that stabilized the developing oxyanion in the transition state via noncovalent electrostatic interactions.

The importance of natural immunological mechanisms in producing artificial catalysts is exemplified by the reports describing increased synthesis of esterase Abs in autoimmune mice compared to normal mice in response to TSA immunizations. Another intersection between the fields of natural and engineered catalytic Abs was revealed in studies of reagents originally proposed to serve as noncovalent TSAs. The phosphonate monoester TSAs formed covalent bonds with protein nucleophiles in a manner similar to the electrophilic phosphonate diester probes. This finding explains observations that anti-TSA esterase Abs often use covalent catalytic mechanisms. Certain Abs originally perceived as examples of designer esterases, therefore, appear to owe their catalytic power to innate Ab nucleophilicity. A promising approach to improving the natural nucleophilic activity is immunization with the polypeptide CRAs. Monoclonal IgG clones with specific gp120 cleaving activity have been isolated from mice immunized with the CRA derivative of gp120, and aldolase Abs have been obtained by similar means.

Immunization with Abs to enzyme active sites has been applied to replicate enzyme sites within the Ab combining sites. To the extent that the original enzyme site is selective for a particular substrate, the anti-enzyme idiotypic Abs can be predicted to display a similar selectivity. The induction of proteolytic Abs can be conceived as the field develops and more refined probes capable of capturing Abs that combine the catalytic activity with noncovalent recognition of antigenic epitopes are developed. Structure-guided introduction of a nucleophilic site into Ab V domains by mutagenesis has been reported to impart proteolytic activity to an Ab (85), and CDR mutagenesis followed by phosphonate monoester binding of phage displayed Ab fragments was employed to isolate esterase Abs (86). A covalent phage selection approach has been employed to isolate proteolytic Ab fragments from a lupus phage display library (87).

### Homeostatic Function

Humans inherit about 50 VL and VH gene segments each, and several germline D and J gene segments furnish additional contributions to the diversity of the innate Ab repertoire. To the extent that the proteolysis is an innate function encoded by heritable Ab V domains, it may be predicted that the catalytic activity arose over millions of years of evolution to fulfill some important purpose. High affinity antigen binding characteristic of mammalian Ab responses is usually generated by somatic hypermutation processes acting on the V genes. Ab affinity maturation may occur at limited levels in lower organisms containing the first recognizable immune system. It may be predicted that catalytic immunity is a major defense mechanism against foreign antigens in these organisms.

Consideration of kinetic efficiency of promiscuous peptide cleavage by Abs found in the preimmune repertoire of mice and humans predict that this activity is also important in more evolved immune systems (as opposed to a vestigial function with marginal or no consequences). Apparent turnover numbers (k_{cat}) for our IgM preparations were as high as 2.8 min^{-1 [}12]. Serum IgM concentrations (1.5-2.0 mg/ml; ˜2 μM) are ˜3-4 orders of magnitude greater than conventional enzymes (for example, thrombin found at ng-μg/ml in serum as a complex with antithrombin III), and IgM k_{cat} values are ˜2 orders of magnitude smaller than conventional serine proteases. If catalysis proceeds at the rate observed in vitro, 2 μM human IgM with turnover 2.8/min will cleave ˜24,000 μM peptide substrate present at excess concentration (>>Kₘ) over 3 days (corresponding to the approximate half-life of IgM in blood). Maximal velocity conditions can be approached in the case of antigens present at high concentrations, e.g., albumin and IgG in blood; polypeptides accumulating at locations close to their synthetic site, such as thyroglobulin in the lumen of thyroid follicles; and bacterial and viral antigens in heavily infected locations. A recent study indicated that the promiscuous catalytic activity of IgG from patients who survive septic shock is greater than patients who succumbed, and it was reported previously that diminished promiscuous proteolytic activity in patients with autoimmune disease compared to control non-autoimmune subjects.

Accumulation of amyloid β peptide (Aβ) aggregates in the brain has been proposed as a causal factor in Alzheimer's disease. Monoclonal Abs with Aβ binding activity are reported to clear the peptide aggregates and improve cognition in mouse models of Alzheimer's disease. Aβ1-40 cleavage by polyclonal IgMs and IgGs from young (<35 years) and old humans (>70 years) without evidence of neurodegenerative or autoimmune disease was examined. IgM and IgG preparations from old humans cleaved Aβ1-40, with the IgM displaying 183-fold greater activity than the IgG. The IgMs from young humans cleaved Aβ1-40 at lower levels, and the activity was not detected at all in IgGs from the young humans. Incubation of micromolar Aβ1-40 concentrations with nanomolar concentrations of the monoclonal IgM blocked the formation of peptide fibrils. These indicate suggest that autoantibodies that cleave Aβ1-40 improve adaptively as a function of age and can fulfill a protective function.

IgG Abs that bind the gp120 superantigenic site noncovalently have previously been suggested as resistance factors to the infection. Trimeric gp120 expressed on the HIV surface of is responsible for binding to host cell CD4 receptors as the first step in the infection cycle. Cleavage of gp120 by IgAs and IgMs occurs within a region thought to be important in host cell CD4 binding; the reaction rates suggest that the proteolytic Abs are capable of rapidly neutralizing HIV-1 compared to reversibly binding Abs devoid of proteolytic activity; and the Abs neutralize HIV-1 infection of cultured peripheral blood mononuclear cells. The characteristics of HIV-1 gp120 cleavage by IgMs from uninfected humans indicate that proteolytic Abs constitutes an innate defense system against HIV infection that are capable of imparting resistance or slowing the progression of infection (FIG. 35).

### CRA Inactivation of Pathogenic Antibodies

Autoimmune disease is associated with increased proteolytic autoantibody synthesis. Depletion of VIP and the coagulation Factor VIII by catalytic Abs have been suggested as contributory factors in autoimmune disease and Hemophilia A, respectively. CRAs inactivate proteolytic Abs irreversibly, and inclusion of the appropriate antigenic epitope within the CRA structure is predicted to render the covalent reaction specific for the undesirable Ab subpopulation. This strategy is applicable to permanent inactivation of any pathogenic Ab population regardless of proteolytic activity, as all Abs studied thus far contain a nucleophile within their antigen combining sites that binds covalently to the electrophilic phosphorus of CRAs. Moreover, specific targeting of B cells by the CRAs is conceivable, as the BCR nucleophiles are expressed early in the ontogeny of the Ab response. Because of the irreversible reactivity, the CRAs are predicted to saturate BCRs more readily compared to conventional antigens. BCR saturation is thought to tolerize B cells and the CRAs offer a potential route to induction of antigen-specific tolerance.

### Potential for Clinically Useful Abs

Monoclonal Abs account for a significant proportion of marketed biotechnology products and polyclonal IVIG preparations are useful therapeutic reagents in several diseases. Proteolytic Abs to HIV coat proteins and Aβ peptides are already in hand and HIV infection and Alzheimer's disease are obvious targets for such Abs.

The following references are cited herein.
1. Shuster et al. Science 1992 May 1; 256(5057):665-7.
2. Li et al. J Immunol 1995 Apr. 1, 154(7):3328-32.
3. Bangale et al. FASEB J 2003 Apr. 17, (6):628-35.
4. Saveliev et al. Immunol Left 2003 May 1, 86(3):291-7.
5. Sun et al. J Immunol. 1994 Dec. 1, 153(11):5121-6.
6. Gao et al. J Mol Biol. 1995 Nov. 10, 253(5):658-64.
7. Gololobov et al. Mol Immunol. 1999 December, 36(18):1215-22.
8. Nevinsky et al. Appl Biochem Biotechnol. 1998 October, 75(1):77-91.
9. Kohen et al. FEBS Lett. 1980 Mar. 10, 111 (2):427-31.
10. Kalaga et al. J Immunol 1995 Sep. 1, 155(5):2695-702.
11. Planque et al. J Biol Chem 2004 Apr. 2, 279(14):14024-32.
12. Paul et al. Springer Semin Immunopathol. 2005 March, 26(4):485-503.
13. Lacroix-Desmazes et al. Proc Natl Acad Sci USA. 2005 Mar. 15, 102(11):4109-13.
14. Xu et al. Bioorg Med Chem. 2004 Oct. 15, 12(20):5247-68.
15. Paul et al. J Biol Chem. 2003 May 30, 278(22):20429-35.
16. Mascola et al. J Virol. 1997 October, 71(10):7198-206.
17. Bertolini et al. PCT Int. Appl. WO9805686. 1998 Feb. 12.
18. Jablonowski et al. Clin Investig. 1994 February, 2(3):2204.
19. Stiehm et al. J Infect Dis. 2000 February, 181(2):548-54.
20. Paul et al. J Biol Chem. 2004 Sep. 17, 79(38):39611-9.
21. Rodman et al. J Exp Med 1992 May 1, 175(5):1247-53.
22. Berberian et al. Science. 1993 Sep. 17, 261(5128):1588-91.
23. Karray et al. Proc Natl Acad Sci USA. 1997 Feb. 18, 94(4):1356-60.
24. Lenert et al. Hum Immunol. 1996 August, 49(1):3848.
25. Weksler et al. Exp Gerontol. 2002 July, 37(7):943-8.
26. Paul et al. J Neuroimmunol. 1989 July, 23(2):13342.
27. Bangale et al. Peptides. 2002 December, 23(12):2251-7.
28. Dodel et al. Ann Neurol. 2002 August, 52(2):253-6.
29. Kido et al. J Biol Chem. 1993 Jun. 25, 268(18):13406-13.
30. Kieber-Emmons et al. Biochim Biophys Acta. 1989 Dec. 27, 989(3):281-300.
31. Capon D J and Ward R H. Annu Rev Immunol. 1991, 9:649-78.
32. Gelderblom et al. Lancet. 1985 Nov. 2, 2(8462):1016-7.
33. Brenneman et al. Nature. 1988 Oct. 13, 335(6191):63942.
34. Muller et al. Eur J Pharmacol. 1992 Jul. 1, 226(3):209-14.
35. Hober et al. FEMS Immunol Med Microbiol. 1995 January, 10(2):83-91.
36. Laurent-Crawford et al. Res Virol. 1995 January-February, 146(1):5-17.
37. Stoiber et al. AIDS. 1995 January, 9(1):19-26.
38. Thali et al. J Virol. 1991 November, 65(11):6188-93.
39. Thali et al. J Virol. 1992 September, 66(9):5635-41.
40. Karray et al. Proc Natl Acad Sci USA 1997, 94:1356-1360.
41. Goodglick et al. J Immunol 1995; 155:5151-5159.
42. Bertolini et al. PCT Int. Appl. WO9805686. 1998 Feb. 12.
43. Loomes et al. J Immunol Methods. 1991 Aug. 9, 41(2):209-18.
44. Kabir S. Immunol Methods. 1998 Mar. 15, 212(2):193-211.
45. Planque et al., J Biol Chem 2004 Apr. 2, 279(14):14024-32.
46. Paul et al. J Biol Chem 2004 Sep. 17, 279(38):39611-9.
47. Silverman et al. J Immunol. 1993 Nov. 15, 151(10):5840-55.
48. Pyne et al. Rheumatology (Oxford). 2002 April, 41(4):367-74.
49. Planque et al. J Biol Chem 2003 May 30, 278(22):20436-20443.
50. Cohen et al. Organophosphorus Compounds. In Methods Enzymol. Vol. 11, Enzyme Structure. Hirs CHW. Ed, pp 686-705, Academic Press, New York, 1967.
51. Sun et al. J Immunol 1994 Dec. 1, 153(11):5121-6.
52. Paul et al. Immunol. Lett. 103, 8-16.
53. Polosukhina et al. Med. Sci. Monit. 11, BR266-BR272.
54. Planque et al. J. Biol. Chem. 279, 14024-14032.
55. Nishiyama et al. J. Biol. Chem. 279, 7877-7883.
56. Nishiyama et al. Arch. Biochem. Biophys. 402, 281-288.
57. Planque et al. J. Biol. Chem. 278, 20436-20443.
58. Paul et al. J. Biol. Chem. 276, 28314-28320.
59. Matsuura et al. Biochem. Biophys. Res. Commun. 204, 57-62.
60. Paul et al. J. Biol. Chem. 270, 15257-15261.
61. Krieger et al. (2004) J Appl Physiol. 97, 585-591.
62 Sheppard et al. (1993) AIDS. 7, 1159-1166.
63. Paul et al. (2004) J Biol Chem. 279, 39611-39619.
64. Planque et al. (2004). J Biol Chem. 279, 14024-14032.
65. Paul et al. (2003) J Biol Chem. 278, 20429-20435.
66. Planque et al. (2003) J Biol Chem. 278, 20436-20443.
67. Karle et al. (2004) AIDS. 18, 329-331.
68. Donners et al. (2003)Vaccine. 22, 104-111.
69. Olopoenia et al. (1997) J Natl Med Assoc. 89, 543-547.
70. Brandtzaeg et al. (1999) Immunol Rev. 171, 45-87.
71. Li et al. (2000). Clin Exp Immunol. 12, 261-266.
72. Paul et al. (2001) J Biol Chem. 276, 28314-28320.
73. Goodglick et al. (1995) J Immunol. 155, 5151-5159.
74. Ramsland et al. (2006) Biochem J. 395, 473-481.
75. Paul, S. (1996) Mol Biotechnol. 5, 197-207.
75. Planque et al. J Biol Chem. 2004 Apr. 2, 279(14):14024-32.
76. Stine et al. J Biol Chem. 2003 Mar. 28, 278(13):11612-22.
77. Matsuura et al. Biochem Biophys Res Commun 1994, 204:57-62.
78 Paul et al. J Biol Chem 1992, 267:13142-13145.
79. Izadyar et al. Proc Natl Acad Sci USA. 1993, 90:8876-8880.
80. Tramontano et al. Science 1986, 234:1566-1570.
81. Planque et al. J Biol Chem 2003, 278:20436-20443.
82. Lacroix-Desmazes et al. Nat Med 1999; 5:1044-1047.
83. Bangale et al. Peptides 2002, 23:2251-2257.
84. Ames et al. Rheumatol 1999, 38:529-534.
85. Chen et al. Am J Hematol 1993, 44:276-279.
86. Lacroix-Desmazes et al. Proc Natl Acad Sci USA 2005; 102:4109-4113.
87. Krebs et al. Biochemistry 1995, 34:720-723.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages inherent herein. The present examples, along with the methods, procedures, systems, and/or applications described herein are presently representative of preferred embodiments, are exemplary and are not intended as limitations on the scope of the invention. Changes therein and other uses will occur to those skilled in the art which are encompassed within the spirit of the invention as defined by the scope of the claims.

What is claimed is:

1. An isolated and purified pooled immunoglobulin preparation comprising pooled immunoglobulins of defined class having catalytic activity.

2. The pooled catalytic immunoglobulins of claim 1, wherein the immunoglobulins are also defined by subclass.

3. The pooled catalytic immunoglobulins of claim 1, wherein the immunoglobulins are isolated from four, ten, twenty, thirty, thirty-five, fifty, one-hundred or more humans.

4. The pooled catalytic immunoglobulins of claim 1, wherein the immunoglobulins are isolated from a mucosal secretion, including but not limited to saliva and milk.

5. The pooled catalytic immunoglobulins of claim 1, wherein the immunoglobulins are isolated from blood.

6. The pooled catalytic immunoglobulins of claim 1, wherein the class of the immunoglobulins is IgA, IgM, IgG or a mixtures or combination thereof.

7. The pooled catalytic immunoglobulins of claim 1, wherein the catalytic reaction entails amide bond cleavage.

8. The pooled catalytic immunoglobulins of claim 1, wherein the catalytic reaction entails peptide bond cleavage.

9. The pooled catalytic immunoglobulins of claim 1, wherein the immunoglobulin class and subclass are selected based on a comparison of catalytic activity of various immunoglobulin classes and subclasses against a specific target antigen.

10. The pooled catalytic immunoglobulins from claim 1, wherein the catalytic reaction entails cleavage of a peptide bond HIV gp120, HIV Tat, Staphylococcal Protein A, CD4 or in amyloid beta peptide.

11. The pooled catalytic immunoglobulins of claim 1, wherein the immunoglobulin class is selected based on a comparison of catalytic cleavage of amide bonds in peptide-aminomethyl coumarin antigens.

12. The pooled catalytic immunoglobulins of claim 1 as a formulation selected for prevention or therapy of HIV-1 infection by intravenous, intravaginal or intrarectal administration.

13. The pooled catalytic immunoglobulins of claim 1 as a formulation selected for the treatment of bacterial infection, septic shock, autoimmune disease, Alzheimer's disease or a combination thereof by intravenous administration.

14. A method for isolating and purifying pooled catalytic immunoglobulins of claim 1 for therapeutic use comprising the step of:
pooling the source fluids obtained from humans and fractionation of the immunoglobulins into a defined class and subclass fraction, wherein said fraction expresses catalytic activity.

15. The method of claim 14, further comprising the step of:
adding a compound that binds and protects the catalytic site during the fractionation procedure, including but not limited to the substrate.

16. The method of claim 14, further comprising the step of:
comparing the catalytic activity of antibody classes and subclasses against an antigen.

17. The method of claim 14, wherein the fractionation step comprises chromatography using antibodies to human IgA, IgM or IgG; or immunoglobulin binding reagents, Protein G, Protein A, Protein L; or electrophilic compounds capable of binding the nucleophilic site of the immunoglobulins; or mixtures and combinations thereof.

18. The method of claim 14, wherein the fractionation procedure includes ion exchange chromatography, gel filtration, chromatography on lectins, chomatofocusing, electrophoresis or isoelectric focusing.

19. A method for treating a patient comprising:
providing an effective amount of pooled catalytic immunoglobulins of a defined class to a patient in need thereof.

20. The method of claim 19, wherein the patient is in need of treatment for a viral infection, bacterial infection, septic shock, immunodeficiency, autoimmune disease, autoinflammatory disease, Alzheimer's disease, or a combination thereof.

21. The method of claim 19, wherein the pooled immunoglobulin preparation is administered by intravenous infusion, intraperitoneal injection or topical application.


SEQUENCE LISTING (19 sequences)

SEQ ID NO: 1 - protein, 40 aa, artificial sequence
  amyloid beta 1-40 peptide
DAEFRHDSGYEVHHQKKVFFAEDVGSNKGAIIGLMVGGVV

SEQ ID NO: 2 - protein, 12 aa, artificial sequence
  amyloid beta 29-40 peptide
GAIIGLMVGGVV

SEQ ID NO: 3 - protein, 16 aa, artificial sequence
  amyloid beta 1-16 peptide
DAEFRHDSGYEVHHQK

SEQ ID NO: 4 - protein, 4 aa, artificial sequence
  synthetic peptide
AAPF

SEQ ID NO: 5 - protein, 4 aa, artificial sequence
  synthetic peptide
IEGR

SEQ ID NO: 6 - protein, 8 aa, human immunodeficiency virus
  epitope residues 429-436 sequence in HIV gp120 protein
EVGKAMYA

SEQ ID NO: 7 - protein, 8 aa, Mycobacterium avium paratuberculosis K10
  epitope sequence of residues 367-374 in 3-ketosteroid-delta-1-dehydrogenase
EVGKAMYA

SEQ ID NO: 8 - protein, 12 aa, human immunodeficiency virus
  epitope sequence of residues 421-432 in HIV gp120 protein
KQIINMWQEVGK

SEQ ID NO: 9 - protein, 13 aa, Streptococcus gordonii
  epitope sequence of residues 77-89 in probable transcription regulator-like protein
KQFIINMSQNVGK

SEQ ID NO: 10 - protein, 13 aa, artificial sequence
  synthetic peptide
KQIINMWQEVGKA

SEQ ID NO: 11 - DNA, 39 bp, human immunodeficiency virus
  nucleotide sequence of clade B gp120 encoding residues 421-433
ccgtatgtaacgaaaaggatgaaagacggtgtacaaata

SEQ ID NO: 12 - DNA, 39 bp, human endogenous retrovirus
  nucleotide sequence for HERV rv-012650
ttagatctgatgaaaaggatgaaagaaatttttcaaaaa

SEQ ID NO: 13 - protein, 13 aa, artificial sequence
  synthetic peptide
KQINNMWGNVGNA

SEQ ID NO: 14 - protein, 20 aa, human immunodeficiency virus
  V3 region residues 306-325 of HIV clade B
YNKRKRIHIGPGRAFYTTKN

SEQ ID NO: 15 - protein, 18 aa, human immunodeficiency virus
  V3 region residues 306-325 of HIV clade C strain ZA009
TRKSMRIGPQVAFYATNG

SEQ ID NO: 16 - protein, 13 aa, human immunodeficiency virus
  V3 region residues 421-433 of HIV clade B
KQIINMWQEVGKA

SEQ ID NO: 17 - protein, 13 aa, human immunodeficiency virus
  V3 region residues 421-433 of HIV clade C strain ZA009
KQIINMWQEVGRA

SEQ ID NO: 18 - protein, 11 aa, artificial sequence
  electrophilic peptide from HIV gp120 421-433
KQIINMWQEVG

SEQ ID NO: 19 - protein, 28 aa, artificial sequence
  electrophilic control peptide
HSDAVFTDNYTRLRKQMAVKKYLNSILN